(19) United States
(12) Patent Application Publication
(10) Pub. No.: US 2009/0042820 A1
(43) Pub. Date: Feb. 12, 2009

(54) Tubulin Binding Anti Cancer Agents And Prodrugs Thereof

(75) Inventors: Mark Matteucci, Portola Valley, CA (US); Jian-Xin Duan, South San Francisco, CA (US); Xiaohong Cai, Burlingame, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Threshold Pharmaceuticals, Inc., Redwood City, CA (US)

(21) Appl. No.: 11/791,420

(22) PCT Filed: Nov. 17, 2005
(86) PCT No.: PCT/US05/42095

§ 371 Date: Jan. 2, 2008

Related U.S. Application Data
(60) Provisional application No. 60/630,422, filed on Nov. 22, 2004.
(60) Provisional application No. 60/726,928, filed on Oct. 14, 2005.

(51) Int. Cl. A61K 31/7056; A61K 31/416; C07D 231/56; A61P 35/00; C07D 215/233; C07H 19/00

(52) U.S. Cl. 514/43; 548/362.5; 514/406; 536/28.7; 546/153

(57) ABSTRACT

Novel tubulin binding compounds and hypoxia activated prodrugs of novel and known tubulin binding compounds useful for treating cancer and other hyperproliferative diseases are disclosed.

### CROSS-REFERENCES TO RELATED APPLICATIONS

This application claims the benefit of U.S. Patent Application No. 60/630,422 filed 22 Nov. 2004; and U.S. Patent Application No. 60/726,928, filed 14 Oct. 2005, the contents of each of which is incorporated herein by reference.

### BACKGROUND OF THE INVENTION

1. Field of the Invention

The present invention provides compositions and methods for treating cancer and other hyperproliferative disease conditions and generally relates to the fields of chemistry, biology, molecular biology, pharmacology, and medicine. In particular, the present invention provides tubulin binding compounds and their prodrugs for treating cancer and other hyperproliferative disease conditions.

2. Description of Related Art

Tubulin-containing structures such as microtubules are important for diverse cellular functions, including chromosome segregation during cell division, intracellular transport, development and maintenance of cell shape, cell motility, and possibly distribution of molecules on cell membranes (Bacher et al., Pure Appl. Chem., 73(9): 1459-1464, 2001). Precipitation and sequestration of tubulin structure interrupts many important biological functions that depend on tubulin via the microtubular class of subcellular organelles. For example, inhibition of tubulin polymerization or prevention of the disassembly of tubulin polymer causes cell cycle arrest which ultimately leads to cell death. As a result, tubulin is a promising target in cancer therapy.

Drug compounds that interfere with tubulin can be useful anti-cancer agents. Three important binding domains have been identified on tubulin where such drug compounds can bind. These drugs have diverse chemical structure (Angerer et al., Curr. Opin. Drug Discov. Dev., 2000, 3(5): 575-584 incorporated herein by reference) suggesting that they can bind on different regions of tubulin. However, a common outcome of tubulin binding of these drugs is that they cause precipitation and sequestration of tubulin.

Clinically used anti-cancer drugs targeting tubulin are of natural origin, namely, the taxanes (paclitaxel, docetaxel), vinca alkaloids (vincristine, vinblastine, vinorelbine), and podophyllotoxins/colchicine. These agents either inhibit polymerization of tubulin (vinca alkaloids/colchicine) or prevent disassembly of microtubules (taxanes). More recently, the natural products epothilone A and B and their analogs were found to be stabilizers of microtubules and highly cytotoxic.

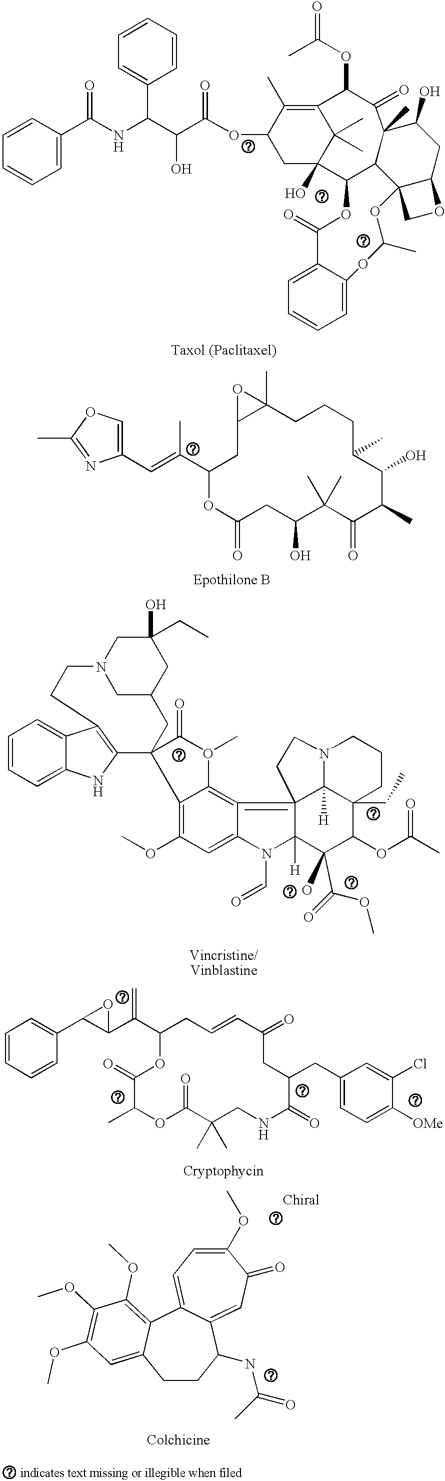

While tubulin-targeting drugs are used clinically to treat cancer, they have several disadvantages (see Bacher et al., supra). The complex chemical structures of these representative drugs make their synthesis difficult and isolating them from natural resources is often difficult. Another major drawback in clinical application of taxanes and vinca alkaloids is the development of neurotoxicity. These drugs interfere with the function of microtubules in axons, which mediate the neuronal vesicle transport. The insolubility of some of these drugs makes administration difficult. Further, over-expression of transmembrane pumps results in development of drug resistance to these agents. These factors limit the potential of these natural products.

Other natural products or derived analogs are known which have increased solubility or potency. However, their complex chemical structure makes their synthesis problematic and limits availability.

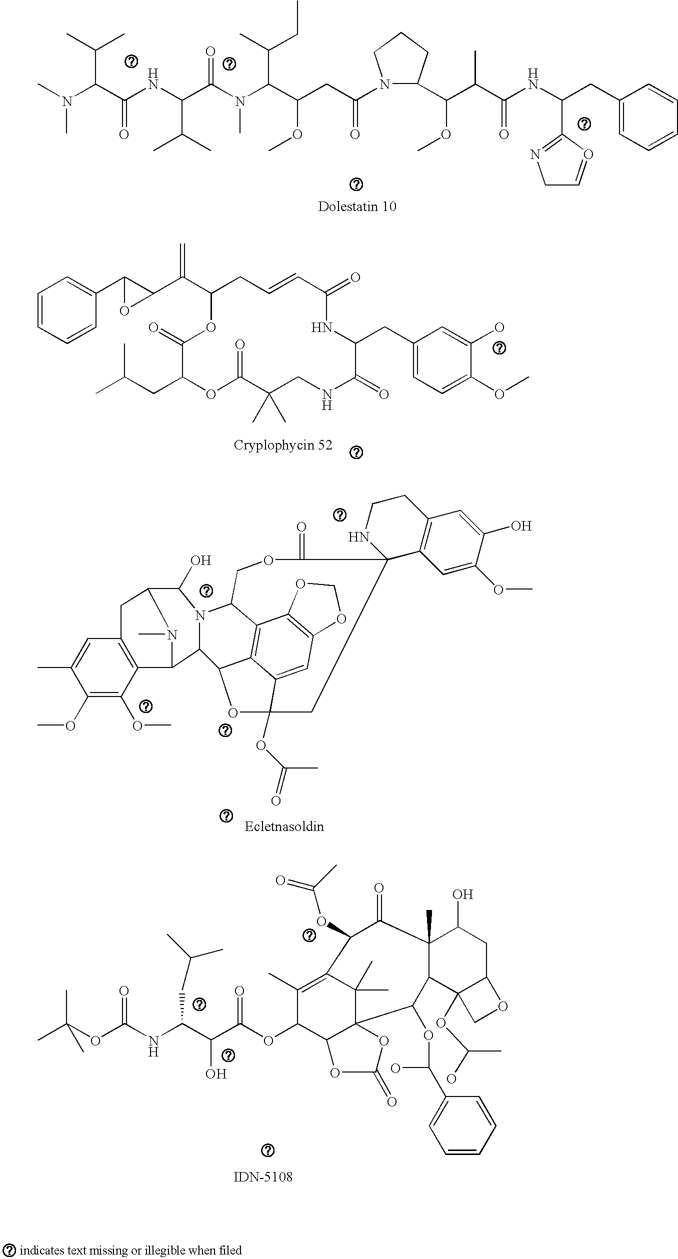

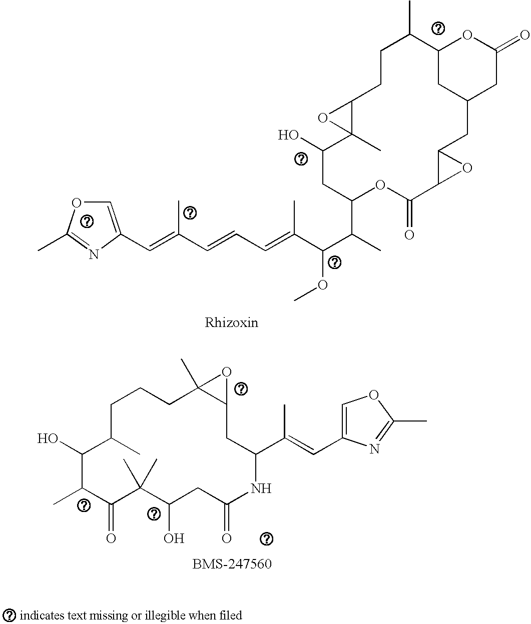

Combretastatin A is a small-molecular weight natural product which binds to the colchicine binding part of tubulin and inhibits tubulin polymerization. Administration of Combretastatin A is problematic because of its low aqueous solubility. A water soluble phosphate prodrug of Combretastatin A is used in therapy. However the phosphate group is hydrolyzed by phosphatases that are not tumor specific, to yield Combretastatin A. Release of insoluble Combretastatin A away from the tumor following such hydrolysis can cause administration problems.

There remains a need for anti-cancer compounds, preferably tubulin binding anti-cancer compounds, especially those that are not substrates of transmembrane pumps and/or do not interfere with the function of axonal microtubules and/or provide an increased therapeutic index in the treatment of cancer. The present invention meets these needs.

### BRIEF SUMMARY OF THE INVENTION

In one aspect, the present invention provides a compound having a formula selected from:

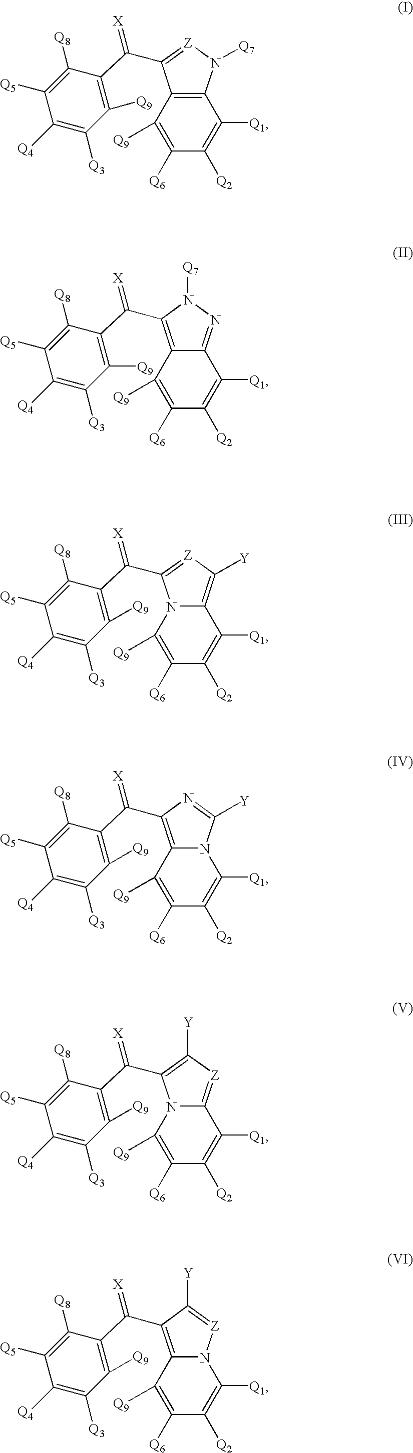

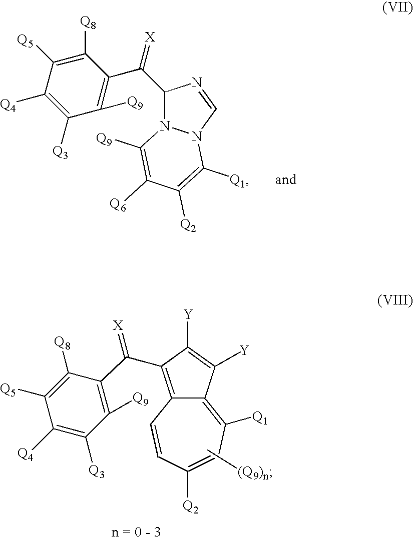

wherein each Q₁, Q₂, and Q₆ independently is hydrogen; halo; amino; C₁-C₆ alkylamino; di C₁-C₆ alkylamino; hydroxyl; C₁-C₆ alkoxy; nitro; cyano; C₁-C₆ alkyl; C₁-C₆ heteroalkyl; C₂-C₆ alkenyl; C₂-C₆ alkynyl; C₃-C₈ cycloalkyl; C₃-C₈ heterocyclyl; aryl; heteroaryl; COR₁₅; SO₂R₁₅; or PO₃R₁₅;

each Q₃-Q₅ is hydrogen; halo; amino; C₁-C₆ alkylamino; di C₁-C₆ alkylamino; hydroxyl; C₁-C₆ alkoxy; nitro; cyano; aryl; heteroaryl; COR₁₅; SO₂R₁₅ or PO₃R₁₅ with the proviso that in any one compound, only one of Q₃-Q₅ is hydrogen; Q₃ and Q₄ together form C₃-C₈ heterocycle, an aryl, or a heteroaryl; or Q₄ and Q₅ together form a C₃-C₈ heterocycle, an aryl, or a heteroaryl;

Q₇ is hydrogen; amino; C₁-C₆ alkylamino; di C₁-C₆ alkylamino; hydroxyl; C₁-C₆ alkoxy; nitro; cyano; C₁-C₆ alkyl; C₁-C₆ heteroalkyl; C₂-C₆ alkenyl; C₂-C₆ alkynyl; C₃-C₈ cycloalkyl; C₃-C₈ heterocyclyl; aryl; heteroaryl; COR₁₈; SO₂R₁₅; PO₃R₁₅ or a monosaccharide; with the proviso that in formula (II) Q₇ excludes hydrogen;

Q₈ is hydrogen; halo; amino; C₁-C₆ alkylamino; di C₁-C₆alkylamino; hydroxyl; C₁-C₆ alkoxy; nitro; cyano; aryl; heteroaryl; COR₁₅; SO₂R₁₅ or PO₃R₁₅;

each Q₉ independently is hydrogen; halo; amino; C₁-C₆ alkylamino; di C₁-C₆ alkylamino; hydroxyl; C₁-C₆ alkoxy; nitro; cyano; aryl; heteroaryl; COR₁₅; SO₂R₁₅ or PO₃R₁₅;

X is O, —NNHR₁₆, NR₁₆, or NOR₁₆;

Y is hydrogen, hydroxyl, or halogen;

Z is —CH— or —N—;

R₁₅ is hydrogen, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl;

R₁₆ is hydrogen, C₁-C₆ alkyl, aryl, C₁-C₆ alkylsulphonyl, arylsulfonyl, C₁-C₆ alkoxycarbonyl, aminocarbonyl, C₁-C₆ alkylaminocarbonyl, di C₁-C₆ alkylaminocarbonyl, C₁-C₆ acyl, aroyl, aminothiocarbonyl, C₁-C₆ alkylaminothiocarbonyl, di C₁-C₆ alkylaminothiocarbonyl, C₁-C₆ thioacyl, or thioaroyl; with the proviso that when X is NR₁₆, R₁₆ excludes hydrogen;

R₁₈ is hydrogen, hydroxyl, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl; or

a tautomer or an individual isomer or a racemic or non-racemic mixture of isomers, a polymorph, a hydrate, a prodrug or a pharmaceutically acceptable salt or solvate thereof. In one embodiment, the compounds are tubulin binding compounds.

In another aspect, the present invention provides a compound of formula (XXI)-(XXVII):

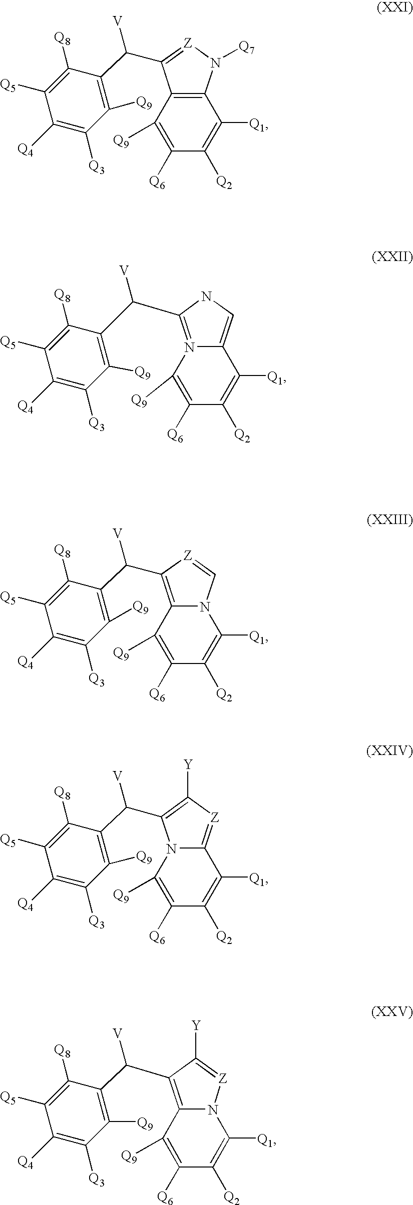

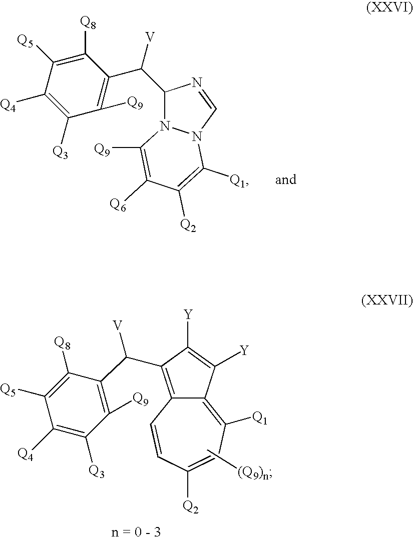

wherein each Q₁, Q₂, and Q₆ independently is hydrogen; halo; amino; C₁-C₆ alkylamino; di C₁-C₆ alkylamino; hydroxyl; C₁-C₆ alkoxy; nitro; cyano; C₁-C₆ alkyl; C₁-C₆ heteroalkyl; C₁-C₆ alkenyl; C₁-C₆ alkynyl; C₃-C₈ cycloalkyl; C₃-C₈ heterocyclyl; aryl; heteroaryl; COR₁₈; SO₂R₁₈; or PO₃R₁₅;

each Q₃-Q₅ is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈; or PO₃R₁₈ with the proviso that in any one compound, only one of Q₃-Q₅ is hydrogen;

Q₇ is hydrogen; halo; amino; C₁-C₆ alkylamino; di C₁-C₆ alkylamino; hydroxyl; C₁-C₆ alkoxy; nitro; cyano; C₁-C₆ alkyl; C₁-C₆ heteroalkyl; C₁-C₆ alkenyl; C₁-C₆ alkynyl; C₃-C₈ cycloalkyl; C₃-C₈ heterocyclyl; aryl; heteroaryl; COR₁₅; SO₂R₁₈; or PO₃R₁₈ or a monosaccharide; with the proviso that in formula (II) Q₇ excludes hydrogen;

Q₈ is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈ or PO₃R₁₈;

each Q₉ independently is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈ or PO₃R₁₈;

V is —NHR₁₆; —NHNHR₁₆; —NHN(R₁₆)₂; —NR₁₆NHR₁₆; or —OR₁₇;

Y is hydrogen, hydroxyl or halogen;

Z is —CH— or —N—;

R₁₅ is hydrogen, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₁-C₆ alkenyl, C₁-C₆ alkynyl, C₁-C₆ cycloalkyl, C₁-C₆ heterocyclyl, aryl, or heteroaryl;

R₁₆ is hydrogen, C₁-C₆alkyl, aryl, C₁-C₆alkylsulphonyl, arylsulfonyl, C₁-C₆ alkoxycarbonyl, aminocarbonyl, C₁-C₆alkylaminocarbonyl, di C₁-C₆ alkylaminocarbonyl, C₁-C₆ acyl, aroyl, aminothiocarbonyl, C₁-C₆ alkylaminothiocarbonyl, di C₁-C₆ alkylaminothiocarbonyl, C₁-C₆ thioacyl, or thioaroyl; and R′ is C₁-C₆ alkyl or aryl; with the proviso that when V is NR₁₆, R₁₆ excludes hydrogen;

R₁₇ is C₁-C₆ alkyl; aryl; or di C₁-C₆ alkylamino;

R₁₈ is hydrogen, hydroxyl, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl; or

a tautomer or an individual isomer or a racemic or non-racemic mixture of isomers, a polymorph, a hydrate, a prodrug or a pharmaceutically acceptable salt or solvate thereof. In one embodiment, these compounds are tubulin binding compounds.

In another aspect, the present invention provides prodrug compounds wherein the novel compound of the invention is bonded to a hypoxic activator (-Hyp) through a hydroxyloxygen (-OHyp) or an amine nitrogen (-NHyp) in the tubulin binding compound.

In another aspect, the present invention provides prodrug compounds of known tubulin binding anti-cancer compounds wherein the tubulin binding compound is bonded to the hypoxic activator (Hyp) through an hydroxyl oxygen (-OHyp) or an amine nitrogen (-NHyp) in the tubulin binding compound.

The hypoxic activator can be nitrobenzene moieties, nitrobenzoic acid amide moieties, nitroazole moieties, nitroimidazole moieties, nitrothiophene moieties, nitrothiazole moieties, nitrooxazole moieties, nitrofuran moieties, and nitropyrrole moieties.

In one embodiment, Hyp is selected from:

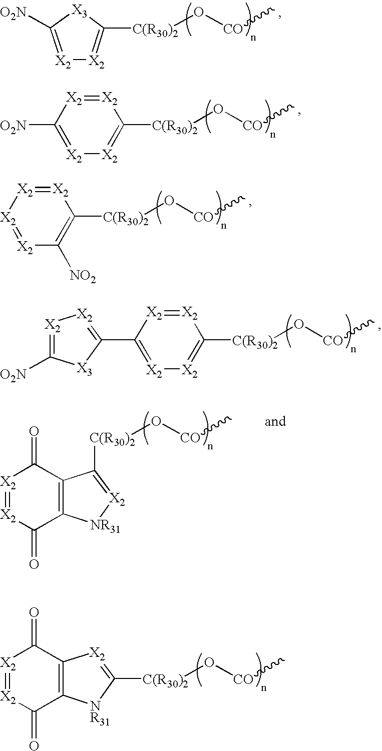

wherein each X₂ is N or CR₃₂;

X₃ is NR₃₁, S, or O;

each R₃₀ is independently hydrogen or alkyl;

R₃₁ is hydrogen, hydroxyl, C₁-C₆ alkyl or heteroalkyl, C₃-C₈ cycloalkyl, heterocyclyl, C₁-C₆ alkoxy, C₁-C₆ alkylamino, C₁-C₆ dialkylamino, aryl or heteroaryl, C₁-C₆ acyl or heteroacyl, aroyl, or heteroaroyl;

R₃₂ is hydrogen, halogen, nitro, cyano, CO₂H, C₁-C₆alkyl or heteroalkyl, C₁-C₆ cycloalkyl, C₁-C₆ alkoxy, C₁-C₆alkylamino, C₁-C₆ dialkylamino, aryl, CON(R₇)₂, C₁-C₆ acyl or heteroacyl, or aroyl or heteroaroyl; and

n=0, 1.

In an additional embodiment, Hyp is selected from

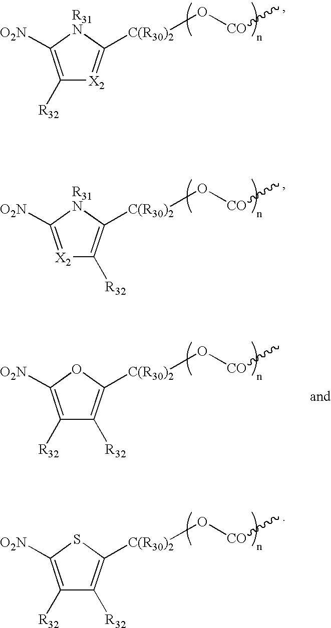

wherein X₂, R₃₀, R₃₁, R₃₂ and n are as defined above.

In one embodiment, the hypoxic activator is a substituted or unsubstituted nitroimidazole moiety. In another embodiment, Hyp is

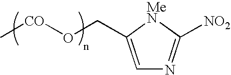

wherein n=0 or 1, provided that in -OHyp n=0.

In another aspect, the present invention provides a pharmaceutical composition comprising a pharmaceutically acceptable carrier and a novel compound or a novel prodrug compound of the invention.

In another aspect, the present invention provides a method of treating cancer comprising administering a therapeutically effective amount of a novel compound or a novel prodrug compound of the invention alone or in combination with one or more other anti-cancer agents to a subject in need of such treatment.

These and other aspects and embodiments of the present invention are described in greater detail in the following section.

### BRIEF DESCRIPTION OF THE SEVERAL VIEWS OF THE DRAWINGS

FIG. 1 illustrates graphically the time course of mice bodyweight recorded during the experiment in the three groups.

FIG. 2 illustrates graphically the time course tumor volume recorded during the experiment in the three groups.

### DETAILED DESCRIPTION OF THE INVENTION

### Definitions

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed to represent a substantial difference over the definition of the term as generally understood in the art.

Before describing the present invention in detail, it is to be understood that this invention is not limited to particular compositions, formulations or process parameters as such may, of course, vary. It is also to be understood that the terminology and examples used herein are for the purpose of describing particular embodiments of the invention only, and are not intended to be limiting.

All patents, patent applications, and publications mentioned herein, whether supra or infra, are hereby incorporated by reference in their entirety.

As used herein, the terms “a” or “an” means “at least one” or “one or more.”

As used herein, “C₁-C₆ alkyl” or (C₁-C₆) alkyl refers to substituted or unsusbstituted straight or branched chain alkyl groups having 1-6 carbon atoms such as, for example, methyl, ethyl, propyl, isopropyl, n-butyl, sec-butyl, tert-butyl, pentyl, 2-pentyl, isopentyl, neopentyl, hexyl, 2-hexyl, 3-hexyl and 3-methylpentyl. A C₁-C₆ alkyl substituent may be covalently bonded to an atom within a molecule of interest via any chemically suitable portion of the C₁-C₆ alkyl group. “C₁-C₆ alkyl” or (C₁-C₆) alkyl may be further substituted with substituents, including for example, hydroxy, amino, mono or di(C₁-C₆)alkyl amino, halogen, C₂-C₆ alkyl ether, cyano, nitro, ethenyl, ethynyl, C₁-C₆ alkoxy, C₁-C₆ alkylthio,

—COOH, —CONH₂, mono- or di-(C₁-C₆)alkyl-carboxamido, —SO₂NH₂, —OSO₂—(C₁-C₆)alkyl, mono or di(C₁-C₆)alkylsulfon-amido, aryl, and heteroary. Substituted C₁-C₆ alkyl groups include, for example, —CH₂—CH₂—OH, —CH₂—CH₂-halogen,
—CH₂—CH₂—NH₂, —CH₂—CH₂—O—CH₂—CH₂—OH, —CH₂—CH₂—CH₂—NH—CH₂—CH₂—OH and —CH₂—CH₂—NH—CH₂—CH₂—OH and the like.

As used herein, the term “Cycloalkyl” refers to a monovalent cyclic hydrocarbon radical of three to seven ring carbons. The cycloalkyl group may have double bonds which may but not necessarily be referred to as “cycloalkene” or “cycloalkenyl”. The cycloalkyl ring may be optionally substituted independently with one, two, or three substituents selected from alkyl, aryl, arylalkyl, heteroaryl, heteroarylalkyl, cycloalkylalkyl, halo, nitro, cyano, hydroxy, alkoxy, amino, mono-alkylamino, di-alkylamino, haloalkyl, haloalkoxy, —COR (where R is hydrogen, alkyl, cycloalkyl, cycloalkylalkyl, phenyl or phenylalkyl),

—(CR′R″)N—COOR (n is an integer from 0 to 5, R′ and R″ are independently hydrogen or alkyl, and R is hydrogen, alkyl, cycloalkyl, cycloalkylalkyl, phenyl or phenylalkyl), or —(CR′R″)ₙ—CONR^{x}R^{y} (where n is an integer from 0 to 5, R′ and R″ are independently hydrogen or alkyl, R^{x} and R^{y} are, independently of each other, hydrogen, alkyl, cycloalkyl, cycloalkylalkyl, phenyl or phenylalkyl). More specifically, the term cycloalkyl includes, for example, cyclopropyl, cyclohexyl, cyclohexenyl, phenylcyclohexyl, 4-carboxycyclohexyl, 2-carboxamido-cyclohexenyl, 2-dimethylaminocarbonyl-cyclohexyl, and the like.

As used herein, the term “Heteroalkyl” means an alkyl radical as defined herein with one, two or three substituents independently selected from cyano,

—OR^{w}, —NR^{x}R^{y}, and —S(O)ₚR^{z} (where p is an integer from 0 to 2), with the understanding that the point of attachment of the heteroalkyl radical is through a carbon atom of the heteroalkyl radical. R^{w} is hydrogen, alkyl, cycloalkyl, cycloalkyl-alkyl, aryl, araalkyl, alkoxycarbonyl, aryloxycarbonyl, carboxamido, or mono- or di-alkylcarbamoyl. R^{x} is hydrogen, alkyl, cycloalkyl, cycloalkyl-alkyl, aryl or araalkyl. R^{y} is hydrogen, alkyl, cycloalkyl, cycloalkyl-alkyl, aryl, araalkyl, alkoxycarbonyl, aryloxycarbonyl, carboxamido, mono- or di-alkylcarbamoyl or alkylsulfonyl. R^{z} is hydrogen (provided that p is 0), alkyl, cycloalkyl, cycloalkyl-alkyl, aryl, araalkyl, amino, mono-alkylamino, di-alkylamino, or hydroxyalkyl. Representative examples include, for example, 2-hydroxyethyl, 2,3-dihydroxy-propyl, 2-methoxyethyl, benzyloxymethyl, 2-cyanoethyl, and 2-methylsulfonyl-ethyl. For each of the above, R^{w}, R^{x}, R^{y}, and R^{z} can be further substituted by amino, fluorine, alkylamino, di-alkylamino, OH or alkoxy. Additionally, the prefix indicating the number of carbon atoms (e.g., C₁-C₁₀) refers to the total number of carbon atoms in the portion of the heteroalkyl group exclusive of the cyano,
—OR^{w}, —NR^{x}R^{y}, or —S(O)ₚR^{z} portions. The term “heteroalkyl,” by itself or in combination with another term, also refers to a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃,
—CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′- and —R′C(O)₂—.

As used herein, the terms “heterocycle”, “heterocyclyl”, “heterocycloalkyl” or “cycloheteroalkyl” means a saturated or unsaturated non-aromatic cyclic radical of 3 to 8 ring atoms in which one to four ring atoms are heteroatoms selected from O, NR (where R is independently hydrogen or alkyl) or S(O)ₚ (where p is an integer from 0 to 2), the remaining ring atoms being C, where one or two C atoms may optionally be replaced by a carbonyl group. The heterocyclyl ring may be optionally substituted independently with one, two, or three substituents selected from alkyl, aryl, arylalkyl, heteroaryl, heteroarylalkyl, cycloalkyl, cycloalkylalkyl, halo, nitro, cyano, hydroxy, alkoxy, amino, mono-alkylamino, di-alkylamino, haloalkyl, haloalkoxy, —COR (where R is hydrogen, alkyl, cycloalkyl, cycloalkylalkyl, phenyl or phenylalkyl), —(CR′R″)ₙ—COOR (n is an integer from 0 to 5, R′ and R″ are independently hydrogen or alkyl, and R is hydrogen, alkyl, cycloalkyl, cycloalkylalkyl, phenyl or phenylalkyl), or —(CR′R″)ₙ—CONR^{x}R^{y} (where n is an integer from 0 to 5, R′ and R″ are independently hydrogen or alkyl, R^{x} and R^{y} are, independently of each other, hydrogen, alkyl, cycloalkyl, cycloalkylalkyl, phenyl or phenylalkyl). More specifically the term heterocyclyl includes, but is not limited to, pyridyl, tetrahydropyranyl, N-methylpiperidin-3-yl, N-methylpyrrolidin-3-yl, 2-pyrrolidon-1-yl, furyl, quinolyl, thienyl, benzothienyl, pyrrolidinyl, piperidinyl, morpholinyl, pyrrolidinyl, tetrahydrofuranyl, tetrahydrothiofuranyl, 1,1-dioxo-hexahydro-1Δ⁶-thiopyran-4-yl, tetrahydroimidazo[4,5-c]pyridinyl, imidazolinyl, piperazinyl, and piperidin-2-onyl. and the derivatives thereof. The prefix indicating the number of carbon atoms (e.g., C₃-C₁₀) refers to the total number of carbon atoms in the portion of the cycloheteroalkyl or heterocyclyl group exclusive of the number of heteroatoms. In one embodiment, R^{x} and R^{y} together is heterocyclyl. More specifically the term aryl includes, but is not limited to, phenyl, biphenyl, 1-naphthyl, and 2-naphthyl, and the substituted forms thereof.

As used herein, “C₁-C₆ alkoxy,” means a substituted or unsubstituted alkyl group of 1 to 6 carbon atoms covalently bonded to an oxygen atom. A C₁-C₆ alkoxy group has the general structure —O—(C₁-C₆ alkyl) wherein alkyl is as described above. C₁-C₆ alkoxy groups include, for example, methoxy, ethoxy, propoxy, isopropoxy, n-butoxy, sec-butoxy, tert-butoxy, n-pentoxy, 2-pentoxy, 3-pentoxy, isopentoxy, neopentoxy, hexoxy, 2-hexoxy, 3-hexoxy, and 3-methylpentoxy.

As used herein, “C₁-C₆ alkoxycarbonyl” refers to an alkoxy group covalently bonded to a carbonyl. A C₁-C₆ alkoxycarbonyl group has the general structure —C(═O)—O—(C₁-C₆)alkyl wherein alkyl is as described above.

As used herein, “C₁-C₆ alkylamino,” means a substituted or unsubstituted alkyl group of 1 to 6 carbon atoms covalently bonded to —NH—. A C₁-C₆ alkylamino group has the general structure —NH—(C₁-C₆)alkyl wherein alkyl is as described above. C₁-C₆ alkylamino groups include, for example, methylamino, ethylamino, propylamino and butylamino.

As used herein, “C₂-C₆ alkyl ether” refers to an ether substituent with 2 to 6 carbon atoms, positioned such that at least one carbon atom is located on either side of the oxygen atom.

As used herein, “aryl” refers to substituted or unsusbstituted moieties that include one or more monocyclic or fused ring aromatic systems. Such moieties include any moiety that has one or more monocyclic or bicyclic fused ring aromatic systems, including but not limited to phenyl and naphthyl.

As used herein, the term “halogen” or “halo” refers to fluorine, chlorine, bromine, and/or iodine.

As used herein, “heteroaryl” refers to substituted or unsusbstituted monocyclic aromatic groups having 5 or 6 ring atoms, or fused ring bicyclic aromatic groups having 8 to 20 atoms, in which the ring atoms are C, O, S, SO, SO₂, or N and at least one of the ring atoms is a heteroatom, i.e., O, S, SO, SO₂, or N. Heteroaryl groups include for example acridinyl, azocinyl, benzimidazolyl, benzofuranyl, benzothio-furanyl, benzothiophenyl, benzoxazolyl, benzothiazolyl, benzotriazolyl, benzotetrazolyl, benzisoxazolyl, benzisothiazolyl, benzimidazolinyl, carbazolyl, NH-carbazolyl, carbolinyl, chromanyl, chromenyl, cinnolinyl, dithiazinyl, furanyl, furazanyl, imidazolidinyl, imidazolinyl, imidazolyl, indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, naphthyridinyl, octahydroisoquinolinyl, oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, pteridinyl, purinyl, pyranyl, pyrazinyl, pyrazolidinyl, pyrazolinyl, pyrazolyl, pyridazinyl, pyridooxazolyl, pyridoimidazolyl, pyridothiazole, pyridinyl, pyridyl, pyrimidinyl, pyrrolyl, quinazolinyl, quinolinyl, quinoxalinyl, quinuclidinyl, tetrahydroisoquinolinyl, tetrahydroquinolinyl, tetrazolyl, thiadiazinyl, thiadiazolyl, thianthrenyl, thiazolyl, thienyl, thienothiazolyl, thienooxazolyl, thienoimidazolyl, thiophenyl, triazinyl and xanthenyl. Unless indicated otherwise, the arrangement of the hetero atoms within the ring may be any arrangement allowed by the bonding characteristics of the constituent ring atoms. Aryl or heteroaryl groups may be further substituted with substituents, including for example, hydroxy, amino, mono or di(C₁-C₆)alkyl amino, halogen, C₂-C₆ alkyl ether, cyano, nitro, ethenyl, ethynyl, C₁-C₆ alkoxy, C₁-C₆ alkylthio, —COOH, —CONH₂, mono- or di-(C₁-C₆)alkyl-carboxamido, —SO₂NH₂, —OSO₂—(C₁-C₆)alkyl, mono or di(C₁-C₆)alkylsulfon-amido, aryl, and heteroaryl.

As used herein, the term “hydroxy(C₁-C₆)alkyl” refers to a substituted or unsubstituted aliphatic group having from 1 to 6 carbon atoms, and further comprising at least one hydroxyl group on the main carbon chain and/or on a side chain. Hydroxy(C₁-C₆)alkyl groups include, for example, —CH₂—CH₂—OH and

### —CH₂—CH₂—CH₂—OH.

As used herein a “protected form of formyl” refers to acetals, oximes, and hydrazones.

As used herein, a “hypoxic activator” or “hypoxia activated trigger” refers to a group or moiety that is capable of releasing another compound, such as an antineoplastic agent or analogs thereof upon hypoxic reduction. In one embodiment, the hypoxic activator is a group that is capable of releasing the antineoplastic agent or analogs thereof upon reduction of the hypoxic activator under hypoxic conditions but does not release any antineoplastic agent or analog under normoxic conditions. For example, and as described in more detail below, one hypoxic activator is a nitroimidazole that may be substituted with a variety of groups. Other examples of hypoxic activators include, but are not limited to, groups based on nitrobenzenes, nitrobenzoic acid amides, nitroazoles, nitroimidazoles, nitrothiophenes, nitrothiazoles, nitrooxazoles, nitrofurans, and nitropyrroles, where each of these classes of moieties may be substituted or unsubstituted, such that the redox potential for the group lies within a range where the group can undergo reduction in the hypoxic regions of a tumor. One of skill in the art will understand, in view of the description herein, how to substitute these and other hypoxia labile protecting groups to provide a redox potential that lies within said range. Additional examples of hypoxic activators are described in Matteucci et al., PCT Publication No. WO 04/087075 and U.S. Pat. Appl. No. 60/695,755 each of which is incorporated herein by reference.

Generally, one of skill in the art can “tune” the redox potential of a hypoxic activator by substituting that activator with electron withdrawing groups, electron donating groups, or some combination of such groups. For example, nitrothiophene, nitrofuranfuran, and nitrothiazole groups may be substituted with one or more electron donating groups, including but not limited to methyl, methoxy, or amine groups, to provide a hypoxic activator with the desired redox potential. In another example, the nitropyrrole moiety can be substituted with an electron withdrawing group, including but not limited to cyano, carboxamide,

—CF₃, and sulfonamide groups, to achieve a group with the desired redox potential. For this purpose, strong electron withdrawing groups such as cyano, sulfone, sulfonamide, carboxamide, or —CF₃, and milder electron withdrawing groups such as —CH₂-halogen, where halogen is —F, —Cl, or —Br, can be used.

As used herein, a “prodrug” is a compound that, after administration, is metabolized or otherwise converted to an active or a more active compound compared to the corresponding prodrug. To produce a prodrug, a cytotoxic, pharmaceutically active compound or precursor thereof can be modified chemically to render it less active or inactive, but the chemical modification is such that an active form of the compound is generated from the resulting prodrug by metabolic or other biological processes. Those of skill in the art recognize, however, that prodrug synthesis does not necessarily require use of the active drug as synthetic intermediate. A prodrug can have, relative to the drug, altered metabolic stability or transport characteristics, fewer side effects, or lower toxicity (for example, see Nogrady, 1985, Medicinal Chemistry A Biochemical Approach, Oxford University Press, New York, pages 388-392).

As used herein, “substituent” refers to a molecular moiety that is covalently bonded to an atom within a molecule of interest.

As used herein, the term “substitution” refers to replacing a hydrogen atom in a molecular structure with a substituent such that the valence on the designated atom (for example 4 for carbon) is not exceeded, and a chemically stable compound (a compound that can be isolated, characterized, and/or tested for biological activity) results.

A combination of substituents or variables is permissible only if such a combination results in a stable or chemically feasible compound. A stable compound or chemically feasible compound is one in which the chemical structure is not substantially altered when kept at a temperature of 4° C. or less, in the absence of moisture or other chemically reactive conditions, for at least a week.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see discussion in Chapter 4 of ADVANCED ORGANIC CHEMISTRY, 4th edition J. March, John Wiley and Sons, New York, 1992).

“Pharmaceutically acceptable salt” of a compound means a salt that is pharmaceutically acceptable and that possesses the desired pharmacological activity of the parent compound. Such salts include:

(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or

(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, trimethylamine, N-methylglucamine, and the like.

“Protecting group” refers to a grouping of atoms that when attached to a reactive group in a molecule masks, reduces or prevents that reactivity. Examples of protecting groups can be found in T. W. Greene and P. G. Wuts, PROTECTIVE GROUPS IN ORGANIC CHEMISTRY, (Wiley, 2nd ed. 1991) and Harrison and Harrison et al., COMPENDIUM OF SYNTHETIC ORGANIC METHODS, Vols. 1-8 (John Wiley and Sons. 1971-1996). Representative amino protecting groups include formyl, acetyl, trifluoroacetyl, benzyl, benzyloxycarbonyl (CBZ), tert-butoxycarbonyl (Boc), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC) and the like. Representative hydroxy protecting groups include those where the hydroxy group is either acylated or alkylated such as benzyl and trityl ethers as well as alkyl ethers, tetrahydropyranyl ethers, trialkylsilyl ethers and allyl ethers.

As used herein, “patient” or “subject” typically refers to a human but more generally refers to a mammal. Those of skill in the art will appreciate that the methods and compositions of the invention can be used to treat cancer or other hyperproliferative diseases in any mammal, including non-human primates, and experimental models of human cancers. In one embodiment, the patient is a human patient.

As used herein, “treating” a condition or patient refers to taking steps to obtain beneficial or desired therapeutic results, including clinical results. Beneficial or desired therapeutic results include, but are not limited to, alleviation or amelioration of one or more symptoms of cancer, diminishment of extent of disease, delay or slowing of disease progression, palliation or stabilization of the disease state, and other beneficial results, as described below.

As used herein, “reduction” of a symptom or symptoms (and grammatical equivalents of this phrase) means decreasing of the severity or frequency of the symptom(s) or eliminating the symptom(s).

As used herein, “administering” or “administration of” a drug to a subject (and grammatical equivalents of this phrase) can include direct administration, including self-administration and/or indirect administration, including the act of prescribing a drug. For example, as used herein, a physician who instructs a patient to self-administer a drug and/or provides a patient with a prescription for a drug is administering the drug to the patient.

As used herein, an “effective amount” or a “therapeutically effective amount” of a drug is an amount of a drug that, when administered to a subject with cancer or any other hyperproliferative disease condition, will have (i) the intended therapeutic effect, e.g., alleviation, amelioration, palliation or elimination of one or more manifestations of cancer or other disease in the subject; or (ii) a prophylactic effect, e.g., preventing or delaying the onset (or reoccurrence) of disease or symptoms or reducing the likelihood of the onset (or reoccurrence) of disease or symptoms. The full therapeutic or prophylactic effect does not necessarily occur by administration of one dose and can occur only after administration of a series of doses. Thus, a therapeutically or prophylactically effective amount can be administered in one or more administrations.

As used herein, a “prophylactically effective amount” of a drug is an amount of a drug that, when administered to a subject, will have the intended prophylactic effect, e.g., preventing or delaying the onset (or reoccurrence) of disease or symptoms, or reducing the likelihood of the onset (or reoccurrence) of disease or symptoms. The full prophylactic effect does not necessarily occur by administration of one dose, and may occur only after administration of a series of doses. Thus, a prophylactically effective amount may be administered in one or more administrations.

As used herein, a “pharmaceutically acceptable carrier or excipient” means a carrier or excipient that is useful in preparing a pharmaceutical composition that is generally safe, non-toxic and neither biologically nor otherwise undesirable, and includes a carrier or excipient that is acceptable for veterinary use as well as human pharmaceutical use. Examples include any and all solvents, dispersion media, coatings, antibacterial and antifungal agents, isotonic agents, absorption delaying agents, and the like, used in the preparation of a pharmaceutical composition. The use of such media and agents for pharmaceutical active substances is well known in the art. Except insofar as any conventional media or agent is incompatible with the active ingredient, its use in the pharmaceutical compositions or pharmaceutical formulations of the invention is contemplated. Supplementary active ingredients can be incorporated into the compositions of the invention. A “pharmaceutically acceptable carrier or excipient” as used in the specification and claims includes both one and more than one such carrier or excipient.

### Compounds

The compounds of the invention can be described in part as compounds which can bind to tubulin, and prodrugs thereof comprising a hypoxic activator. In one embodiment, the compounds are anti-cancer compounds which can bind to tubulin, and prodrugs thereof comprising a hypoxic activator. In one embodiment, the compounds are synthetic, anti-cancer compounds which can bind to tubulin, and prodrugs thereof comprising a hypoxic activator. While a number of synthetic small-molecule tubulin binding anti-cancer compounds are known, none of them have been approved for cancer therapy. A number of synthetic tubulin binding compounds bind to the colchicine binding region of tubulin, and show structural and functional similarities with colchicine.

Colchicine binds to tubulin and interferes with the function of the mitotic spindles causing depolymerization and disappearance of tubulin polymers known as microtubules. Disappearance of microtubules disrupts spindle formation as a result of which Colchicine arrests mitosis in metaphase. Cancer cells with a high rate of cell division are affected by mitotic arrest and high concentrations of Colchicines can completely prevent cells from entering mitosis, resulting in cell death.

In addition, a Colchicine-like tubulin binder such as Combretastatin A can selectively target the vascular system of tumors. The morphological changes induced in the endothelial cells of the tumor's blood vessels irreversibly shut down the blood flow to cancer cells while leaving the blood supply to healthy cells intact.

Comparing the structures of tubulin binding compounds Colchicine and Combretastatin A indicates that the pharmacophore responsible for their tubulin binding contains two aryl rings functionalized with methoxy and/or hydroxyl groups. A number of di- and tri-aryl compounds having tubulin binding ability have been synthesized (see, for example, Nam et al., Curr. Med. Chem., 2003, 10:1697-1722 and Hsieh et al., US Patent Publication No. 2003/0195244, each of which is incorporated herein by reference). Heterocyclic indole, benzofuran, and benzothiophene containing tubulin binding di- and tri-aryl compounds constitute a sub-class of these compounds (see Nam et al. supra). These compounds, for example, have an aromatic moiety such as an aryl or an aroyl

(—CO-Aryl) moiety or a CH group in the 2 position (as illustrated in structures below).

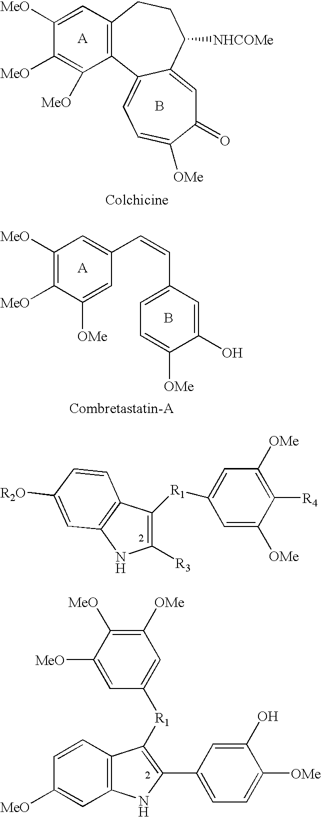

R₁ is CO or CH₂; R₃ is H, methyl, aryl or aroyl; and R₂ and R₄ are methyl or OMe.

The present invention arises in part out of the unexpected discovery that replacing the aromatic moiety or the CH group with a N atom at the 2-position on the heterocyclic portion of these compounds yields an indazole compound with anticancer activity

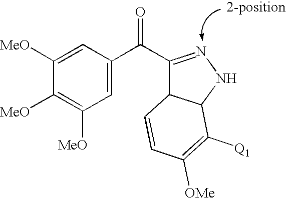

wherein Q₁=H or

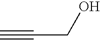

In the above compound when Q₁ was substituted with a

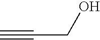

moiety the corresponding propargylic compound demonstrated about 10 fold higher cancer cell killing ability in an antiproleferation assay, compared to a corresponding compound having Q₁ as H.

Compounds of the present invention also arise in part out of the discovery that the proper spatial disposition (i.e. a conformation) between the aryl moieties in the tubulin binding compounds can be stabilized by incorporating a hydrogen bond donor in place of the keto group of the aroyl moiety. As illustrated below, steric repulsion between proximal atoms can move rings A and B from co-planarity. Hydrogen bonding stabilizes the desired conformation of a compound of the present invention in a colchicines-like conformation.

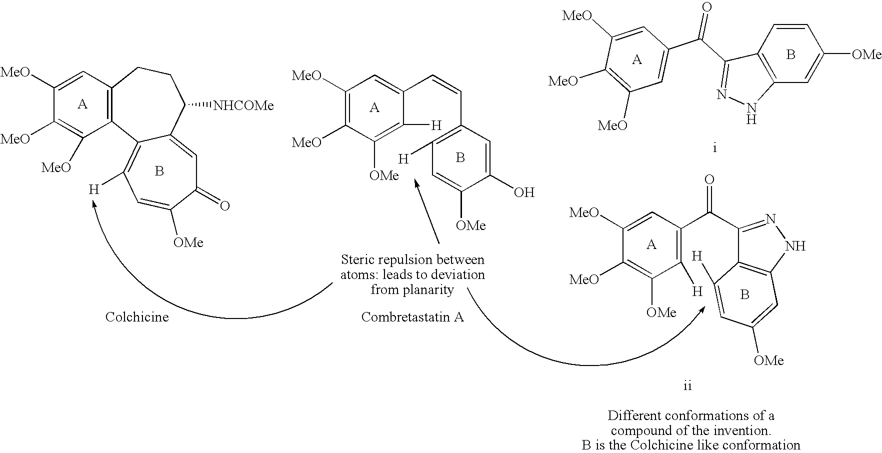

In one embodiment, a compound of the present invention in one conformation can have a colchicine-like structure; while in other conformations have a non-coichicine like structure. These latter conformations can reduce the effective tubulin binding of the compound. By providing a group which is a hydrogen bond donor, the spatial disposition of the aryl groups can be modulated toward a tubulin binding conformation via hydrogen bonding.

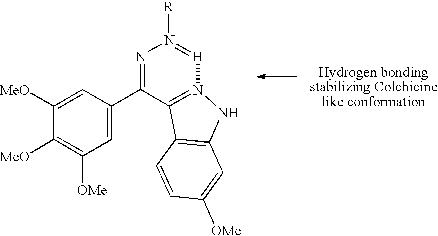

In addition to the nitrogen atom (—N═) at the 2 position, in one embodiment, this invention further provides, an enol ═C(OH)— and a ═C(halogen)-moiety at that position as hydrogen bond acceptors. Also, an —NH— or a ═CH— group at the same position can act as a hydrogen bond donor and stabilize a tubulin binding conformation by hydrogen bonding to a hydrogen bond acceptor suitably disposed in the molecule.

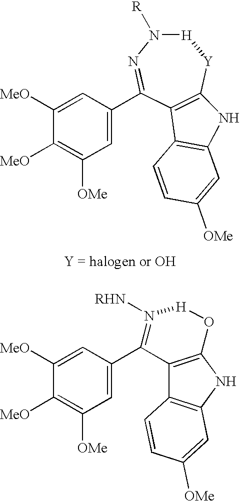

The present invention also provides prodrugs of known and novel tubulin binding compounds of this invention. To understand the prodrug aspect of the invention, an understanding of tumor biology is helpful. Cancer cells generally divide more frequently than normal cells. Tubulin binding-drug mediated cancer therapies include cytotoxic agents selective for dividing cells. For example, tubulin binding compounds target cancer cells, as opposed to normal cells, generally because cancer cells undergo cell division more frequently than normal cells.

However, drugs targeting dividing cells do not kill all of the cancer cells in the solid tumor. One reason for the lack of this complete killing is that cancer cells can acquire mutations that confer drug resistance. Another is that not all cancer cells divide more frequently than normal cells. These slowly-dividing cancer cells are generally located in the hypoxic region of the tumor and can be as, or even more, insensitive to such inhibitors as normal cells. The formation and consequences of the tumor hypoxic region is described below.

As a tumor grows, it requires a blood supply and, consequently, growth of new vasculature. The new vasculature that supports tumor growth is often disordered, leaving significant regions of the tumor under-vascularized and even the vascularized regions subject to intermittent blockage. Cells in these regions are unable to generate the energy required for cell division. These under-vascularized and blocked regions of the tumor become hypoxic—they have a lower oxygen concentration than the corresponding normal tissue. Thus, the median oxygen concentration of only ten percent of solid tumors falls in the normal range of 40-60 mm Hg, and fifty percent of solid tumors exhibit median oxygen concentrations of less than 10 mm Hg.

The hypoxic regions of the tumor can constitute a significant reservoir of cancer cells resistant to therapy. Generally, low tumor oxygen levels are associated with a poor response to therapy, increased metastases, and poor survival. In the hypoxic region of a tumor, cancer cells do not divide significantly faster than normal cells, and can be resistant to therapeutic agents such as tubulin binding compounds that target dividing cells.

However, the hypoxic region is conducive to biochemical reduction that can be used to generate reduced derivatives of a variety of chemical groups (see Workman et al., 1993, Cancer and Metast. Rev. 12: 73-82), and prodrugs of cytotoxins can be developed to exploit such hypoxic regions (see, Matteucci et al., PCT Publication No. WO 04/087075). Compounds of the present invention arise in part out of the discovery that, cancer cells in the hypoxic region can be targeted by prodrug compounds comprising a tubulin binding cytotoxin and a hypoxia labile protecting group. The hypoxic cells of the tumor generate the active toxin from the inactive, relatively non-toxic prodrug. The active drug diffuses from the hypoxic cells and kills the cancer cells in adjacent regions, including the more frequently dividing cells.

The hypoxic region acts as a drug-factory to produce a cytotoxin within a tumor for killing adjacent normoxic cancer cells leading to a higher concentration of the cytotoxin within the tumor, relative to normal tissues. As a result, by employing a prodrug to generate the cytotoxin within the tumor, toxic side-effects arising due to normal cell toxicity can be reduced. After the cancer cells die in the normoxic region of the tumor, a hypoxic region can become normoxic and start dividing. At this point, such cells can be killed by the tubulin binding cytotoxins generated from the prodrug compounds of this invention, or by administering compounds of this invention in combination with other cytoxins, including for example, tubulin binding compounds and other anti-cancer cytotoxins.

A suitable site for incorporating a hypoxic activator on the tubulin binding compounds of the invention to yield a prodrug was discovered as

provided below. For example methylation of

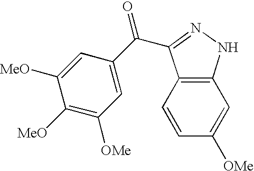

yields two isomeric N-methyl derivatives each of which were less potent in killing cancer cells than the starting toxin. The compound

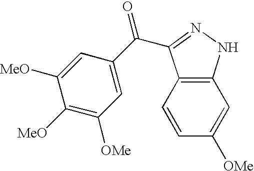

when alkylated with N-1-methyl-2-nitro-5-imidazolemethyl group can yield a hypoxia activated prodrug. Under hypoxic conditions the hypoxic activator is reduced and removed yielding the potent toxin.

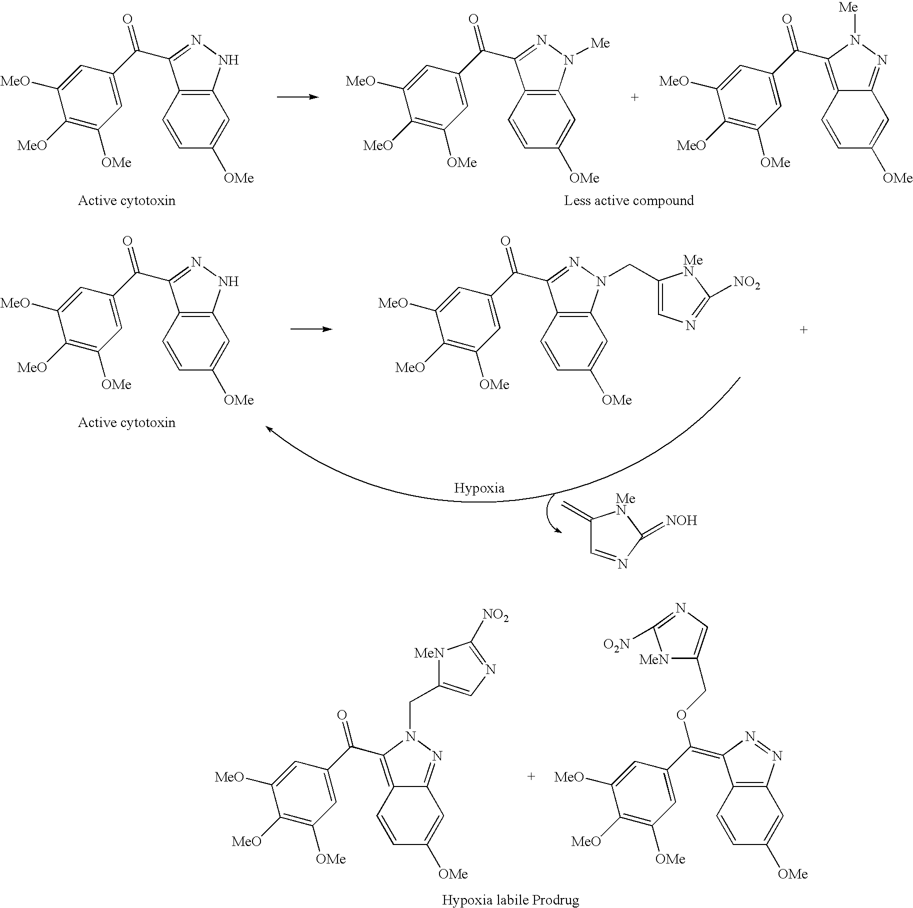

The hypoxic activator can be attached to the nitrogen atoms via a —CO₂-linker as well as shown schematically below.

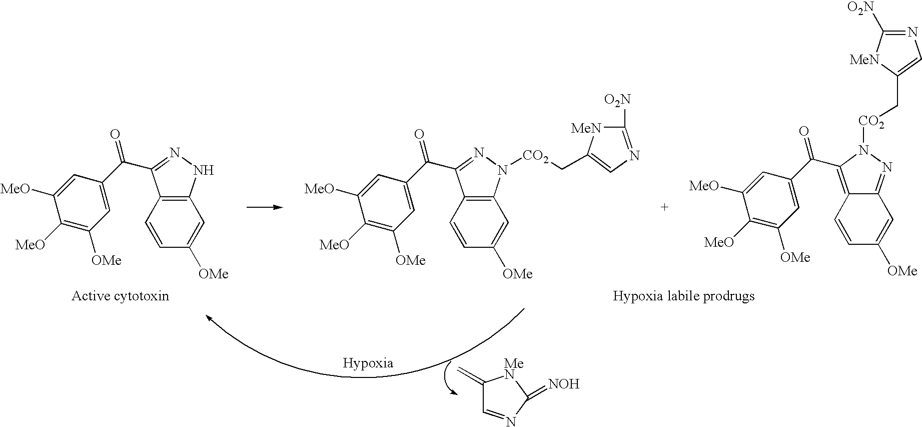

The present invention also provides novel prodrugs of previously known tubulin binding anti-cancer compounds. In this aspect, the tubulin binding compound is bonded to the hypoxic activator (Hyp) through a hydroxyl oxygen (-OHyp) or an amine nitrogen (-NHyp) in the tubulin binding compound to yield a hypoxia active prodrug. The hypoxic activator can be electron deficient nitrobenzene moieties, electron deficient nitrobenzoic acid amide moieties, nitroazole moieties, nitroimidazole moieties, nitrothiophene moieties, nitrothiazole moieties, nitrooxazole moieties, nitrofuran moieties, and nitropyrrole moieties. In one embodiment, the hypoxic activator is a substituted or unsubstituted nitroimidazole moiety.

In one embodiment, the hypoxic activator (Hyp) is selected from:

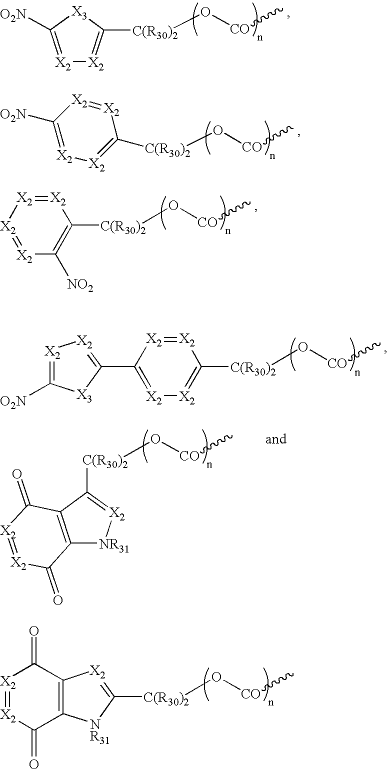

wherein each X₂ is N or CR₃₂;

X₃ is NR₃₁, S, or O;

each R₃₀ is independently hydrogen or alkyl;

R₃₁ is hydrogen, hydroxyl, C₁-C₆ alkyl or heteroalkyl, C₃-C₈ cycloalkyl, heterocyclyl, C₁-C₆alkoxy, C₁-C₆ alkylamino, C₁-C₆ dialkylamino, aryl or heteroaryl, C₁-C₆ acyl or heteroacyl, aroyl, or heteroaroyl;

R₃₂ is hydrogen, halogen, nitro, cyano, CO₂H, C₁-C₆ alkyl or heteroalkyl, C₁-C₆ cycloalkyl, C₁-C₆ alkoxy, C₁-C₆ alkylamino, C₁-C₆ dialkylamino, aryl, CON(R₇)₂, C₁-C₆ acyl or heteroacyl, or aroyl or heteroaroyl; and

n=0, 1.

In an additional embodiment, Hyp is selected from

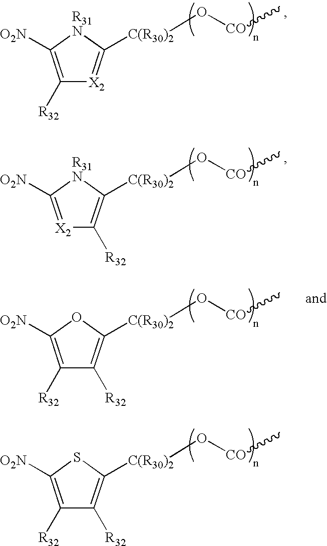

wherein X₂, R₃₀, R₃₁, R₃₂ and n are as defined above.

In another embodiment, Hyp is

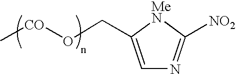

wherein n=0 or 1, provided that in -OHyp n=0. For example, the tubulin binding compounds can be derivatized to yield prodrugs having the following structures

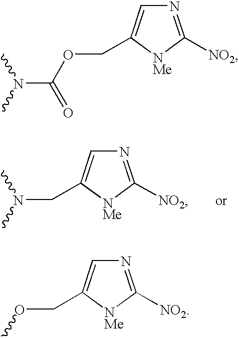

These derivatized compounds in general are less active or inactive compared to the parent compound yielding a hypoxia activated prodrug compound. In certain embodiments, the prodrug compounds demonstrate a 5-1000 fold loss of anticancer activity upon derivatization with respect to the starting compound. In general, such activity data can be obtained from described structure activity relationship data, and via search tools such as SciFinder from the American Chemical Society, Beilstein from MDL Software, US Patent and Trademark Office's Patent and Patent Application search, and European Patent Office's Patent search.

In one aspect, the present invention provides compounds of formulas (I)-(VIII):

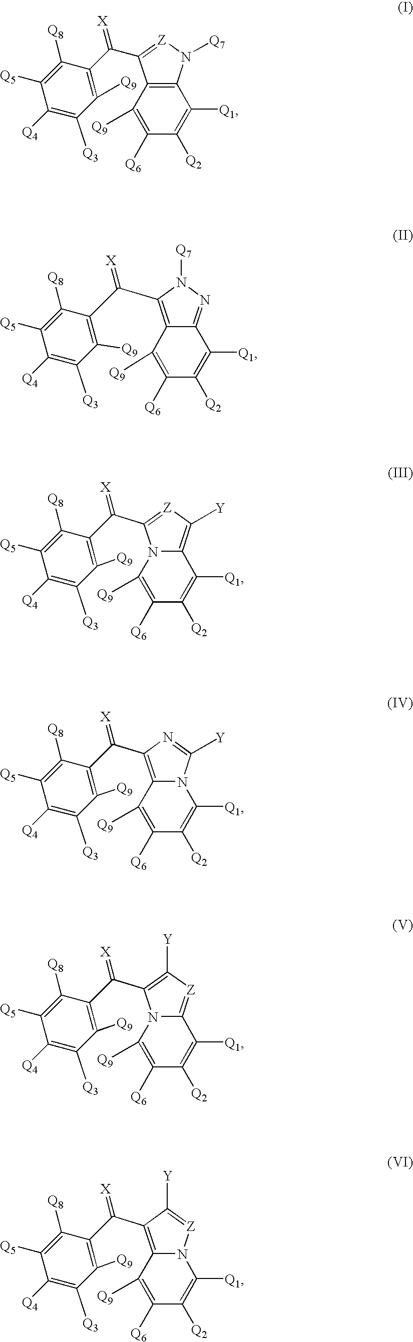

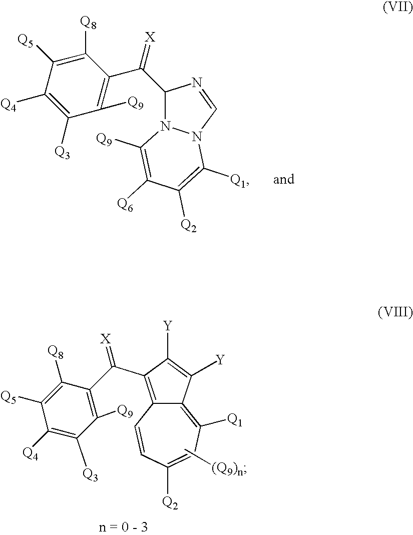

wherein each Q₁, Q₂, and Q₆ independently is hydrogen; halo; amino; C₁-C₆ alkylamino; di C₁-C₆ alkylamino; hydroxyl; C₁-C₆ alkoxy; nitro; cyano; C₁-C₆ alkyl; C₁-C₆ heteroalkyl; C₂-C₆ alkenyl; C₂-C₆ alkynyl; C₃-C₈ cycloalkyl; C₃-C₈ heterocyclyl; aryl; heteroaryl; COR₁₅; SO₂R₁₅; or PO₃R₁₅;

each Q₃-Q₅ is hydrogen; halo; amino; C₁-C₆ alkylamino; di C₁-C₆ alkylamino; hydroxyl; C₁-C₆ alkoxy; nitro; cyano; aryl; heteroaryl; COR₁₅; SO₂R₁₅ or PO₃R₁₅ with the proviso that in any one compound, only one of Q₃-Q₅ is hydrogen; Q₃ and Q₄ together form C₃-C₈ heterocycle, an aryl, or a heteroaryl; or Q₄ and Q₅ together form a C₃-C₈ heterocycle, an aryl, or a heteroaryl;

Q₇ is hydrogen; amino; C₁-C₆ alkylamino; di C₁-C₆ alkylamino; hydroxyl; C₁-C₆ alkoxy; nitro; cyano; C₁-C₆ alkyl; C₁-C₆ heteroalkyl; C₂-C₆ alkenyl; C₂-C₆ alkynyl; C₃-C₈ cycloalkyl; C₃-C₈ heterocyclyl; aryl; heteroaryl; COR₁₅; SO₂R₁₈; PO₃R₁₈ or a monosaccharide; with the proviso that in formula (II) Q₇ excludes hydrogen;

Q₈ is hydrogen; halo; amino; C₁-C₆ alkylamino; di C₁-C₆alkylamino; hydroxyl; C₁-C₆ alkoxy; nitro; cyano; aryl; heteroaryl; COR₁₈; SO₂R₁₈ or PO₃R₁₈;

each Q₉ independently is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈ or PO₃R₁₈;

X is O, —NNHR₁₆, or NR₁₆, or NOR₁₆;

Y is hydrogen, hydroxyl, or halogen;

Z is —CH— or —N—;

R₁₅ is hydrogen, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl;

R₁₆ is hydrogen, C₁-C₆ alkyl, aryl, C₁-C₆ alkylsulphonyl, arylsulfonyl, C₁-C₆ alkoxycarbonyl, aminocarbonyl, C₁-C₆ alkylaminocarbonyl, di C₁-C₆ alkylaminocarbonyl, C₁-C₆ acyl, aroyl, aminothiocarbonyl, C₁-C₆ alkylaminothiocarbonyl, di C₁-C₆ alkylaminothiocarbonyl, C₁-C₆ thioacyl, or thioaroyl; with the proviso that when X is NR₁₆, R₁₆ excludes hydrogen;

R₁₈ is hydrogen, hydroxyl, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl; or

a tautomer or an individual isomer or a racemic or non-racemic mixture of isomers, a polymorph, a hydrate, a prodrug or a pharmaceutically acceptable salt or solvate thereof.

In one embodiment, the present invention provides compounds of formulas (I)-(VIII), wherein Q₁ is hydrogen; halo; cyano; nitro; COR₁₈; SO₂R₁₈; PO₃R₁₈; ≡R₁₃ or

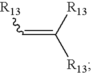

### Q₂ is ≡R₁₃;

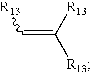

C₁-C₆ alkoxy; halo; amino; or hydroxy; each Q₃, Q₄ and Q₅ independently is hydrogen, C₁-C₆ alkoxy, halo, amino, hydroxyl, Q₃ and Q₄ together is methylenedioxy, or Q₄ and Q₅ together is methylenedioxy, provided that in any compound only one of the Q₃, Q₄ and Q₅ is hydrogen;

Q₇ is C₁-C₆ alkyl optionally substituted independently with one or more aryl, heteroaryl, hydroxyl, amino, C₁-C₆alkylamino, di C₁-C₆alkylamino, CO₂H, or CONH₂; COR₁₈; SO₂R₁₈; or PO₃R₁₈; or a monosaccharide;

each Q₈ and Q₉ is hydrogen;

R₁₃ is hydrogen; C₁-C₆ alkyl, C₁-C₆ heteroalkyl, C₃-C₈ cycloalkyl, C₃-C₈ heterocyclyl, aryl, or heteroalkyl each optionally substituted with hydroxyl, C₁-C₆ alkoxy, amino, C₁-C₆ alkylamino, di C₁-C₆ alkylamino, NHCOR₁₅, or COR₁₈; and

R₁₈ is hydrogen, hydroxyl, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl; or a tautomer or an individual isomer or a racemic or non-racemic mixture of isomers, a polymorph, a hydrate, a prodrug or a pharmaceutically acceptable salt or solvate thereof.

In an additional embodiment, the present invention provides compounds of formulas (I)-(VIII), wherein Q₁ is hydrogen; halo; cyano; CO₂H; CONH₂; ≡R₁₃; or ≡R₁₃; and each Q₂-Q₆ independently is hydrogen, C₁-C₆, alkoxy; halo; amino; or hydroxy; with the proviso that in any compound only one of the Q₃, Q₄, and Q₅ is hydrogen; or a tautomer or an individual isomer or a racemic or non-racemic mixture of isomers, a polymorph, a hydrate, a prodrug or a pharmaceutically acceptable salt or solvate thereof.

In one embodiment, the present invention provides compounds of formulas (I-i), (III-i), (IV-i), (V-i), (VI-i), (VII-i) and (VII-i):

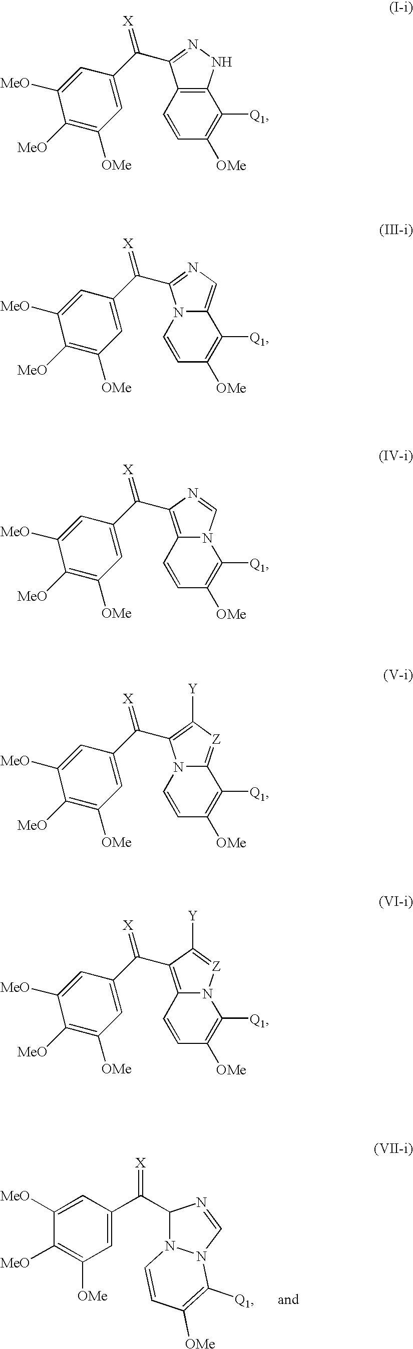

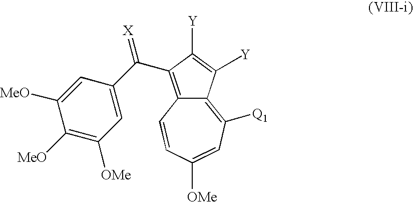

wherein Q₁ is

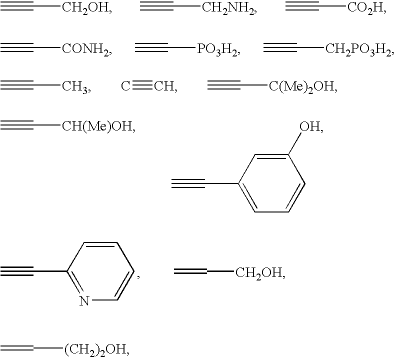

—CH₂—CH₂—OH, —CH₂—CH₂—CH₂—OH, —CONH₂, —CO₂H, —CN, or halo; and X, Y, and Z are defined as above, or a tautomer or an individual isomer or a racemic or non-racemic mixture of isomers, a polymorph, a hydrate, a prodrug or a pharmaceutically acceptable salt or solvate thereof. In another embodiment, the present invention provides compounds of formulas (VIII)-(XIII) wherein X is O.

In another embodiment, the present invention provides the compound of formula:

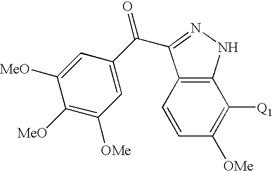

wherein Q₁ is defined as above, or a tautomer or an individual isomer or a racemic or non-racemic mixture of isomers, a polymorph, a hydrate, a prodrug or a pharmaceutically acceptable salt or solvate thereof. In one embodiment, Q₁ is

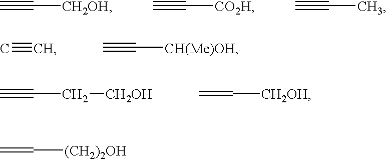

In another embodiment, the present invention provides the compound of formula:

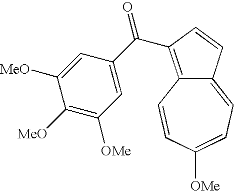

In one embodiment, the present invention provides compounds of formulas (IX)-(XIII):

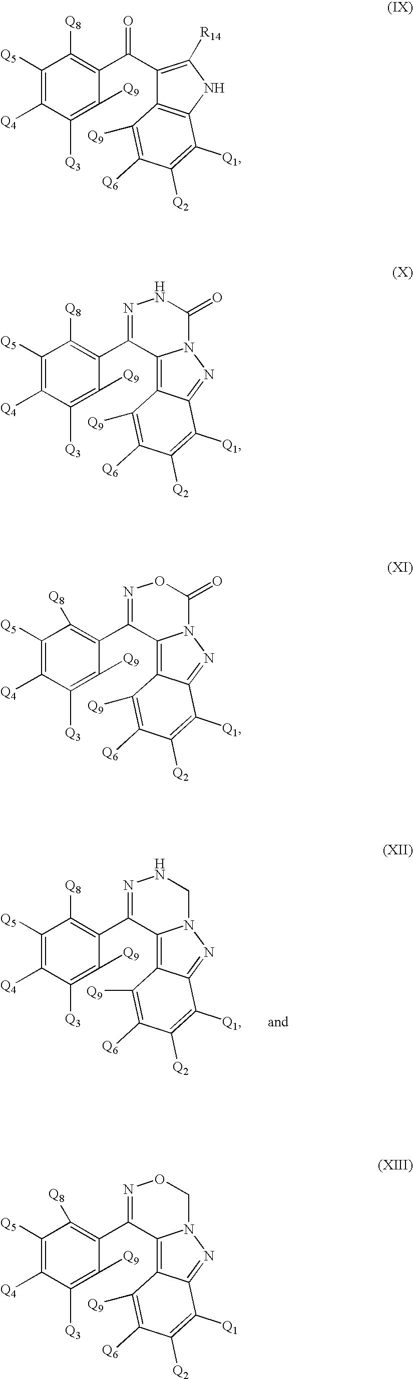

wherein R₁₄ is H, Me, or B(OH)₂; and Q1-Q9 are as defined above; or

a tautomer or an individual isomer or a racemic or non-racemic mixture of isomers, a polymorph, a hydrate, a prodrug or a pharmaceutically acceptable salt or solvate thereof.

In another aspect, the present invention provides compounds of formula (IX-i)-(XIII-i)

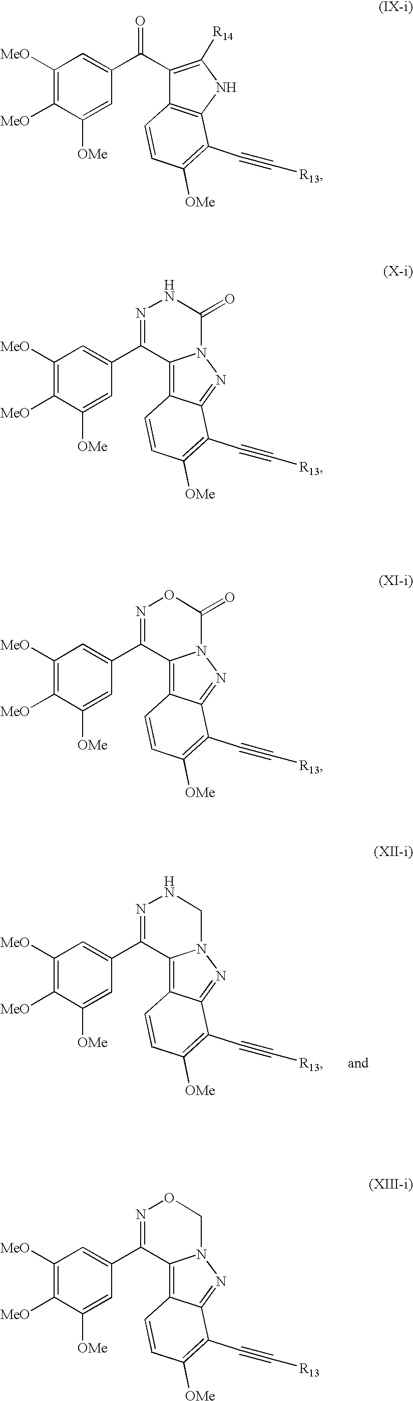

wherein R₁₃ is H, Me, CH₂OH, CH(Me)OH, CH₂CH₂OH, CH₂NH₂, CH₂PO₃H2, PO₃H₂, CO₂H, or CONH₂ and R₁₄ is H, Me, or B(OH)₂; or a tautomer or an individual isomer or a racemic or non-racemic mixture of isomers, a polymorph, a hydrate, a prodrug or a pharmaceutically acceptable salt or solvate thereof.

In another embodiment, the present invention provides a compound of formula (XIV):

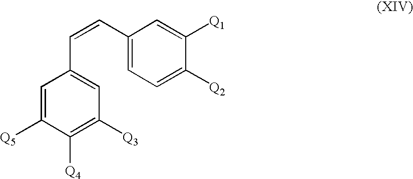

wherein

Q₁ is ≡R₁₃ or

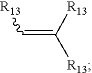

Q₂ is ≡R₁₃;

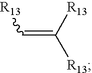

C₁-C₆ alkoxy; halo; amino; or hydroxy;

each Q₃-Q₅ is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈, or PO₃R₁₈; Q₃ and Q₄ together form C₃-C₈ heterocycle, an aryl, or a heteroaryl; or Q₄ and Q₅ together form a C₃-C₈ heterocycle, an aryl, or a heteroaryl; with the proviso that in any one compound, only one of Q₃-Q₅ is hydrogen;

R₁₃ is hydrogen; C₁-C₆ alkyl, C₁-C₆ heteroalkyl, C₃-C₈ cycloalkyl, C₃-C₈ heterocyclyl, aryl, or heteroalkyl each optionally substituted with hydroxyl, C₁-C₆ alkoxy, amino, C₁-C₆ alkylamino, di C₁-C₆ alkylamino; COR₁₈ or NHCOR₁₅;

R₁₅ is hydrogen, hydroxyl, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl; or

a tautomer or an individual isomer or a racemic or non-racemic mixture of isomers, a polymorph, a hydrate, a prodrug or a pharmaceutically acceptable salt or solvate thereof. In one embodiment, the present invention provides a compound of formula (XIV), wherein Q₁ is ≡R₁₃; or

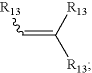

and each Q₂-Q₅ independently is hydrogen, C₁-C₆ alkoxy; halo; amino; or hydroxy; with the proviso that in any compound only one of the Q₃, Q₄ and Q₅ is hydrogen; or a tautomer or an individual isomer or a racemic or non-racemic mixture of isomers, a polymorph, a hydrate, a prodrug or a pharmaceutically acceptable salt or solvate thereof.

In one embodiment, the present invention provides compounds selected from:

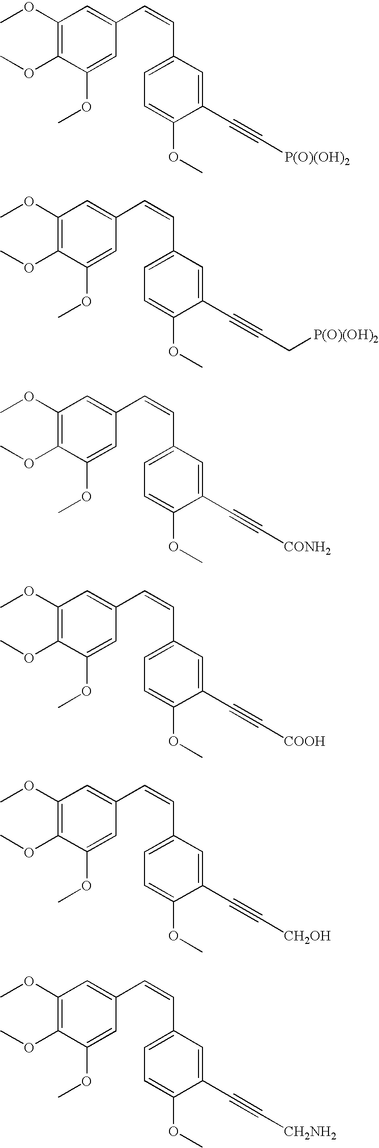

In one embodiment, the present invention provides the compound of formula:

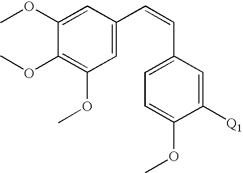

wherein Q₁ is

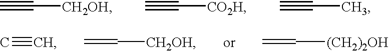

and a tautomer or an individual isomer or a racemic or non-racemic mixture of isomers, a polymorph, a hydrate, a prodrug or a pharmaceutically acceptable salt or solvate thereof.

In another aspect, the present invention provides a compound of formula (XV):

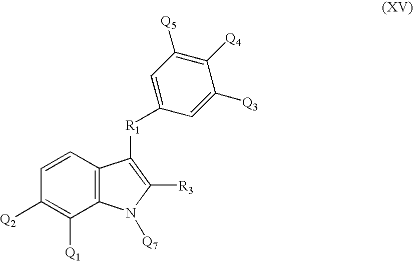

wherein

Q₁ is ≡R₁₃ or

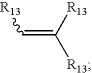

Q₂ is ≡R₁₃.

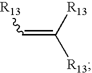

C₁-C₆ alkoxy; halo; amino; or hydroxy;

each Q₃, Q₄, and Q₅ independently is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈; or PO₃R₁₈ with the proviso that in any one compound, only one of Q₃-Q₅ is hydrogen;

Q₇ is hydrogen; amino; C₁-C₆ alkylamino; di C₁-C₆ alkylamino; hydroxyl; C₁-C₆ alkoxy; nitro; cyano; C₁-C₆ alkyl; C₁-C₆ heteroalkyl; C₁-C₆ alkenyl; C₁-C₆ alkynyl; C₃-C₈ cycloalkyl; C₃-C₈ heterocyclyl; aryl; heteroaryl; COR₁₅; SO₂R₁₈; or PO₃R₁₈ or a monosaccharide;

R₁ is CH₂ or CO;

R₃ is hydrogen, halo, C₁-C₆ alkyl, aryl or heteroaryl;

R₁₃ is hydrogen; C₁-C₆ alkyl, C₁-C₆ heteroalkyl, C₃-C₈ cycloalkyl, C₃-C₈ heterocyclyl, aryl, or heteroalkyl each optionally substituted with hydroxyl, C₁-C₆ alkoxy, amino, C₁-C₆ alkylamino, di C₁-C₆ alkylamino; NHCOR₁₅ or COR₁₈;

R₁₅ is hydrogen, hydroxyl, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₁-C₆ alkenyl, C₁-C₆ alkynyl, C₃-C₆ cycloalkyl, C₃-C₆ heterocyclyl, aryl, or heteroaryl;

R₁₈ is hydrogen, hydroxyl, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl; or

a tautomer or an individual isomer or a racemic or non-racemic mixture of isomers, a polymorph, a hydrate, a prodrug or a pharmaceutically acceptable salt or solvate thereof.

In one embodiment, the present invention provides a compound of formula (XV), wherein each Q₂-Q₅ independently is hydrogen, C₁-C₆, alkoxy; halo; amino; or hydroxy; with the proviso that in any compound only one of the Q₃, Q₄ and Q₅ is hydrogen; or a tautomer or an individual isomer or a racemic or non-racemic mixture of isomers, a polymorph, a hydrate, a prodrug or a pharmaceutically acceptable salt or solvate thereof. In one embodiment, the present invention provides a compound of formula (XV), wherein R₁₈ is hydrogen, C₁-C₆ alkyl, C₁-C₆ heteroalkyl, C₁-C₆ alkenyl, C₁-C₆ alkynyl, C₁-C₆ cycloalkyl, C₁-C₆ heterocyclyl, aryl, or heteroaryl; or a tautomer or an individual isomer or a racemic or non-racemic mixture of isomers, a polymorph, a hydrate, a prodrug or a pharmaceutically acceptable salt or solvate thereof.

In one embodiment, the present invention provides a compound of formula (XV), wherein Q₁ is

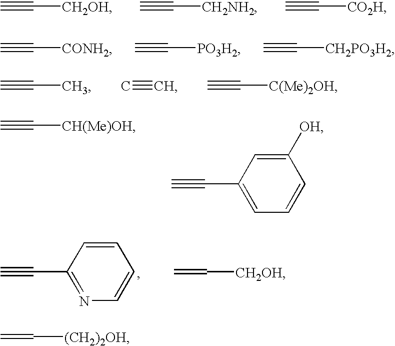

—CH₂—CH₂—OH, —CH₂—CH₂—CH₂—OH, —CONH₂, —CO₂H, —CN, or halo.

In one embodiment, the present invention provides a compound of formula (XV), wherein Q₁ is

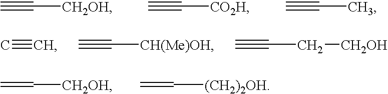

In another aspect, the present invention provides a compound of the formulas (XV-i), (XV-ii) and (XV-iii)

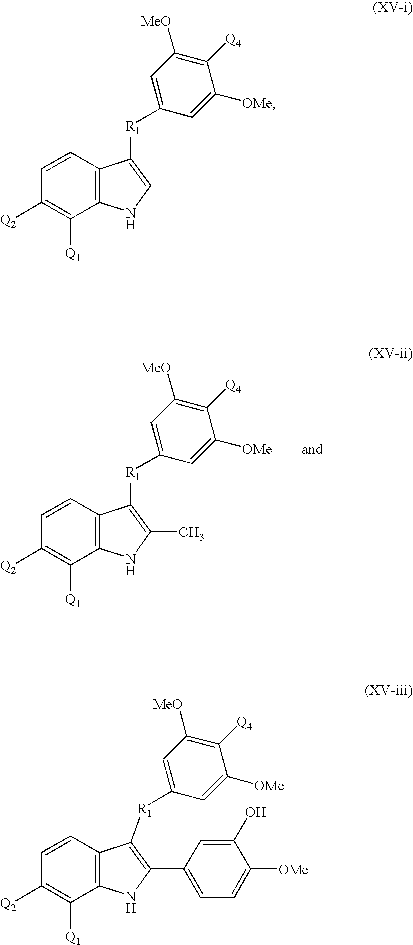

wherein Q₂ is C₁-C₆ alkoxy; and Q₄ is hydrogen or methoxy; or a tautomer or an individual isomer or a racemic or non-racemic mixture of isomers, a polymorph, a hydrate, a prodrug or a pharmaceutically acceptable salt or solvate thereof.

In another aspect, the present invention provides a compound selected from formulas (XVI)-(XX):

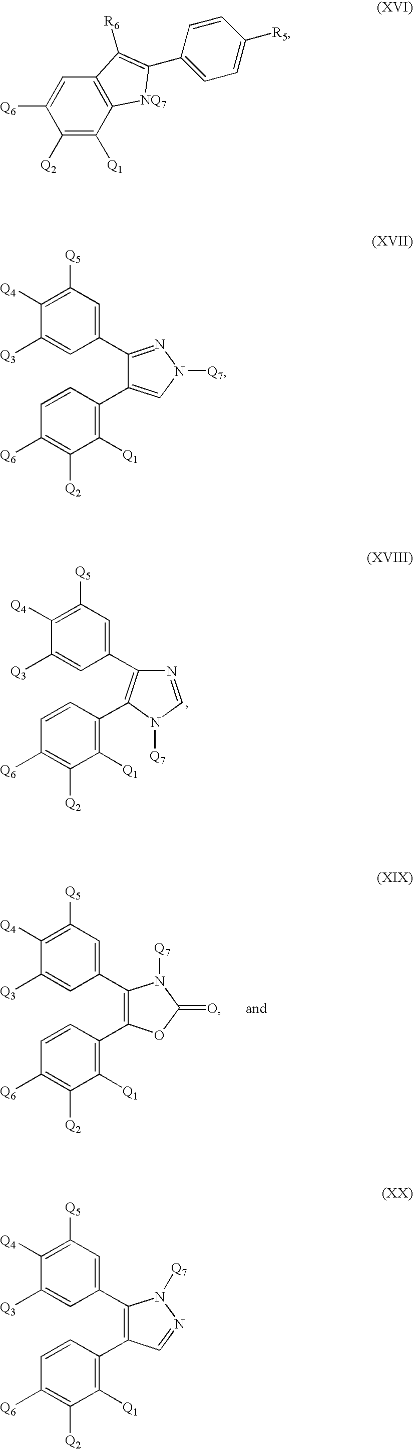

wherein

Q₁ is ≡R₁₃ or

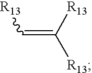

Q₂ is ≡R₁₃;

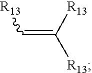

C₁-C₆ alkoxy; halo; amino; or hydroxy;

each Q₃-Q₅ is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈; or PO₃R₁₈ with the proviso that in any one compound, only one of Q₃-Q₅ is hydrogen;

Q₆ is hydrogen; halo; amino; C₁-C₆ alkylamino; di C₁-C₆ alkylamino; hydroxyl; C₁-C₆ alkoxy; nitro; cyano; C₁-C₆ alkyl; C₁-C₆ heteroalkyl; C₁-C₆ alkenyl; C₁-C₆ alkynyl; C₃-C₈ cycloalkyl; C₃-C₈ heterocyclyl; aryl; heteroaryl; COR₁₈; SO₂R₁₈ or PO₃R₁₈;

Q₇ is hydrogen; amino; C₁-C₆ alkylamino; di C₁-C₆ alkylamino; hydroxyl; C₁-C₆ alkoxy; nitro; cyano; C₁-C₆ alkyl; C₁-C₆ heteroalkyl; C₁-C₆ alkenyl; C₁-C₆ alkynyl; C₃-C₈ cycloalkyl; C₃-C₈ heterocyclyl; aryl; heteroaryl; COR₁₅; SO₂R₁₈; or PO₃R₁₈; or a monosaccharide;

R₅ is hydrogen, halo, or C₁-C₆ alkoxy;

R₆ is formyl or a protected form thereof;

R₁₃ is hydrogen; C₁-C₆ alkyl, C₁-C₆ heteroalkyl, C₃-C₈ cycloalkyl, C₃-C₈ heterocyclyl, aryl, or heteroalkyl each optionally substituted with hydroxyl, C₁-C₆ alkoxy, amino, C₁-C₆ alkylamino, di C₁-C₆ alkylamino, NHCOR₁₅ or COR₁₈;

R₁₅ is hydrogen, hydroxyl, C₁-C₆ alkoxy, amino, C₁-C₆ alkylamino, di C₁-C₆alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl;

R₁₈ is hydrogen, C₁-C₆ alkoxy, amino, C₁-C₆alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl; or

a tautomer or an individual isomer or a racemic or non-racemic mixture of isomers, a polymorph, a hydrate, a prod rug or a pharmaceutically acceptable salt or solvate thereof.

In one embodiment, the present invention provides a compound selected from formulas (XVI)-(XX), wherein each Q₂ and Q₆ independently is hydrogen, hydroxy, C₁-C₆ alkoxy, halo, or amino; and each Q₃, Q₄, and Q₅ is OMe. In one embodiment, Q₂ is hydrogen, hydroxyl, fluoro or methoxy; Q₆ is hydrogen, hydroxyl, fluoro, methoxy or amino. In one embodiment, the present invention provides a compound selected from formulas (XVI)-(XX), wherein R₁₈ is hydrogen, C₁-C₆ alkyl, C₁-C₆ heteroalkyl, C₁-C₆ alkenyl, C₁-C₆ alkynyl, C₁-C₆ cycloalkyl, C₁-C₆ heterocyclyl, aryl, or heteroaryl.

In another aspect, the present invention provides a compound of formulas (XXI)-(XXVII):

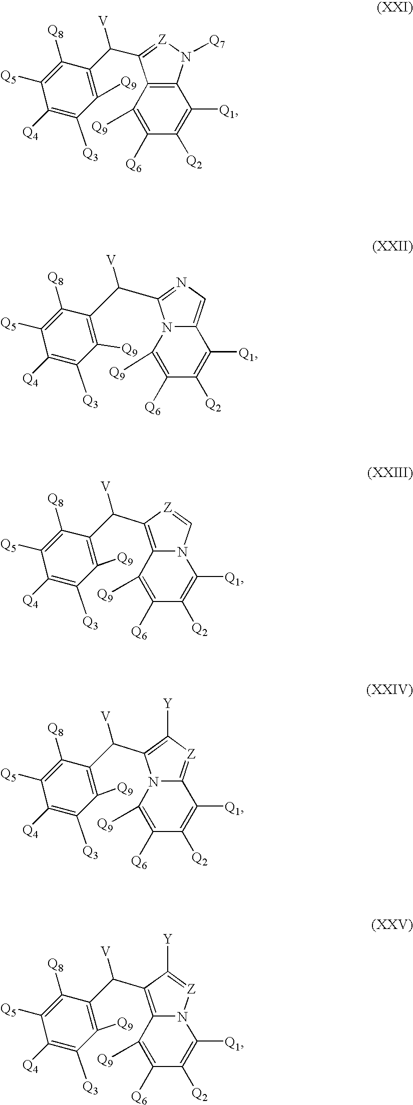

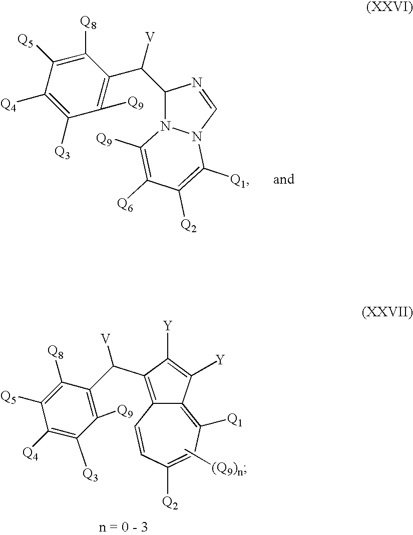

wherein each Q₁, Q₂, and Q₆ independently is hydrogen; halo; amino; C₁-C₆ alkylamino; di C₁-C₆ alkylamino; hydroxyl; C₁-C₆ alkoxy; nitro; cyano; C₁-C₆ alkyl; C₁-C₆ heteroalkyl; C₁-C₆ alkenyl; C₁-C₆ alkynyl; C₃-C₈ cycloalkyl; C₃-C₈ heterocyclyl; aryl; heteroaryl; COR₁₈; SO₂R₁₈ or PO₃R₁₈;

each Q₃-Q₅ is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈; or PO₃R₁₈; Q₃ and Q₄ together form C₃-C₈ heterocycle, an aryl, or a heteroaryl; or Q₄ and Q₅ together form a C₃-C₈ heterocycle, an aryl, or a heteroaryl; with the proviso that in any one compound, only one of Q₃-Q₅ is hydrogen;

Q₇ is hydrogen; halo; amino; C₁-C₆ alkylamino; di C₁-C₆ alkylamino; hydroxyl; C₁-C₆ alkoxy; nitro; cyano; C₁-C₆ alkyl; C₁-C₆ heteroalkyl; C₁-C₆ alkenyl; C₁-C₆ alkynyl; C₃-C₈ cycloalkyl; C₃-C₈ heterocyclyl; aryl; heteroaryl; COR₁₅; SO₂R₁₈; or PO₃R₁₈ or a monosaccharide; with the proviso that in formula (II) Q₇ excludes hydrogen;

Q₈ is hydrogen; halo; amino; C₁-C₆ alkylamino; di C₁-C₆ alkylamino; hydroxyl; C₁-C₆ alkoxy; nitro; cyano; aryl; heteroaryl; COR₁₅; SO₂R₁₅ or PO₃R₁₅;

each Q₉ independently is hydrogen; halo; amino; C₁-C₆ alkylamino; di C₁-C₆ alkylamino; hydroxyl; C₁-C₆ alkoxy; nitro; cyano; aryl; heteroaryl; COR₁₅; SO₂R₁₅ or PO₃R₁₅;

V is —NHR₁₆; —NHNHR₁₆; —NHN(R₁₆)₂; —NR₁₆NHR₁₆; or —OR₁₇;

Y is hydrogen, hydroxyl or halogen;

Z is —CH— or —N—;

R₁₅ is hydrogen, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₁-C₆ alkenyl, C₁-C₆ alkynyl, C₁-C₆ cycloalkyl, C₁-C₆ heterocyclyl, aryl, or heteroaryl;

R₁₆ is hydrogen, C₁-C₆alkyl, aryl, C₁-C₆alkylsulphonyl, arylsulfonyl, C₁-C₆ alkoxycarbonyl, aminocarbonyl, C₁-C₆ alkylaminocarbonyl, di C₁-C₆ alkylaminocarbonyl, C₁-C₆ acyl, aroyl, aminothiocarbonyl, C₁-C₆ alkylaminothiocarbonyl, di C₁-C₆ alkylaminothiocarbonyl, C₁-C₆ thioacyl, or thioaroyl; and R′ is C₁-C₆ alkyl or aryl; with the proviso that when V is NR₁₆, R₁₆ excludes hydrogen;

R₁₇ is C₁-C₆ alkyl; aryl; or di C₁-C₆ alkylamino;

R₁₈ is hydrogen, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl; or

a tautomer or an individual isomer or a racemic or non-racemic mixture of isomers, a polymorph, a hydrate, a prod rug or a pharmaceutically acceptable salt or solvate thereof.

In one embodiment, the present invention provides a compound of formula (XXI)-(XXVII), wherein Q₁ is hydrogen; halo; cyano; nitro; COR₁₈; SO₂R₁₈; PO₃R₁₈; ≡R₁₃ or

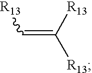

### Q₂ is ≡R₁₃;

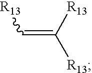

C₁-C₆ alkoxy; halo; amino; or hydroxy; each Q₃, Q₄ and Q₅ independently is hydrogen, C₁-C₆ alkoxy, halo, amino, or hydroxyl provided that in any compound only one of the Q₃, Q₄ and Q₅ is hydrogen; Q₇ is C₁-C₆ alkyl optionally substituted independently with one or more aryl, heteroaryl, hydroxyl, amino, C₁-C₆alkylamino, di C₁-C₆alkylamino, CO₂H, or CONH₂; COR₁₈; SO₂R₁₈; or PO₃R₁₈; or a monosaccharide;

R₁₃ is hydrogen; C₁-C₆ alkyl, C₁-C₆ heteroalkyl, C₃-C₈ cycloalkyl, C₃-C₈ heterocyclyl, aryl, or heteroalkyl each optionally substituted with hydroxyl, C₁-C₆ alkoxy, amino, C₁-C₆ alkylamino, di C₁-C₆ alkylamino; NHCOR₁₅ or COR₁₈;

R₁₅ is hydrogen, hydroxyl, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₁-C₆ alkenyl, C₁-C₆ alkynyl, C₃-C₆ cycloalkyl, C₃-C₆ heterocyclyl, aryl, or heteroaryl;

R₁₆ is hydrogen, C₁-C₆ alkoxy, amino, C₁-C₆ alkylamino, di C₁-C₆ alkylamino, NHOH, or NHNH₂, C₁-C₆ alkyl, C₁-C₆ heteroalkyl, C₃-C₈ cycloalkyl, C₃-C₈ heterocyclyl, aryl, or heteroalkyl, NHOH, NHNH₂, and

R₁₈ is hydrogen, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl; or

a tautomer or an individual isomer or a racemic or non-racemic mixture of isomers, a polymorph, a hydrate, a prodrug or a pharmaceutically acceptable salt or solvate thereof.

In one embodiment, the present invention provides a compound of formulas (XXI-i), (XXII-i), (XXIII-i), (XXIV-i), (XXV-i) and (XXVII-i):

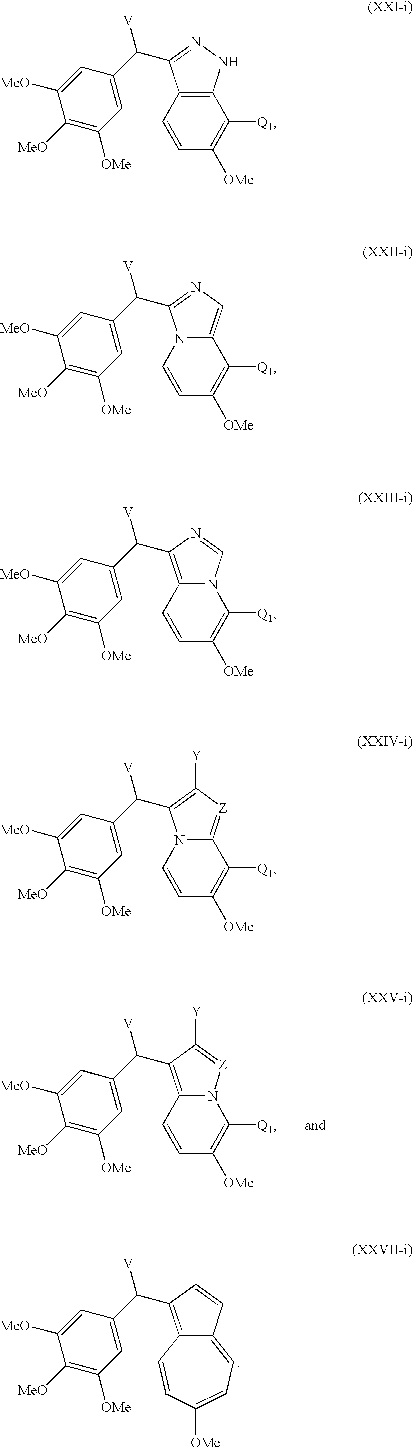

wherein Q₁, V, Y, and Z is defined as above; or a tautomer or an individual isomer or a racemic or non-racemic mixture of isomers, a polymorph, a hydrate, a prodrug or a pharmaceutically acceptable salt or solvate thereof.

In another aspect, the present invention provides prodrug compounds as defined above wherein the tubulin binding compound is bonded to the hypoxic activator (Hyp) through an hydroxyloxygen (-OHyp) or an amine nitrogen

(-NHyp) in the tubulin binding compound. The hypoxic activator can be electron deficient nitrobenzene moieties, electron deficient nitrobenzoic acid amide moieties, nitroazole moieties, nitroimidazole moieties, nitrothiophene moieties, nitrothiazole moieties, nitrooxazole moieties, nitrofuran moieties, and nitropyrrole moieties. In one embodiment, the hypoxic activator is a substituted or unsubstituted nitroimidazole moiety.

In one embodiment, Hyp is selected from:

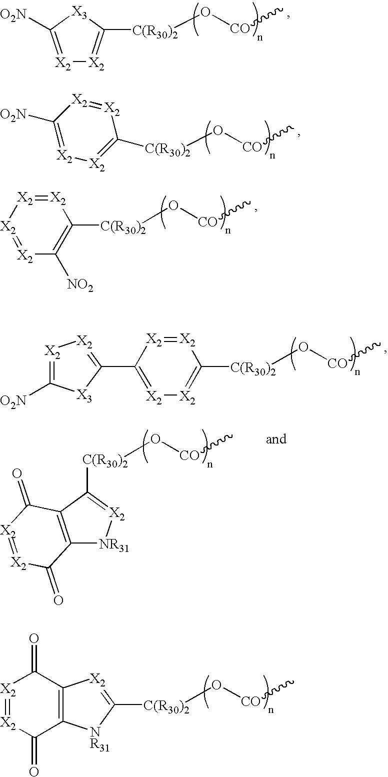

wherein each X₂ is N or CR₃₂;

X₃ is NR₃₁, S, or O;

each R₃₀ is independently hydrogen or alkyl;

R₃₁ is hydrogen, hydroxyl, C₁-C₆ alkyl or heteroalkyl, C₃-C₈ cycloalkyl, heterocyclyl, C₁-C₆ alkoxy, C₁-C₆ alkylamino, C₁-C₆ dialkylamino, aryl or heteroaryl, C₁-C₆ acyl or heteroacyl, aroyl, or heteroaroyl;

R₃₂ is hydrogen, halogen, nitro, cyano, CO₂H, C₁-C₆ alkyl or heteroalkyl, C₁-C₆ cycloalkyl, C₁-C₆ alkoxy, C₁-C₆ alkylamino, C₁-C₆ dialkylamino, aryl, CON(R₇)₂, C₁-C₆ acyl or heteroacyl, or aroyl or heteroaroyl; and

n=0, 1.

In an additional embodiment, Hyp is selected from

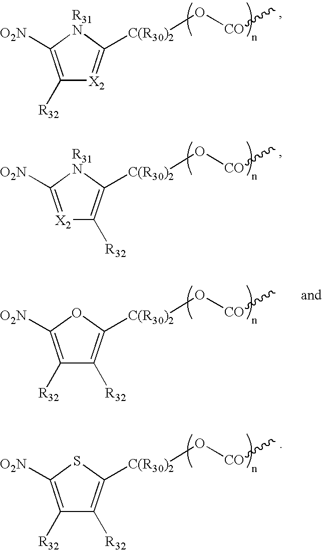

wherein X₂, R₃₀, R₃₁, R₃₂ and n are as defined above.

In another embodiment, Hyp is

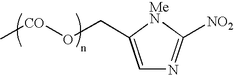

wherein n=0 or 1, provided that in -OHyp n=0.

In one embodiment, the present invention provides compounds of the invention wherein X is —NN(Hyp)R wherein Hyp and R are defined as above.

In another embodiment, the present invention provides a prodrug of the compound of formula (I-i):

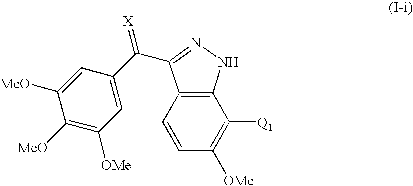

wherein one or more —NH—, enol form of a C═O, and/or —OH moiety or moieties therein is converted to

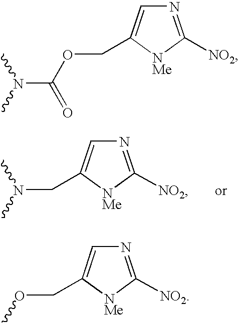

In one embodiment, the present invention provides a compound of formulas (XXVIII)-(XXXII):

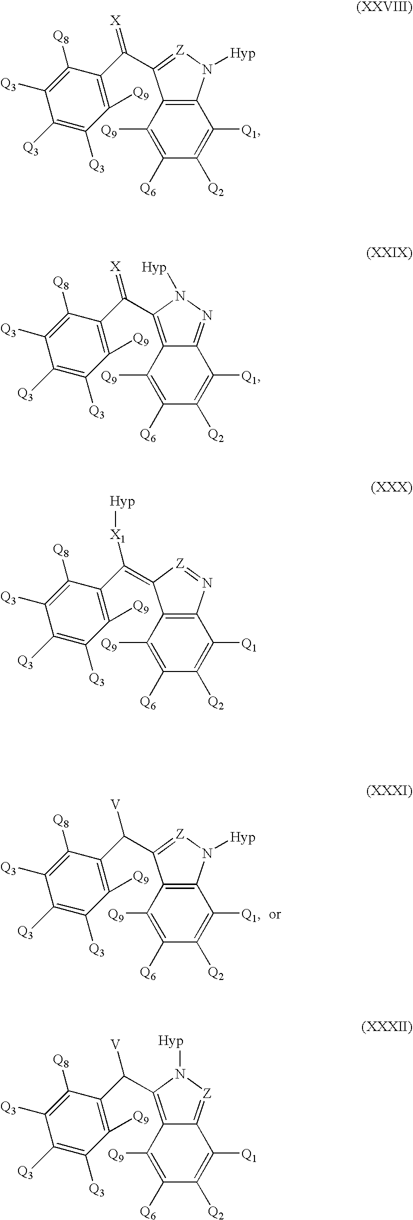

wherein each Q₁, Q₂, and Q₆ independently is hydrogen; halo; amino; C₁-C₆ alkylamino; di C₁-C₆ alkylamino; hydroxyl; C₁-C₆ alkoxy; nitro; cyano; C₁-C₆ alkyl; C₁-C₆ heteroalkyl; C₂-C₆ alkenyl; C₂-C₆ alkynyl; C₃-C₈ cycloalkyl; C₃-C₈ heterocyclyl; aryl; heteroaryl; COR₁₈; SO₂R₁₈; or PO₃R₁₈;

each Q₃-Q₅ is hydrogen; halo; amino; C₁-C₆ alkylamino; di C₁₋₆ alkylamino; hydroxyl; C₁-C₆ alkoxy; nitro; cyano; aryl; heteroaryl; COR₁₈; SO₂R₁₈; or PO₃R₁₈; Q₃ and Q₄ together form C₃-C₈ heterocycle, an aryl, or a heteroaryl; or Q₄ and Q₅ together form a C₃-C₈ heterocycle, an aryl, or a heteroaryl; with the proviso that in any one compound, only one of Q₃-Q₅ is hydrogen;

Q₈ is hydrogen; halo; amino; C₁-C₆ alkylamino; di C₁-C₆ alkylamino; hydroxyl; C₁-C₆ alkoxy; nitro; cyano; aryl; heteroaryl; COR₁₅; SO₂R₁₅ or PO₃R₁₅;

each Q₉ independently is hydrogen; halo; amino; C₁-C₆ alkylamino; di C₁-C₆ alkylamino; hydroxyl; C₁-C₆ alkoxy; nitro; cyano; aryl; heteroaryl; COR₁₅; SO₂R₁₅ or PO₃R₁₅;

V is —NHNHR₁₆; —HNR₁₆; —N(Hyp)NHR₁₆; —NHN(Hyp)R₁₆; or

—N(Hyp)N(Hyp)R; wherein Hyp is a hypoxic activator as defined above;

X is O, —NNHR₁₆, NR₁₆, —NN(Hyp)R₁₆, or NOR₁₆ wherein R₁₆ is C₁-C₆ alkyl, aryl, C₁-C₆ alkylsulphonyl, arylsulfonyl, C₁-C₆ alkoxycarbony, aminocarbonyl, C₁-C₆ alkylaminocarbonyl, di C₁-C₆ alkylaminocarbonyl, C₁-C₆ acyl, aroyl, aminothiocarbonyl, C₁-C₆alkylaminothiocarbonyl, di C₁-C₆ alkylaminothiocarbonyl, C₁-C₆ thioacyl, or thioaroyl; with the proviso that when X is NR₁₆, R₁₆ excludes hydrogen;

Y is hydrogen, hydroxyl, or halogen;

Z is —CH— or —N—;

R₁₅ is hydrogen, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl;

R₁₆ is hydrogen, C₁-C₆alkyl, aryl, C₁-C₆alkylsulphonyl, arylsulfonyl, C₁-C₆ alkoxycarbonyl, aminocarbonyl, C₁-C₆ alkylaminocarbonyl, di C₁-C₆ alkylaminocarbonyl, C₁-C₆ acyl, aroyl, aminothiocarbonyl, C₁-C₆ alkylaminothiocarbonyl, di C₁-C₆ alkylaminothiocarbonyl, C₁-C₆ thioacyl, or thioaroyl; with the proviso that when X is NR₁₆, R₁₆ excludes hydrogen;

R₁₈ is hydrogen, hydroxyl, C₁-C₆ alkoxy, amino, C₁-C₆alkylamino, di C₁-C₆alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl; or

a tautomer or an individual isomer or a racemic or non-racemic mixture of isomers, a polymorph, a hydrate, a prodrug or a pharmaceutically acceptable salt or solvate thereof.

In one embodiment, the present invention provides a compound of formulas (XXVIII)-(XXXII), wherein Q₁ is hydrogen; halo; cyano; nitro; COR₁₅; SO₂R₁₅; PO₃R₁₅; ≡R₁₃ or

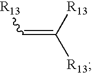

### Q₂ is ≡R₁₃,

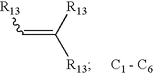

alkoxy; halo; amino; or hydroxy; each Q₃, Q₄ and Q₅ independently is hydrogen, C₁-C₆ alkoxy, halo, amino, or hydroxylprovided that in any compound only one of the Q3, Q4, and Q₅ is hydrogen;

R₁₃ is hydrogen; C₁-C₆ alkyl, C₁-C₆ heteroalkyl, C₃-C₈ cycloalkyl, C₃-C₈ heterocyclyl, aryl, or heteroalkyl each optionally substituted with hydroxyl, C₁-C₆ alkoxy, amino, C₁-C₆ alkylamino, di C₁-C₆ alkylamino; NHCOR₁₅ or COR₁₈; or a tautomer or an individual isomer or a racemic or non-racemic mixture of isomers, a polymorph, a hydrate, a prodrug or a pharmaceutically acceptable salt or solvate thereof.

In one embodiment, the present invention provides a compound of formula:

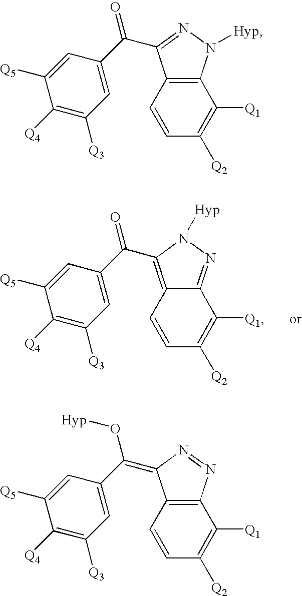

wherein Q₁ is hydrogen,

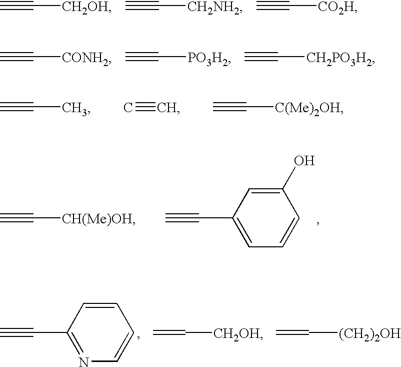

—CH₂—CH₂—OH, —CH₂—CH₂—CH₂—OH, —CONH₂, —CO₂H, —CN, or halo; or a tautomer or an individual isomer or a racemic or non-racemic mixture of isomers, a polymorph, a hydrate, a prodrug or a pharmaceutically acceptable salt or solvate thereof.

In one embodiment, Q₁ is

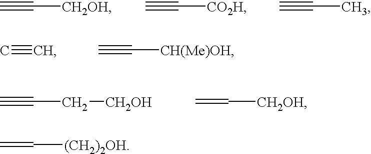

In one embodiment, the present invention provides a compound of selected from the group consisting of:

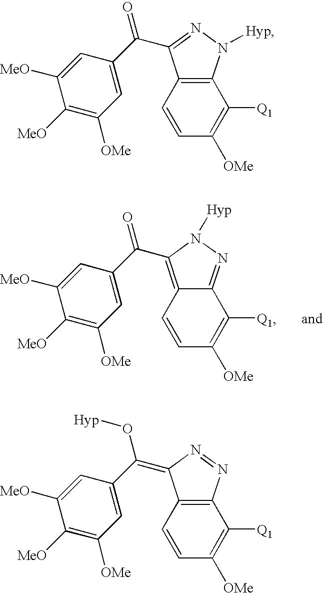

wherein Hyp is as defined above.

In one embodiment, the present invention provides a compound of formula:

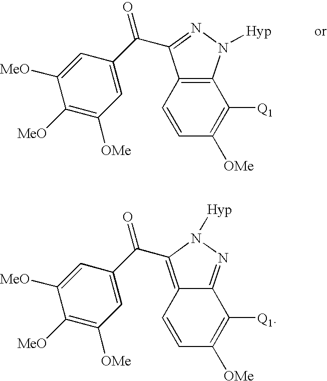

wherein Hyp is as defined above.

In another embodiment, the present invention provides a compound of formula (XIV):

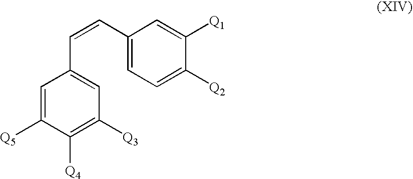

wherein; each Q₃-Q₅ is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈, or PO₃R₁₈; Q₃ and Q₄ together form C₃-C₈ heterocycle, an aryl, or a heteroaryl; or Q₄ and Q₅ together form a C₃-C₈ heterocycle, an aryl, or a heteroaryl; (-OHyp) or (-NHyp) with the proviso that in any one compound, at least one of Q₃-Q₅ is (-OHyp) or (-NHyp);

Q₁, Q₂, R₁₃, R₁₅ and Hyp are as defined above; or

a tautomer or an individual isomer or a racemic or non-racemic mixture of isomers, a polymorph, a hydrate, a prodrug or a pharmaceutically acceptable salt or solvate thereof. In one embodiment, the present invention provides a compound of formula (XIV), wherein Q₁ is R₁₃; or

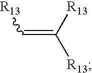

and each Q₂-Q₅ independently is hydrogen, C₁-C₆ alkoxy; halo; amino; or hydroxy; with the proviso that in any compound at least one of Q₃-Q₅ is (-OHyp) or (-NHyp); or a tautomer or an individual isomer or a racemic or non-racemic mixture of isomers, a polymorph, a hydrate, a prodrug or a pharmaceutically acceptable salt or solvate thereof.

In one embodiment, the present invention provides a compound of formula (XXXIV):

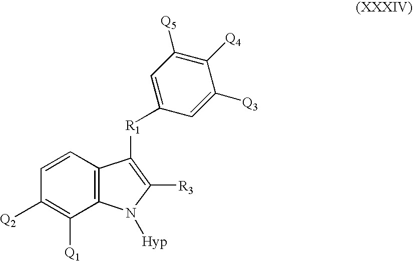

wherein Q₁ is ≡R₁₃ or

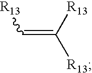

Q₂ is ≡R₁₃

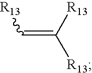

C₁-C₆ alkoxy; halo; amino; or hydroxy;

each Q₃, Q₄, and Q₅ independently is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈; or PO₃R₁₈; Q₃ and Q₄ together form C₃-C₈ heterocycle, an aryl, or a heteroaryl; or Q₄ and Q₅ together form a C₃-C₈ heterocycle, an aryl, or a heteroaryl; with the proviso that in any one compound, only one of Q₃-Q₅ is hydrogen;

R₁ is CH₂ or CO;

R₃ is hydrogen, halo, C₁-C₆ alkyl, aryl or heteroaryl;

R₁₃ is hydrogen; C₁-C₆ alkyl, C₁-C₆ heteroalkyl, C₃-C₈ cycloalkyl, C₃-C₈ heterocyclyl, aryl, or heteroalkyl each optionally substituted with hydroxyl, C₁-C₆ alkoxy, amino, C₁-C₆ alkylamino, di C₁-C₆ alkylamino; NHCOR₁₅ or COR₁₈

R₁₅ is hydrogen, hydroxyl, C₁-C₆ alkoxy, amino, C₁-C₆alkylamino, di C₁-C₆ alkylamino, NHOH, NHNH₂, C₁-C₆ alkyl, C₁-C₆ heteroalkyl, C₁-C₆ alkenyl, C₁-C₆ alkynyl, C₃-C₆ cycloalkyl, C₃-C₆ heterocyclyl, aryl, or heteroaryl;

R₁₈ is hydrogen, hydroxyl, C₁-C₆ alkoxy, amino, C₁-C₆alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl;

Hyp is hypoxic activator; or

a tautomer or an individual isomer or a racemic or non-racemic mixture of isomers, a polymorph, a hydrate, a prodrug or a pharmaceutically acceptable salt or solvate thereof.

In one embodiment, the present invention provides a compound of formula (XXXIV-i), (XXXIV-ii) and (XXXIV-iii)

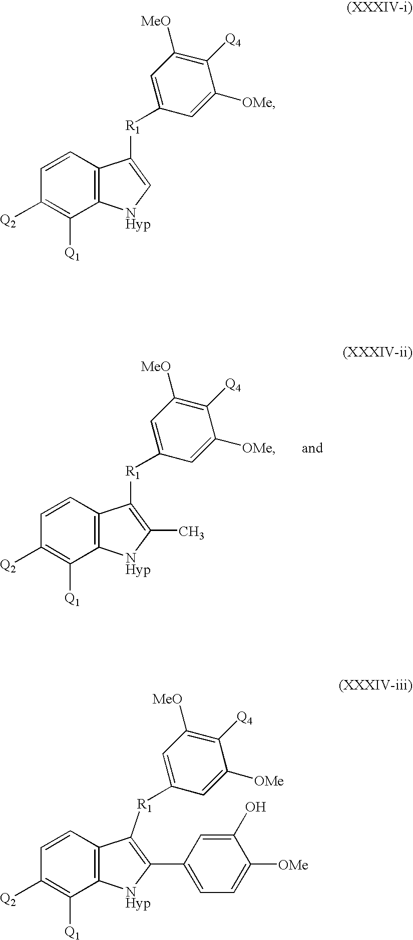

wherein Q₂ is C₁-C₆ alkoxy and Q₄ is hydrogen or methoxy; or a tautomer or an individual isomer or a racemic or non-racemic mixture of isomers, a polymorph, a hydrate, a prodrug or a pharmaceutically acceptable salt or solvate thereof.

In one embodiment, the present invention provides a compound of formulas (XXXV)-(XXXIX):

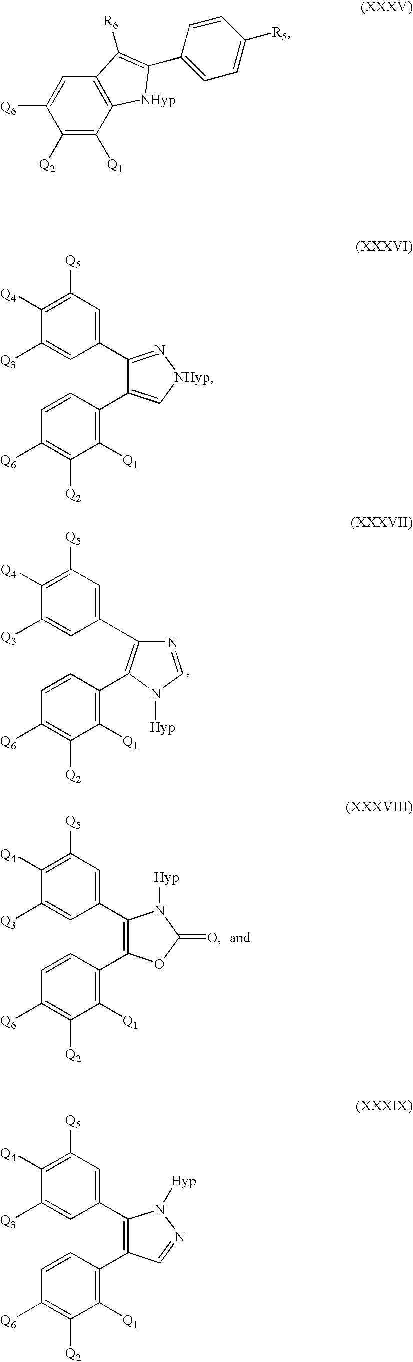

wherein

Q₁ is ≡R₁₃ or

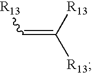

Q₂ is ≡R₁₃;

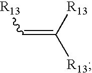

C₁-C₆ alkoxy; halo; amino; or hydroxy;

each Q₃-Q₅ is hydrogen; halo; amino; C₁-C₆ alkylamino; di C₁-C₆ alkylamino; hydroxyl; C₁-C₆ alkoxy; nitro; cyano; heteroaryl; COR₁₅; SO₂R₁₅; or PO₃R₁₅; Q₃ and Q₄ together form C₃-C₈ heterocycle, an aryl, or a heteroaryl; or Q₄ and Q₅ together form a C₃-C₈ heterocycle, an aryl, or a heteroaryl; with the proviso that in any one compound, only one of Q₃-Q₅ is hydrogen;

Q₆ is hydrogen; halo; amino; C₁-C₆ alkylamino; di C₁-C₆ alkylamino; hydroxyl; C₁-C₆ alkoxy; nitro; cyano; C₁-C₆ alkyl; C₁-C₆ heteroalkyl; C₁-C₆ alkenyl; C₁-C₆ alkynyl; C₃-C₈ cycloalkyl; C₃-C₈ heterocyclyl; aryl; heteroaryl; COR₁₈; SO₂R₁₈ or PO₃R₁₈;

Q₇ is hydrogen; amino; C₁-C₆ alkylamino; di C₁-C₆ alkylamino; hydroxyl; C₁-C₆ alkoxy; nitro; cyano; C₁-C₆ alkyl; C₁-C₆ heteroalkyl; C₁-C₆ alkenyl; C₁-C₆ alkynyl; C₃-C₈ cycloalkyl; C₃-C₈ heterocyclyl; aryl; heteroaryl; COR₁₅; SO₂R₁₈; or PO₃R₁₈; or a monosaccharide;

R₅ is hydrogen, halo, or C₁-C₆ alkoxy;

R₆ is formyl or a protected form thereof;

R₁₃ is hydrogen; C₁-C₆ alkyl, C₁-C₆ heteroalkyl, C₃-C₈ cycloalkyl, C₃-C₈ heterocyclyl, aryl, or heteroalkyl each optionally substituted with hydroxyl, C₁-C₆ alkoxy, amino, C₁-C₆ alkylamino, di C₁-C₆ alkylamino, NHCOR₁₅ or COR₁₅;

R₁₅ is hydrogen, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl;

R₁₈ is hydrogen, hydroxyl, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl;

Hyp is hypoxic activator; or

a tautomer or an individual isomer or a racemic or non-racemic mixture of isomers, a polymorph, a hydrate, a prodrug or a pharmaceutically acceptable salt or solvate thereof.

In another embodiment, the present invention provides prodrug compounds selected from the group consisting of:

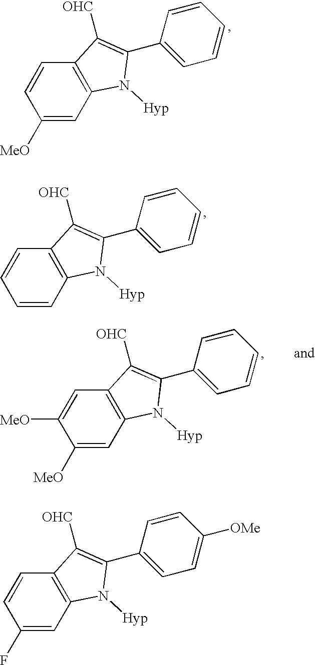

and a tautomer or an individual isomer or a racemic or non-racemic mixture of isomers, a polymorph, a hydrate, a prodrug or a pharmaceutically acceptable salt or solvate thereof.

In one embodiment, the present invention provides a compound of formulas (XXXV)-(XXXIX), wherein each Q₂ and Q₆ independently is hydrogen, hydroxy, C₁-C₆ alkoxy, halo, or amino; and each Q₃, Q₄, and Q₅ is OMe. In one embodiment, Q₂ is hydrogen, hydroxyl, fluoro or methoxy; Q₆ is hydrogen, hydroxyl, fluoro, methoxy or amino; or a tautomer or an individual isomer or a racemic or non-racemic mixture of isomers, a polymorph, a hydrate, a prodrug or a pharmaceutically acceptable salt or solvate thereof. In one embodiment, R₁₈ is hydrogen, C₁-C₆ alkyl, C₁-C₆ heteroalkyl, C₁-C₆ alkenyl, C₁-C₆ alkynyl, C₁-C₆ cycloalkyl, C₁-C₆ heterocyclyl, aryl, or heteroaryl; or a tautomer or an individual isomer or a racemic or non-racemic mixture of isomers, a polymorph, a hydrate, a prodrug or a pharmaceutically acceptable salt or solvate thereof.

In one embodiment, the present invention provides a compound of formulas (XL)-(XLIII)

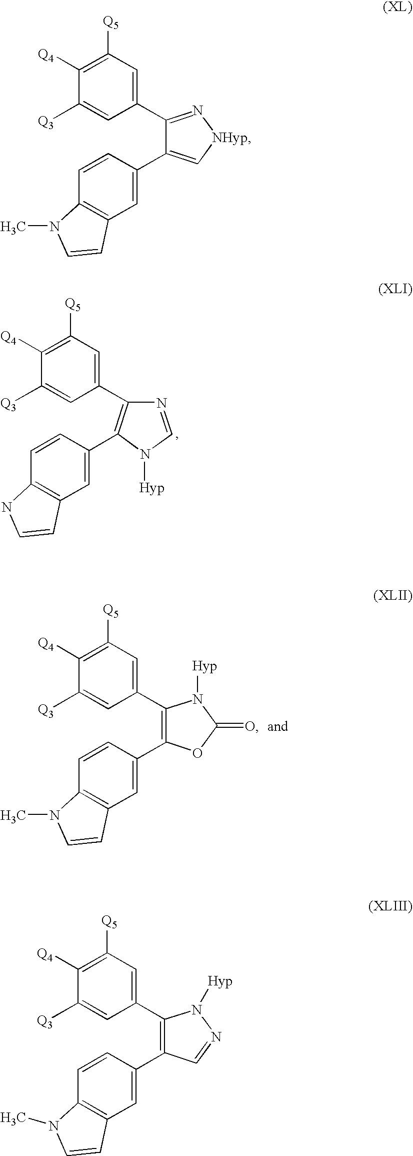

wherein

each Q₃-Q₅ is hydrogen; halo; amino; C₁-C₆ alkylamino; di C₁-C₆ alkylamino; hydroxyl; C₁-C₆ alkoxy; nitro; cyano; aryl; heteroaryl; COR₁₅; SO₂R₁₅; or PO₃R₁₅ with the proviso that in any one compound, only one of Q₃-Q₅ is hydrogen;

R₁₅ is hydrogen, hydroxyl, C₁-C₆ alkoxy, amino, C₁-C₆ alkylamino, di C₁-C₆alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl;

Hyp is hypoxic activator; or

a tautomer or an individual isomer or a racemic or non-racemic mixture of isomers, a polymorph, a hydrate, a prodrug or a pharmaceutically acceptable salt or solvate thereof.

In one embodiment, the present invention provides a compound of formula (XLIV):

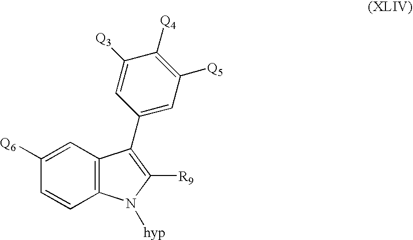

each Q₃-Q₅ is hydrogen; halo; amino; C₁-C₆ alkylamino; di C₁-C₆ alkylamino; hydroxyl; C₁-C₆ alkoxy; nitro; cyano; aryl; heteroaryl; COR₁₅; SO₂R₁₅; or PO₃R₁₅ with the proviso that in any one compound, only one of Q₃-Q₅ is hydrogen;

R₉ is C₁-C₆ alkyl; aryl; or heteroaryl;

R₁₅ is hydrogen, hydroxyl, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl; or

a tautomer or an individual isomer or a racemic or non-racemic mixture of isomers, a polymorph, a hydrate, a prodrug or a pharmaceutically acceptable salt or solvate thereof. In one embodiment, in the compound of formula (XLIV), R₉ is:

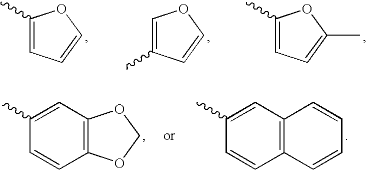

In one embodiment, the present invention provides a compound of formula (XLV):

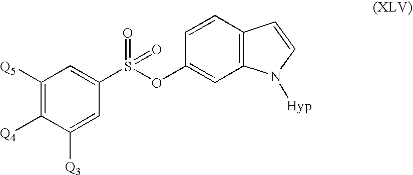

wherein each Q₃-Q₅ is hydrogen; halo; amino; C₁-C₆ alkylamino; di C₁-C₆ alkylamino; hydroxyl; C₁-C₆ alkoxy; nitro; cyano; aryl; heteroaryl; COR₁₅; SO₂R₁₅; or PO₃R₁₅; Q₃ and Q₄ together form C₃-C₈ heterocycle, an aryl, or a heteroaryl; or Q₄ and Q₅ together form a C₃-C₈ heterocycle, an aryl, or a heteroaryl; with the proviso that in any one compound, only one of Q₃-Q₅ is hydrogen;

R₁₅ is hydrogen, hydroxyl, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl;

Hyp is hypoxic activator; or

a tautomer or an individual isomer or a racemic or non-racemic mixture of isomers, a polymorph, a hydrate, a prodrug or a pharmaceutically acceptable salt or solvate thereof. In one embodiment, each Q₃-Q₅ is OMe.

In one embodiment, the present invention provides a compound of formula (XLVI):

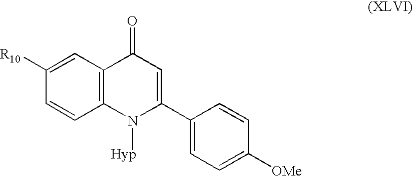

wherein R₁₀ is C₁-C₆ alkyl and Hyp is hypoxic activator; and a tautomer or an individual isomer or a racemic or non-racemic mixture of isomers, a polymorph, a hydrate, a prodrug or a pharmaceutically acceptable salt or solvate thereof. In one embodiment, R₁₀ is methyl.

In another embodiment, the present invention provides the prodrug compound of formula (XLVII):

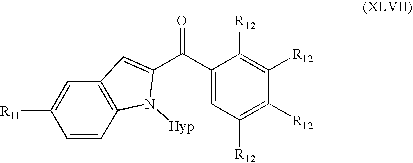

wherein R₁₁ is methoxy or methyl and each R₁₂ is halogen, methoxy, methyl, nitro, or amino; Hyp is defined as above; and a tautomer or an individual isomer or a racemic or non-racemic mixture of isomers, a polymorph, a hydrate, a prodrug or a pharmaceutically acceptable salt or solvate thereof.

In one embodiment, the present invention provides a compound of formula (I)-(XLVII), wherein Q₁ is

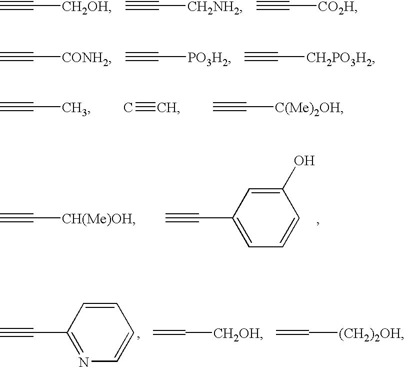

—CH₂—CH₂—OH, —CH₂—CH₂—CH₂—OH, —CONH₂, —CO₂H, —CN, or halo.

In one embodiment, the present invention provides a compound of formula (I)-(XLVII), wherein Q₁ is

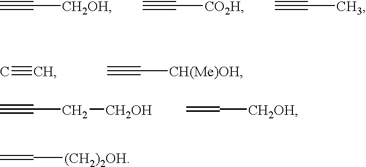

In addition to compounds having formulas (I)-(XLVII) above, the present invention further includes all salts thereof, and particularly, pharmaceutically acceptable salts thereof. Still further, the invention includes compounds that are single isomers of the above formula (e.g., single enantiomers of compounds having a single chiral center), as well as solvate, hydrate, a prodrug and tautomeric forms thereof. In other embodiments isomers include single geometric isomers such as cis, trans, E and Z forms of compounds with geometric isomers, or single tautomers of compounds having two or more tautomers.

In another embodiment, the present invention provides prodrug compounds selected from the group consisting of:

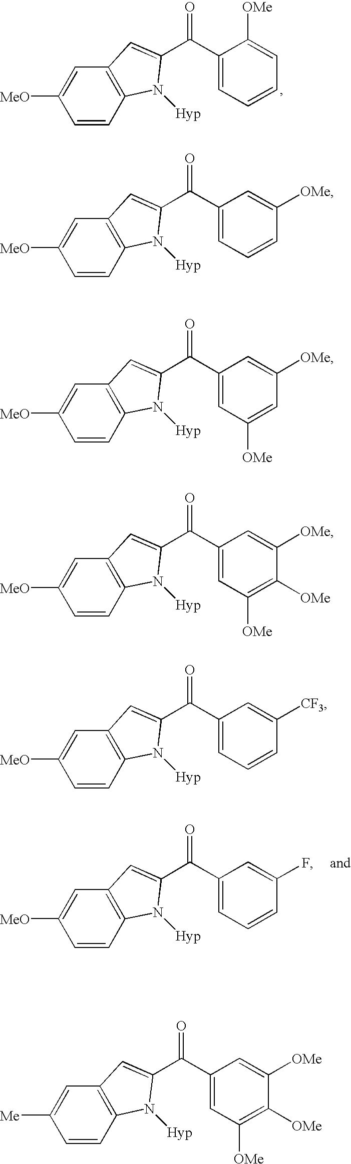

Hyp is defined as above; and a tautomer or an individual isomer or a racemic or non-racemic mixture of isomers, a polymorph, a hydrate, a prodrug or a pharmaceutically acceptable salt or solvate thereof. In one embodiment the present invention provides the following compounds:

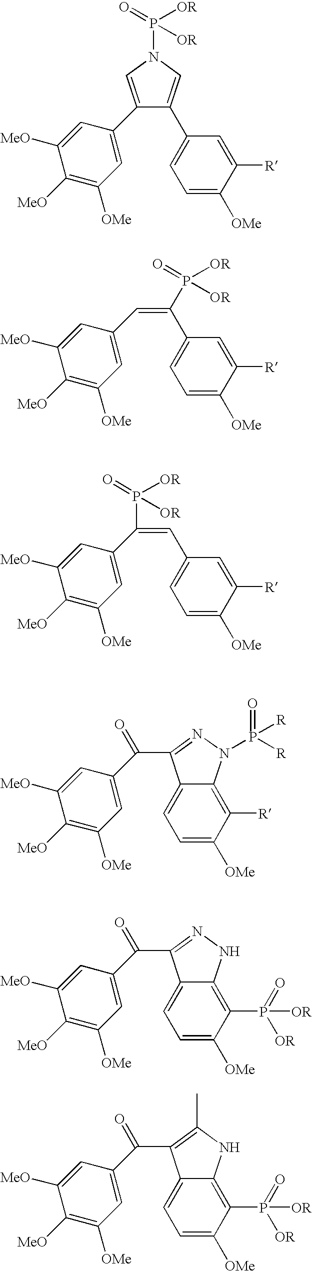

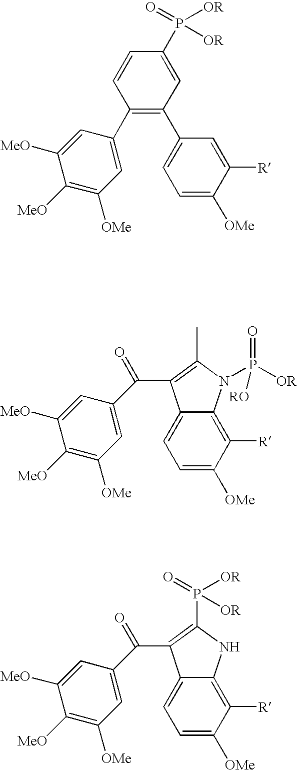

wherein R is a group which undergoes a tumor specific release such as triggering under hypoxic conditions and R′═NH₂, OH, Cl, F, and Br

In one embodiment, the present invention provides novel prodrug compounds of the following tubulin binders:

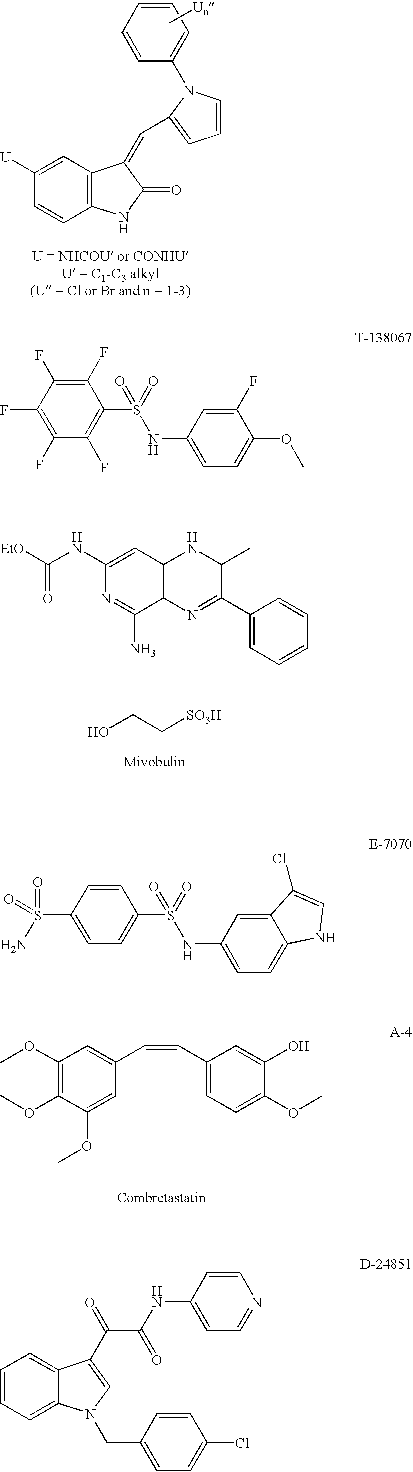

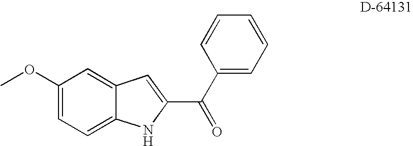

wherein each —NH— or OH moiety in a structure above is replaced with —N(Hyp)- wherein Hyp is defined as above. In another embodiment, one —NH-moiety in each structure is replaced with —N(Hyp)-. In one embodiment, where a structure has more than one —NH— moiety, two of those are replaced with —N(Hyp)-.

In another aspect, the present invention provides a pharmaceutical composition comprising a pharmaceutically acceptable carrier and a novel compound or a novel prodrug compound of the invention.

In another aspect, the present invention provides a method of treating cancer comprising administering a therapeutically effective amount of a novel compound or a novel prodrug compound of the invention alone or in combination with one or more other anti-cancer agents to a subject in need of such treatment. In another aspect, the present invention provides a method of treating a hyperproliferative disease comprising administering a therapeutically effective amount of a novel compound or a novel prodrug compound of the invention to a subject in need of such treatment.

In one embodiment, examples of compounds of the present invention include but are not limited to the following compounds:

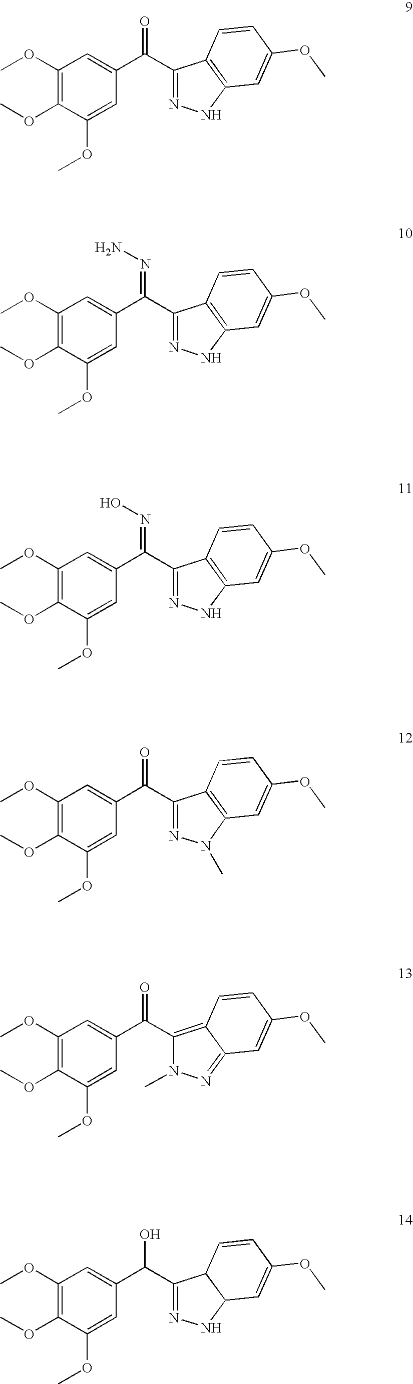

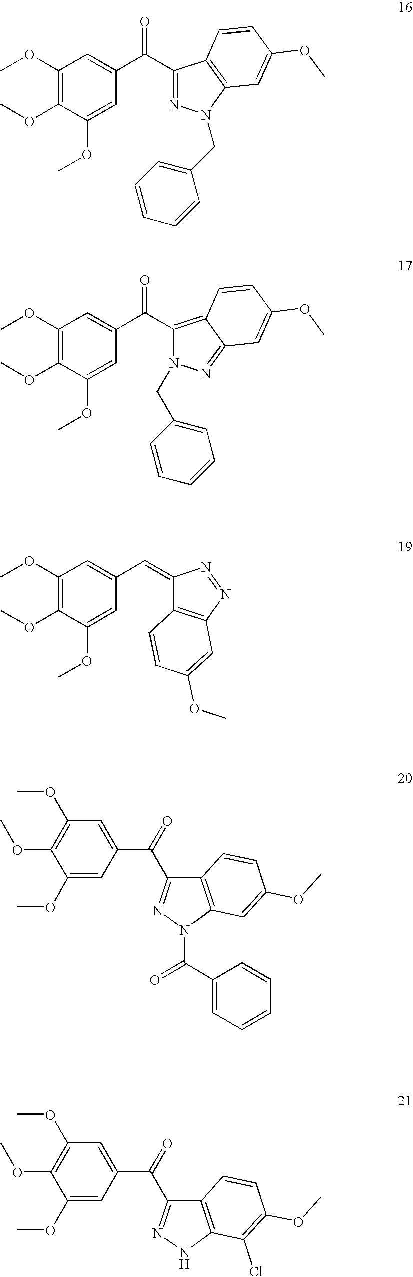

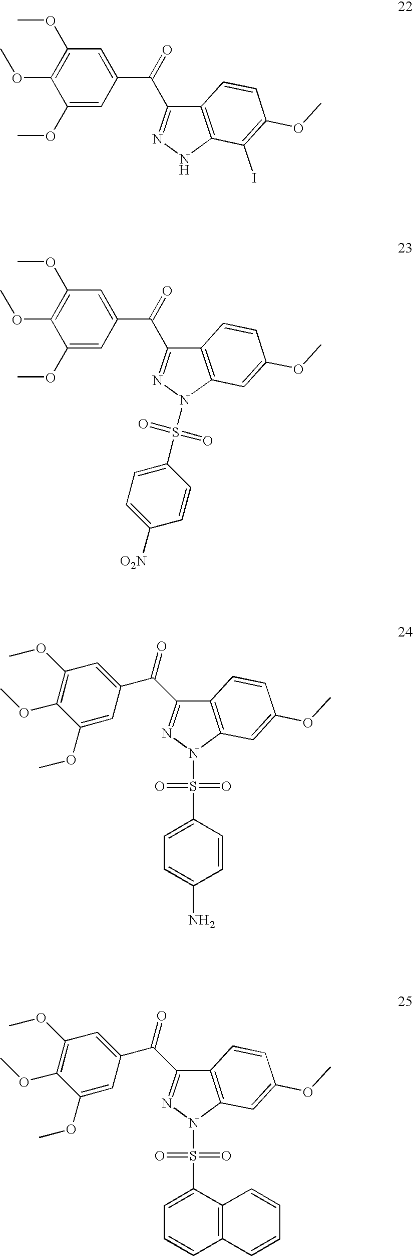

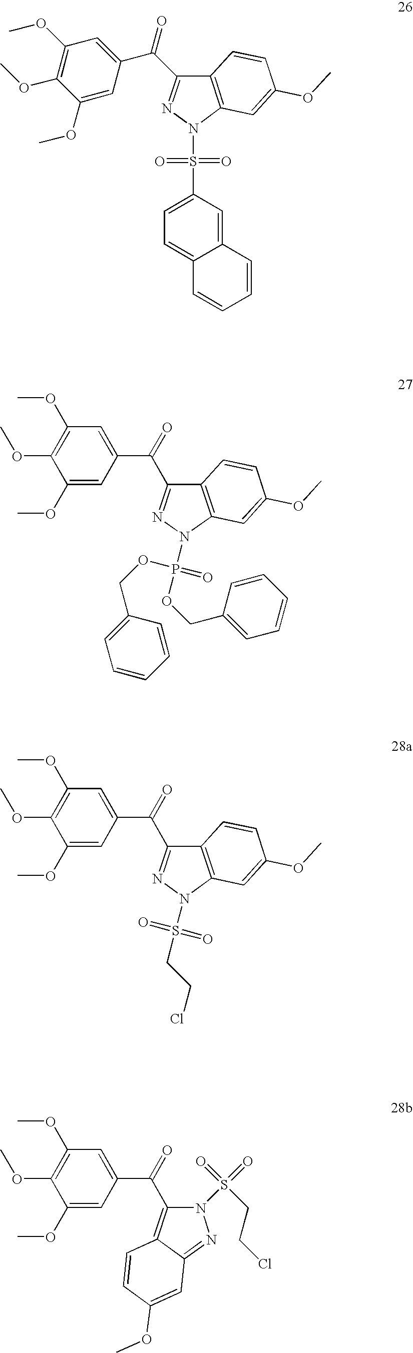

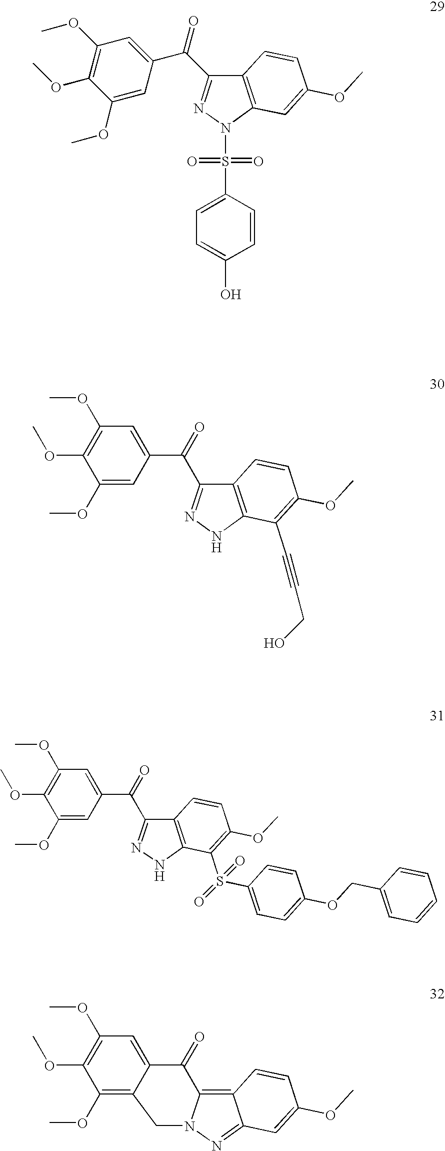

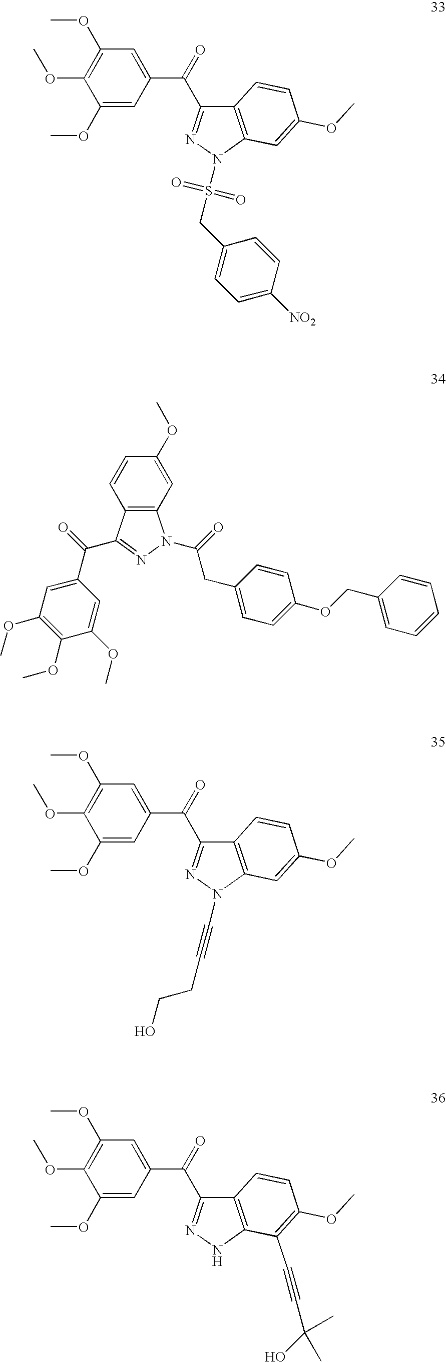

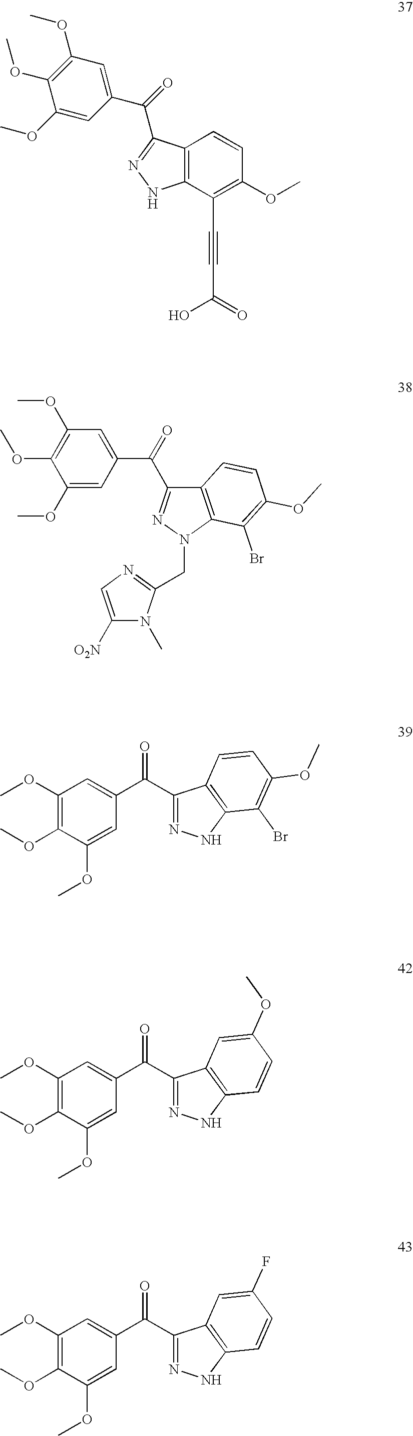

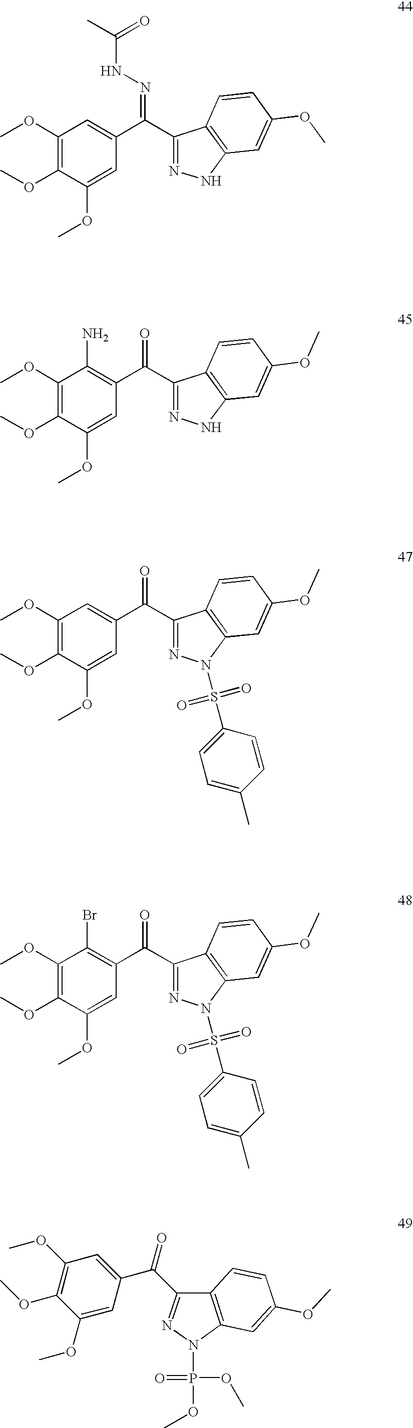

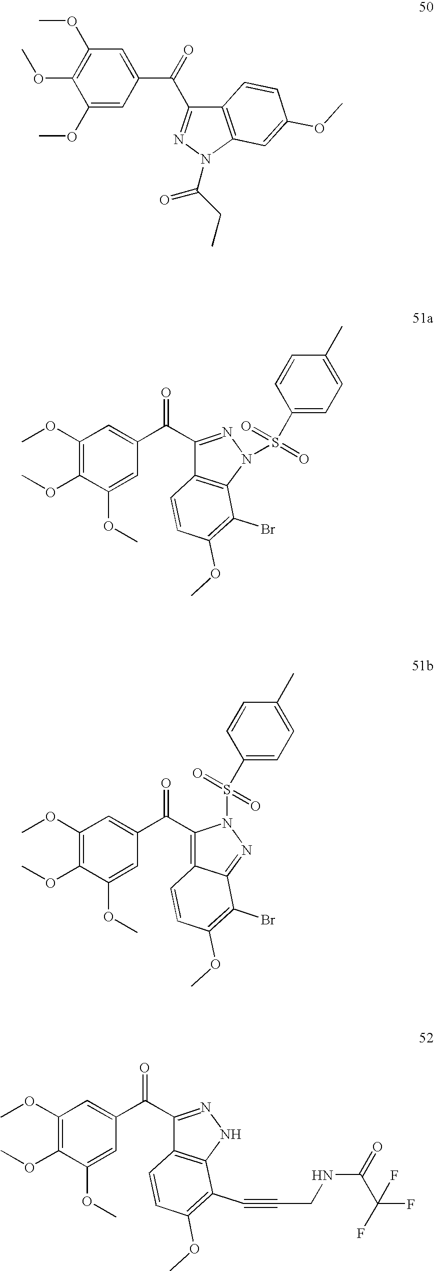

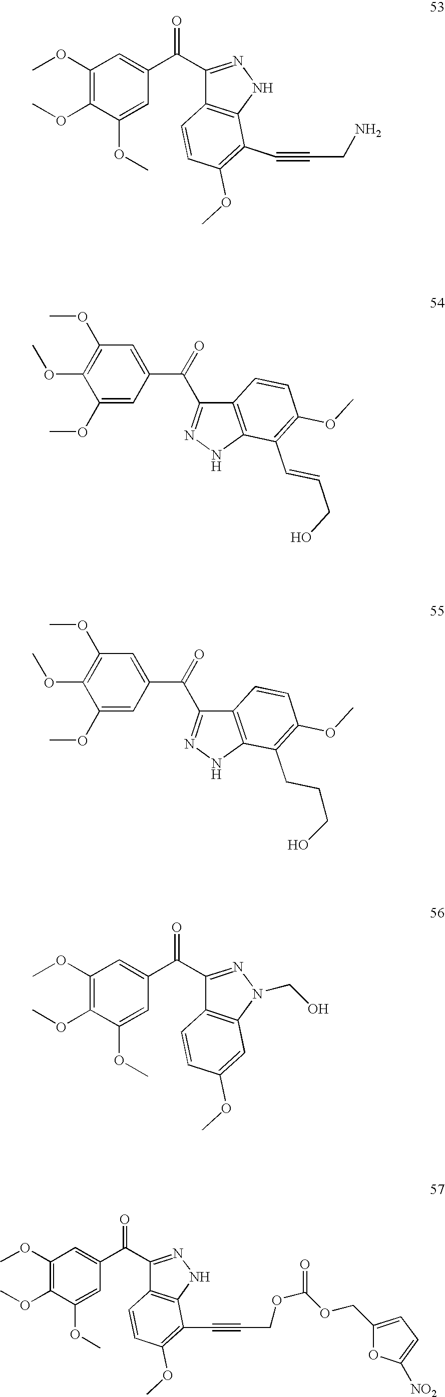

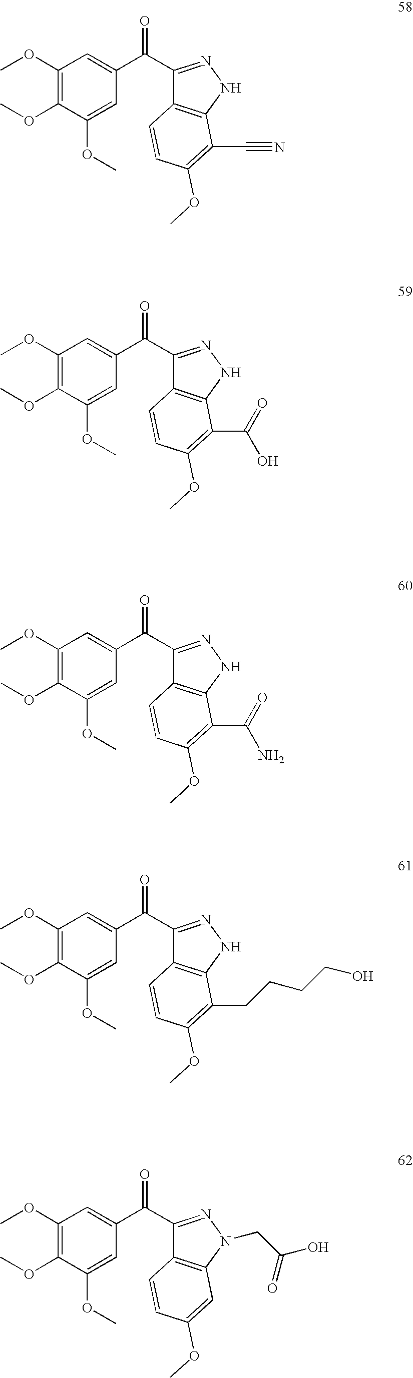

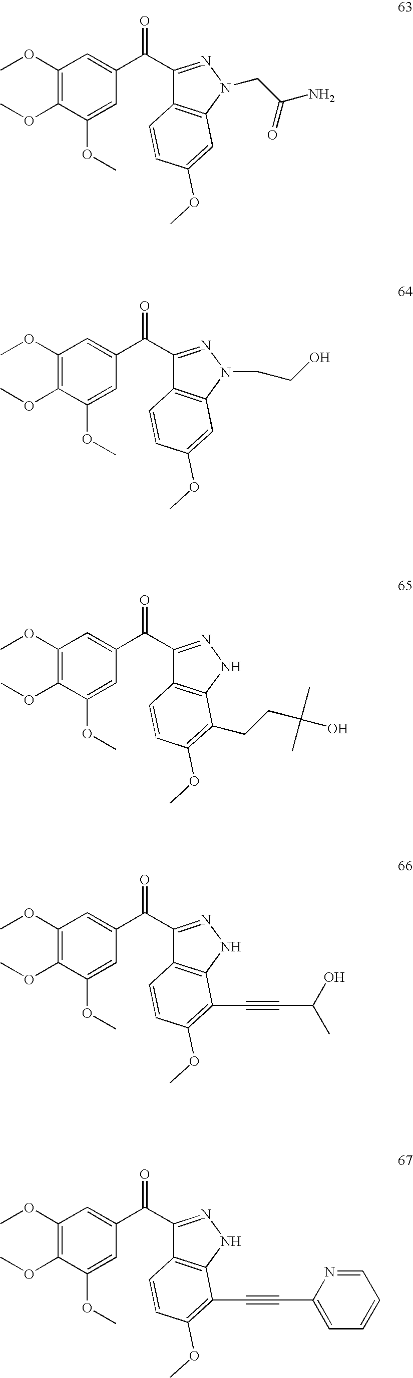

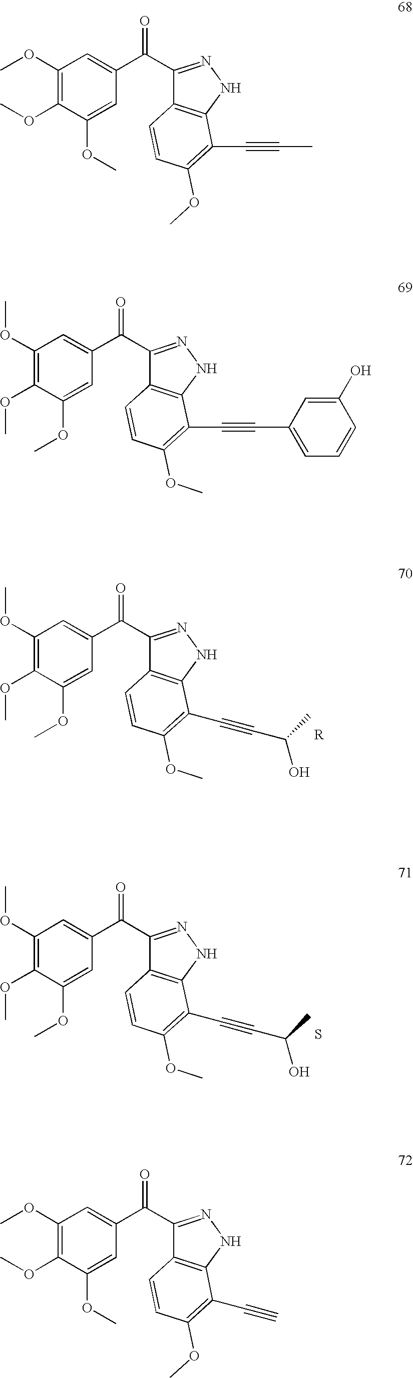

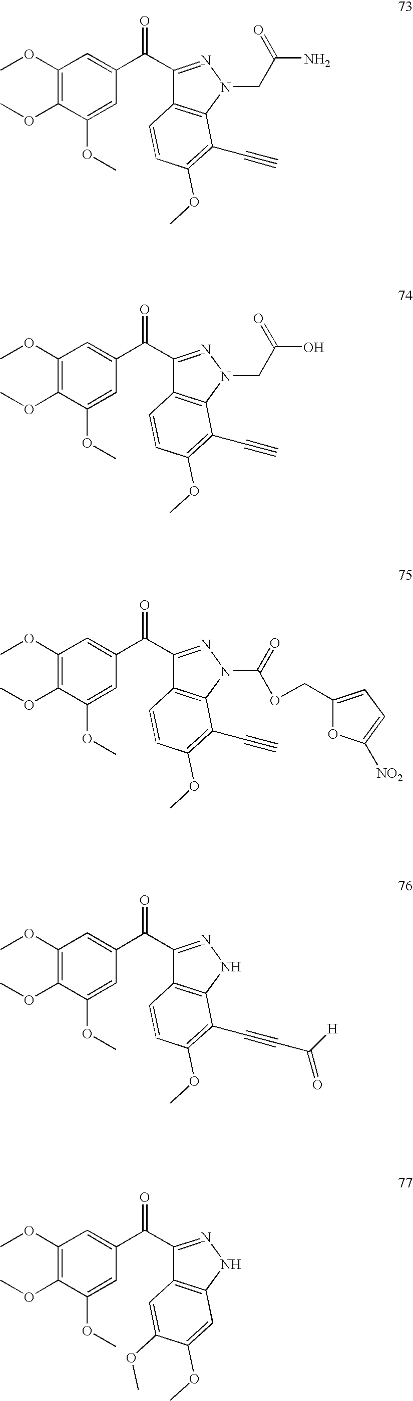

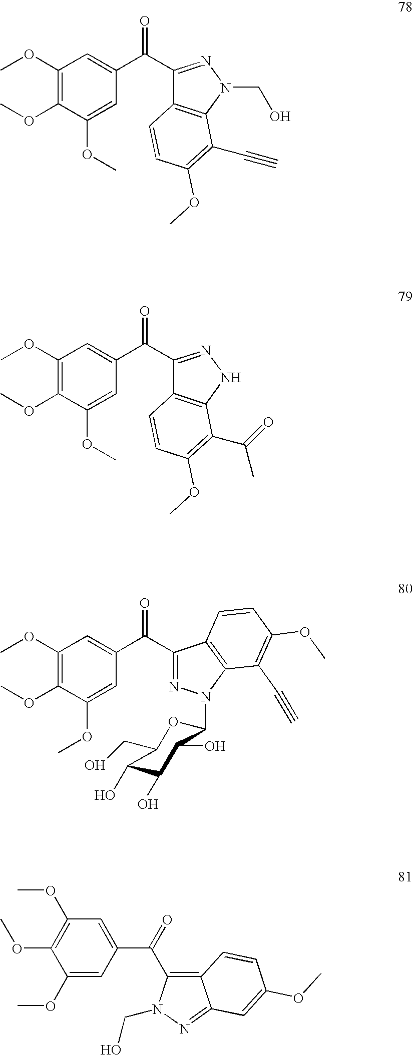

and a tautomer or an individual isomer or a racemic or non-racemic mixture of isomers, a polymorph, a hydrate, a prodrug or a pharmaceutically acceptable salt or solvate thereof.

In one embodiment, compounds 9, 10, 11, 14, 21, 22, 30, 35-45, 52-61, 64-72, and 76-81 are bonded through a hydroxyloxygen or an amine nitrogen to a hypoxic activator (Hyp) to provide a hypoxically activated prodrug having (-OHyp) or (-NHyp).

Functional characteristics of tubulin binding compounds: In one embodiment, the compounds and prodrugs suited for use in the invention are tubulin binding compounds when administered to a human, non-human primate, or other mammal. As is usual in the pharmaceutical arts, not every structural analog of a compound (e.g., a tubulin binding compound) is pharmacologically active. Active forms can be identified by routine screening of the compounds of the invention for the activity. A variety of assays and tests can be used to assess pharmacological activity of a compound or novel prodrug of the invention, including in vitro assays, such as those described below and elsewhere herein, in vivo assays in humans, non-human primates and other mammals, and/or clinical studies.

In some embodiments of the invention in which a tubulin binding compound is used for treatment or prevention of cancer or its manifestations, a tubulin binding compound with similar apoptosis-inducing activity similar to that of Combretastatin A-4 phosphate is selected. Thus, in some embodiments of the invention, a topoisomerase inhibitor that induces apoptosis in cancer cells such as H460, PC3, CCRF, LNCaP, HT29, MESSA and PWR-1E is administered to treat cancer.

In some embodiments of the invention in which a tubulin binding compound is used for treatment or prevention of a hyperproliferative disease or its manifestations, a tubulin binding compound with similar apoptosis-inducing activity similar to that of Combretastatin A-4 phosphate is selected. Thus, in some embodiments of the invention, a tubulin binding compound that induces apoptosis in skin, epithelial or endothelial, nerve, and T cells, is administered to treat a hyperproliferative disease, e.g. psoriasis, rheumatoid arthritis, restenosis, benign prostatic hyperplasia, and multiple sclerosis.

In one aspect, the present invention provides a compound of formula (I-VII) having a GI₅₀, GI₉₀, IC₅₀, or IC₅₀ of about 0.001 to about 1000 nM, about 0.01 to about 100 nm, about 0.1 to about 50 nM, and about 1 to about 10 nM in a cancer cell antiproliferation assay. In one embodiment, the present invention provides a compound of formula (I) having a GI₅₀ or IC₅₀ of about 0.01 to about 100 nm, about 0.1 to about 50 nm, and about 1 to about 10 nm in a cancer cell antiproliferation assay. In various embodiments, said antiproliferation assays employ cancer cell including but not limited to gastric, colon, breast, and non-small cell lung cancer. In various embodiments, the gastric cancer cell used is MESSA or doxorubicin resistant MESSA/DX5 cell; the colon cancer cell is HT29 cell; the breast cancer cell is T47D cell; and the non-small cell lung cancer cell is H460 cell.

In one embodiment, the present invention provides a compound having a GI₅₀ or IC₅₀ of about 1 to about 50 nM in a cancer cell antiproliferation assay, such as, for example, compounds 30, 37, 39, 54, 55, 66, 68, 70, 71, and 72. In one embodiment, the present invention provides a tubulin binding compound having an IC₅₀ of tubulin polymerization of about 0.1 to about 10 μM as determined in a tubulin polymerization inhibition assay, such as for example, compounds 30 and 39.

In one aspect, the present invention provides a compound which when subjected to a liver microsomal stability study, remains about 10 to about 100, about 20 to about 80, about 80 to about 100% unmetabolized. In one embodiment, the liver microsomal study is conducted for between 10-60, 20-40, or 25-35 minutes. In one embodiment, mouse liver microsome is employed in the study. Examples of compounds remaining 80-100% unchanged in a mouse liver microsomal stability study include but are not limited to, compounds 30, 60, 66, and 70.

In one aspect, the present invention provides a compound which when subjected to a plasma stability study, remains about 10-100, 20-80, or 80-100% unmetabolized. In one embodiment, the plasma stability study is conducted for between 10-60, 20-40, or 25-35 minutes. In one embodiment, the plasma employed is from the same species of mammal the liver of whch is employed in the liver microsomal stability study. Examples of compounds remaining 80-100% unchanged in a mouse plasma stability study include but are not limited to, compounds 30, 35, 70, 71, and 72.

In one aspect, the present invention provides a compound which upon administration to a human cancer cell xenograft tumor bearing mice, can reduce the tumor volume to about 5-70% of a control tumor volume. In one embodiment, the compound is of formula (I)-(XLVII). In one embodiment, the compound is of formula (I-VII). In one embodiment, the human cancer cell used is H460 cell. In one embodiment, the compound administered is of formula (I). An example of a compound useful in reducing mice xenograft tumor is compound 30.

In one aspect, the present invention provides a pharmaceutically acceptable formulation of the compounds of the invention, wherein the pharmaceutically acceptable carrier, dilutent, or excipient is selected from a polyethylene glycol (PEG). In one embodiment, the pharmaceutically acceptable formulation comprises a compound of formula (I)-(XLVII). In one embodiment, the pharmaceutically acceptable formulation comprises a compound of formula (I)-(VIII). In one embodiment, the pharmaceutically acceptable formulation comprises a compound of formula (I). Compound 30, for example, can be formulated with a PEG to yield a pharmaceutically acceptable formulation.

### Methods of Synthesis

In one aspect the present invention provides novel methods for the synthesis of the compounds of this invention. The Fedenok et al. Tetrahedron Lett., 2003, 44: 5453-5455, Yokoe et al., Heterocycles 1985, 23 (6):1395-1398, Hachiken et al., J. Heterocyclic Chem., 1988, 25:327-331, Makosza et al., Eur. J. Org. Chem., 2000, 1:193-198, Nefedov et al., Russ. J. Org. Chem., 1994, 30(11): 1724-1728, Hlastav et al., 1998, Heterocycles, 48, 5:1015-1022, Wu et al., J. Fluorine Chem., 2003, 122(2):171-174, and Scholtz et al., Chem. Ber., 1913, 46: 1077 references describe method for the synthesis of various aroyl-heterocycles useful for other purposes. Novel compounds of this invention can be synthesized by adapting these aforementioned procedures. The aroylindazole compounds of this invention can be synthesized by adapting known method to synthesize aroyl indazoles useful for other purposes according to the methods provided by this invention. Prodrug compounds of this invention can be synthesized using the novel compounds of the invention and known tubulin binding as described herein as starting material. Known tubulin binding compounds and methods of their synthesis are described, for example, in the references, Martino et al., J. Med. Chem., 2004, ASAP articles; Mahboobi et al., J. Med. Chem. 2001, 44, 4535-53; Gastper et al. J. Med. Chem., 1998, 49, 4965-72; Bacher et al., Pure Appl. Chem., 2001, 73(9): 1459-64; Lee et al., WO 98/39332; Combeau et al., WO 02/072575; Nam et al.; and Hsieh et al. (supra, each of which is incorporated herein by reference).

In another aspect the present invention provides a method for synthesizing a compound of the present invention comprising the steps of

(i) halogenating the compound of formula

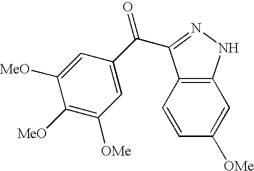

to yield product-1 of formula

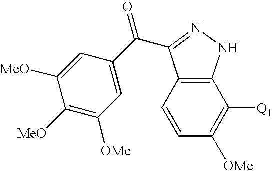

wherein Q₁ is Cl, Br, or I;
(ii) optionally reacting product-1 with H-Q₁ wherein Q₁ is

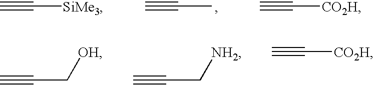

or protected forms thereof to yield product-2 of formula

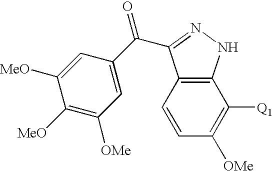

wherein Q1 is

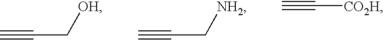

or protected forms thereof, and
(iii) optionally reducing product-2 obtained in step (ii) to yield the compound or prodrug of the invention.

In one embodiment, step (i) is performed by employing N-halosuccinimide. In another embodiment, step (ii) is performed by further employing a Cu(0); a Pd(II); Pd(0) based catalyst. In another embodiment, step (ii) is performed by employing a Sonogashira coupling.

Methods for making compound of the present invention are described below in Schemes 1-5:

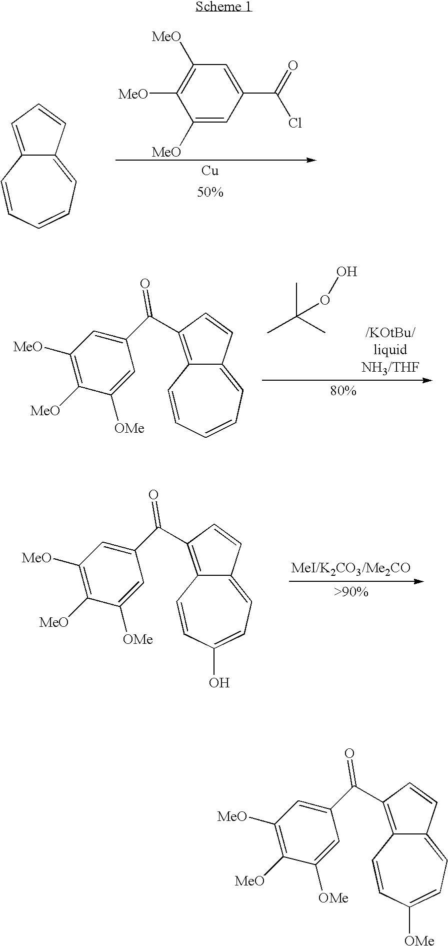

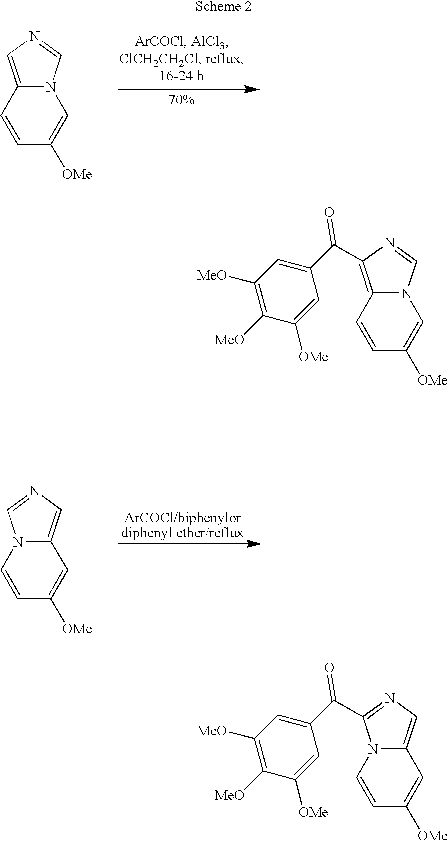

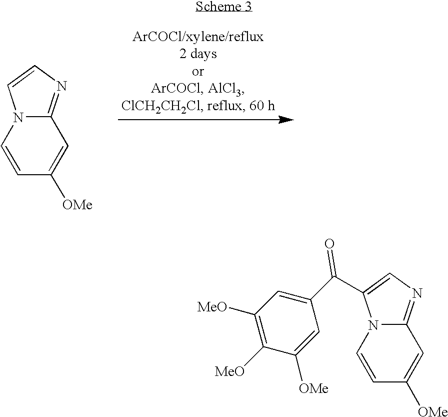

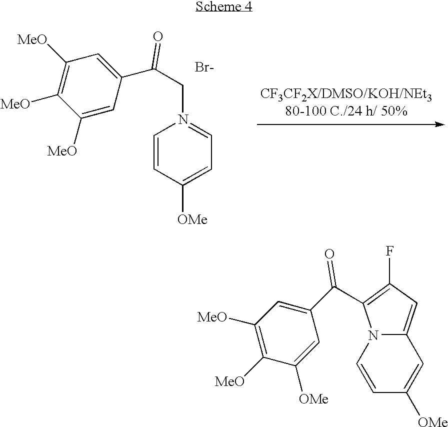

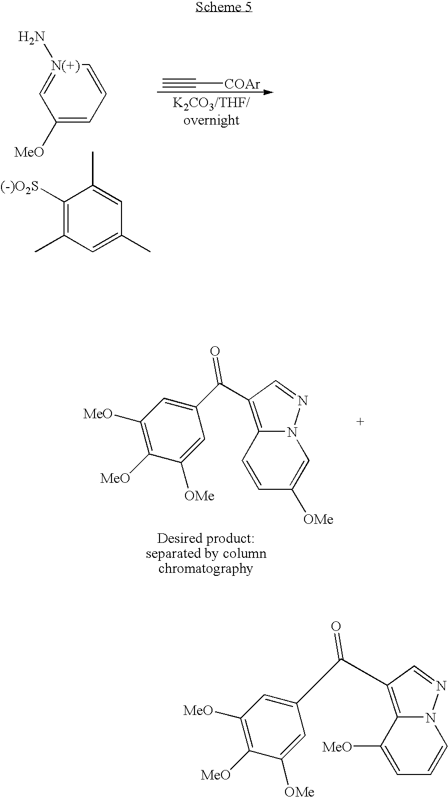

In one embodiment, methods for the synthesis of the compounds of this invention can be identified in accordance with the present invention via search tools such as SciFinder from the American Chemical Society and Beilstein from MDL Software. Illustrative methods for making anti-cancer compounds of the present invention in accordance with this disclosure are provided in the EXAMPLES section below.

### Pharmaceutical Compositions

For use as a prophylactic or therapeutic agent, a compound of the present invention disclosed herein (including pharmaceutically acceptable salts, solvates, hydrates, and prodrugs) is usually formulated as a pharmaceutical composition comprising the compounds or the prodrugs of this invention and a pharmaceutically-acceptable carrier. The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient. Pharmaceutical preparations for oral use can be obtained through combining active compounds with solid excipient and, optionally, other compounds. Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances which increase the viscosity of the suspension, such as sodium carboxymethyl cellulose, sorbitol, or dextran. For topical or nasal administration, penetrants appropriate to the particular barrier to be permeated are used in the formulation. Such penetrants are generally known in the art.

Further details on techniques for formulation and administration can be found in the latest edition of Remington's Pharmaceutical Sciences (Maack Publishing Co., Easton, Pa.); GOODMAN AND GILMAN'S: THE PHARMACOLOGICAL BASIS OF THERAPEUTICS 10^{TH} EDITION 2001 by Louis Sanford Goodman et al., McGraw-Hill Professional; PHARMACEUTICAL DOSAGE FORMS AND DRUG DELIVERY SYSTEMS 7^{th} Edition Howard C. Ansel, et al., 2004, Lippincott Williams & Wilkins Publishers; PHARMACEUTICAL CALCULATIONS 11^{th} Edition, 2001, by Mitchell J. Stoklosa et al., Lippincott Williams & Wilkins; PHYSICAL PHARMACY: PHYSICAL CHEMICAL PRINCIPLES IN THE PHARMACEUTICAL SCIENCES 4^{th} Edition by Pilar Bustamante, et al., 1993, Lea & Febiger.

### Dosages and Administration

A variety of routes, dosage schedules, and dosage forms are appropriate for administration of pharmaceutical compositions of the invention. Appropriate dosage schedules and modes of administration will be apparent to the ordinarily skilled practitioner upon reading the present disclosure and/or can be determined using routine pharmacological methods and/or methods described herein.

The dose, schedule and duration of administration of the compound and/or prodrug of the invention will depend on a variety of factors. The primary factor, of course, is the choice of a specific compound or prodrug of the present invention. Other important factors include the age, weight and health of the subject, the severity of symptoms, if any, the subject's medical history, co-treatments, goal (e.g., prophylaxis or prevention of relapse), preferred mode of administration of the drug, the formulation used, patient response to the drug, and the like.

For example, a compound and/or a prodrug of the invention can be administered at a dose in the range of about 0.1 mg to about 500 mg of a compound and/or prodrug of the invention per kg of body weight of the patient to be treated per day, optionally with more than one dosage unit being administered per day, and typically with the daily dose being administered on multiple consecutive days. In one embodiment, the compounds of the present invention include novel compounds of the invention, novel prodrug thereof, and novel prodrugs of known compounds. In one embodiment, a compound and/or a prodrug of the invention is administered in a daily dose in the range of about 0.5 mg to about 400 mg/Kg; about 1.0 mg to about 300 mg/Kg; about 1.5 mg to about 250 mg/Kg; about 2.0 mg to about 200 mg/Kg; about 2.5 mg to about 150 mg/Kg; about 5 to about 100 mg/Kg; about 10 to about 50 mg/Kg; and about 10 to about 70 mg per kg of body weight of the patient to be treated.

Cell culture studies are frequently used in the art to optimize dosages, and the assays disclosed herein can be used in determining such doses.

For illustration, a therapeutically or prophylactically effective dose of a compound and/or a prodrug of the invention can be administered daily or once every other day or once a week to the patient. Controlled and sustained release formulations of the analogs can be used. Generally, multiple administrations of the compound and/or prodrug of the invention are employed. For optimum treatment benefit, the administration of the prophylactically effective dose can be continued for multiple days, such as for at least five consecutive days, and often for at least a week and often for several weeks or more. In one embodiment, the compound and/or prodrug of the invention is administered once (qday), twice (bid), three times (tid), or four times (qid) a day or once every other day (qod) or once a week (qweek), and treatment is continued for a period ranging from three days to two weeks or longer.

In one aspect, the present invention provides a method for treating cancer or other hyperproliferative diseases by administering to a patient in need of therapy thereof a therapeutically effective dose of a compound or prodrug compound of the invention. In one embodiment, the present invention provides a method for treating cancer or other hyperproliferative diseases by administering about 0.1 to about 500 mg/Kg of a compound or a prodrug compound of the invention to a patient in need of therapy thereof. In one embodiment, a compound and/or a prodrug of the invention is administered in a daily dose in the range of about 0.5 mg to about 400 mg/Kg; about 1.0 mg to about 300 mg/Kg; about 1.5 mg to about 250 mg/Kg; about 2.0 mg to about 200 mg/Kg; about 2.5 mg to about 150 mg/Kg; about 5 to about 100 mg/Kg; about 10 to about 50 mg/Kg; and about 10 to about 70 mg per kg of body weight of the patient to be treated. In one embodiment, the present invention provides a unit dosage form of about 1 to about 200 mg of a compound or prodrug compound of the invention to a patient in need of therapy thereof.

Additional guidance concerning administration of the compounds of the present invention may be obtained from such information known for other tubulin binding compounds. For example, Combretastatin A-4 phosphate (CA4P), a tubulin-binding compound is reported to have a maximum tolerated daily dose of 60-68 mg/m², and has, for example, been administered to patients in clinical trials in daily doses of 27 and 36 mg/m², by a 10-minute infusion, once every 21 days (Young et al., 2004, Expert Opin. Investig. Drugs, 13(9):1171-82 and Bilenker et al., 2005, Clin. Cancer Res., 11(4):1527-33). The compounds of the present invention can be administered in similar daily doses for treatment of cancer. Therefore, in one embodiment, a compound of the present invention can be administered in a therapeutically affective daily dose of about 10 to about 100 mg/m², about 20 to about 80 mg/m², about 30 to about 70 mg/m², about 40 to about 60 mg/m², and about 45 to about 55 mg/m² to treat cancer. A dose in mg/m² can be converted to a mg/kg dose in adult humans by dividing the mg/m² dose by a factor of 37; in children the corresponding dividing factor is 25. In one embodiment, a compound of the present invention can be administered in a therapeutically affective daily dose of about 0.3 to about 3 mg/kg, about 0.6 to about 2.4 mg/kg, about 0.9 to about 2.1 mg/kg, about 1.2 to about 1.8 mg/kg, and about 1.4 to about 1.6 mg/kg to treat cancer.

Of course modern cancer therapy often involves administering of a drug “cocktail” in which several anti-cancer drugs are contemporaneously administered to a cancer patient. The novel compounds of the present invention and the prodrug compounds of the invention can be used in such therapies either in addition to or in substitution of one or more of the co-administered drugs. Also, because there may be cancer cells in a patient that are normoxic and located adjacent to a hypoxic region of a tumor, one can, in one embodiment of the invention, co-administering a prodrug of the invention with one or more other drugs that target normoxic cells.

In one embodiment, the hyperproliferative disease is selected from the group consisting of angiofibroma, atherosclerosis, benign prostatic hyperplasia, corneal graft rejection, gout, graft versus host disease, glaucoma, inflammatory diseases such as inflammatory bowel disease, ischemic heart and peripheral vascular disease, Karposi's sarcoma, keloids, life threatening infantile hemangiomas, macular degeration, myocardial angiogenesis, myocardial infraction, multiple sclerosis, neovascular-based dermatological conditions, Osler-Webber Syndrome, osteoarthritis, psoriasis, psoriatic arthritis, pulmonary fibrosis, psoriasis, rheumatoid arthritis, restenosis, rheumatoid arthritis, scleroderma, telangectasia, and wound granularization.

### Combination Therapies

In one embodiment, a compound and/or a prodrug compound of the invention can be co-administered in combination with other anti-cancer agents (“anticancer agent”). Without intending to be bound by any particular mechanism or effect, such co-administration can in some cases provide one or more of several advantages over known cancer therapies, such as, for example co-administration of a compound and/or a prodrug compound of the invention and the anticancer agent has a synergistic effect on induction of cancer cell death. Co-administration provides a better therapeutic result than administration of the anticancer agent alone, e.g., greater alleviation or amelioration of one or more symptoms of the cancer, diminishment of extent of disease, delay or slowing of disease progression, amelioration, palliation or stabilization of the disease state, partial or complete remission, prolonged survival or other beneficial therapeutic results.

The co-administration of a compound and/or a prodrug compound compound of the invention increases the sensitivity of cancer cells to the anticancer agent, allowing lower doses of the anticancer agent to be administered to the patient or allowing an anticancer agent to be used for treatment of cells otherwise resistant to the anticancer agent or otherwise refractory to treatment. Generally anti-cancer agents target rapidly dividing cells in the normoxic region, the prodrug compounds of the invention target the hypoxic cells in the regions of tumors that are not efficiently killed by the anticancer agent alone.

As used herein, a compound and/or a prodrug compound of the invention is “co-administered” with another anticancer agent (also referred to herein as, “Agent”) wherein a compound and/or a prodrug compound of the invention and Agent are administered as part of the same course of therapy. In one embodiment, a compound and/or a prodrug compound of the invention is first administered prior to administration of the Agent, (i.e., the initiation of the other cancer therapy), and treatment with the compound and/or prodrug compound of the invention is continued throughout the course of administration of the Agent (i.e., the course of the other therapy). In another embodiment, a compound and/or a prodrug compound of the invention is administered after the initiation or completion of the other cancer therapy. In other embodiments, a compound and/or a prodrug compound of the invention is first administered contemporaneously with the initiation of the other cancer therapy.

In one embodiment, a compound and/or a prodrug compound of the invention is first administered prior to administration of the Agent, and treatment with the compound and/or prodrug compound of the invention is continued after the cessation of administration of the Agent. In one embodiment, a compound and/or a prodrug compound of the invention is first administered prior to administration of the Agent, and treatment with the compound and/or prodrug compound of the invention is continued during part of the period of administration of the Agent. For certain drugs, such as certain topoisomerase inhibitors, administration of a compound and/or a prodrug compound of the invention can be initiated and completed prior to the administration of the second drug.

In the presence of oxygen, the radical anion formed upon the reduction of Hyp reacts with oxygen to yield superoxide and Hyp. Superoxide is a cytotoxin and the production of superoxide in normoxic tissues can lead to unwanted side effects. In one embodiment, the present invention provides a method wherein a compound and/or a prodrug compound of the invention administered in combination with a chemoprotective agent or a chemoprotectant. Chemoprotective agents protect healthy tissue from the toxic effects of anticancer drugs. In one embodiment, the chemoprotective agent is a thiol or a disulfide. In one embodiment, the chemoprotectant can reduce superoxide. In another embodiment, the chemoprotectant can react with the “Michael-receptor” generated from a hypoxia activated prodrug of the invention and prevent “Michael-receptor” from reacting with proteins and nucleic acid.

Anticancer drug therapy today typically involves multiple rounds, or “cycles,” of administration of the anti-cancer agent(s). In the context of administering a compound and/or a prodrug compound of the invention, each cycle of administration (as well as a complete set of cycles) can be viewed as administration of a second drug. A compound and/or a prodrug compound of the invention can be administered in any or all of the multiple cycles of treatment with the other Agent; in general, the compound and/or prodrug compound of the invention is administered on a daily basis for at least two or more days during each cycle. In one aspect of the invention, a compound and/or a prodrug compound of the invention is co-administered with the Agent according to a schedule repeated at each round.

In one version of the method of treating cancer using the a compound and/or a prodrug compound of the invention, the compound and/or prodrug compound of the invention is administered in combination with an effective amount of one or more chemotherapeutic agents, an effective amount of radiotherapy, an appropriate surgery procedure, or any combination of such additional therapies.

When a compound and/or a prodrug compound of the invention is used in combination with one or more of the additional therapies, the compound and/or prodrug compound of the invention and additional therapy can be administered at the same time or can be administered separately. For example, if a compound and/or a prodrug compound of the invention is administered with an additional chemotherapeutic agent, the two agents can be administered simultaneously or can be administered sequentially with some time between administrations. One of skill in the art will understand methods of administering the agents simultaneously and sequentially and possible time periods between administrations.

The Agents can be administered as the same or different formulations and can be administered via the same or different routes.

Chemotherapeutic agents that can be used in combination with the compound of the invention include, but are not limited to, busulfan, improsulfan, piposulfan, benzodepa, carboquone, 2-deoxy-D-glucose, lonidamine and analogs thereof (refrence apps), glufosfamide, meturedepa, uredepa, altretamine, imatinib, triethylenemelamine, triethylenephosphoramide, triethylenethiophosphoramide, trimethylolomelamine, chlorambucil, chlornaphazine, estramustine, ifosfamide, gefitinib, mechlorethamine, mechlorethamine oxide hydrochloride, melphalan, novembichin, phenesterine, prednimustine, trofosfamide, uracil mustard, carmustine, chlorozotocin, fotemustine, nimustine, ranimustine, dacarbazine, mannomustine, mitobronitol, mitolactol, pipobroman, aclacinomycins, actinomycin F(1), anthramycin, azaserine, bleomycin, cactinomycin, carubicin, carzinophilin, chromomycin, dactinomycin, daunorubicin, daunomycin, 6-diazo-5-oxo-1-norleucine, mycophenolic acid, nogalamycin, olivomycin, peplomycin, plicamycin, porfiromycin, puromycin, streptonigrin, streptozocin, tubercidin, ubenimex, zinostatin, zorubicin, denopterin, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, 5-fluorouracil, tegafur, L-asparaginase, pulmozyme, aceglatone, aldophosphamide glycoside, aminolevulinic acid, amsacrine, bestrabucil, bisantrene, carboplatin, defofamide, demecolcine, diaziquone, elformithine, elliptinium acetate, etoglucid, flutamide, gallium nitrate, hydroxyurea, interferon-alpha, interferon-beta, interferon-gamma, interleukin-2, lentinan, mitoguazone, mitoxantrone, mopidamol, nitracrine, pentostatin, phenamet, pirarubicin, podophyllinic acid, 2-ethylhydrazide, procarbazine, razoxane, sizofuran, spirogermanium, paclitaxel, tamoxifen, erlotonib, teniposide, tenuazonic acid, triaziquone, 2,2′,2″-trichlorotriethylamine, urethan, vinblastine, cyclophosphamide, and vincristine. Other chemotherapeutic agents that can be used include platinum derivatives, including but not limited to cis platinum, carboplatin, and oxoplatin.

In one version, a compound and/or a prodrug compound of the invention can be used in combination with an angiogenesis inhibitor including but not limited to Avastin and similar therapeutics. In one version of the combination treatment methods, a subject is treated with an angiogenisis inhibitor and subsequently treated with a compound and/or a prodrug compound of the invention. In one version of these combination methods of treatment using an angiogenesis inhibitor, the method is used to treat breast cancer.

In another embodiment, a compound and/or a prodrug compound of the invention is administered with an anti-cancer agent that acts, either directly or indirectly, to inhibit the epidermal growth factor or EGFR receptor. EGFR inhibitors suitable for coadministration with a compound of the invention include gefitinib and erlotonib.

In another version, a compound and/or a prodrug compound of the invention is administered with an anti-cancer agent that acts, either directly or indirectly, to inhibit hypoxia-inducible factor 1 alpha (HIF1a) or to inhibit a protein or enzyme, such as a glucose transporter or VEGF, whose expression or activity is increased upon increased HIF1a levels. HIF1a inhibitors suitable for use in this version of the methods and compositions described herein include P13 kinase inhibitors; LY294002; rapamycin; histone deacetylase inhibitors such as [(E)-(1S,4S,10S,21R)-7-[(Z)-ethylidene]-4,21-diisopropyl-2-oxa-12,13-dithia-5,8,20,23-tetraazabicyclo-[8,7,6]-tricos-16-ene-3,6,9,19,22-pentanone (FR901228, depsipeptide); heat shock protein 90 (Hsp9) inhibitors such as geldanamycin, 17-allylamino-geldanamycin (17-MG), and other geldanamycin analogs, and radicicol and radicicol derivatives such as KF58333; genistein; indanone; staurosporin; protein kinase-1 (MEK-1) inhibitors such as PD98059 (2′-amino-3′-methoxyflavone); PX-12 (1-methylpropyl 2-imidazolyl disulfide); pleurotin PX-478; quinoxaline 1,4-dioxides; sodium butyrate (NaB); sodium nitropurruside (SNP) and other NO donors; microtubule inhibitors such as novobiocin, panzem (2-methoxyestradiol or 2-ME2), vincristines, taxanes, epothilones, discodermolide, and derivatives of any of the foregoing; coumarins; barbituric and thiobarbituric acid analogs; camptothecins; and YC-1, a compound described in Biochem. Pharmacol., 15 Apr. 2001, 61(8):947-954, incorporated herein by reference, and its derivatives.

In another version, a compound and/or a prodrug compound of the invention is administered with an anti-angiogenic agent, including but not limited to anti-angiogenic agents selected from the group consisting of angiostatin, an agent that inhibits or otherwise antagonizes the action of VEGF, batimastat, captopril, cartilage derived inhibitor, genistein, endostatin, interleukin, lavendustin A, medroxypregesterone acetate, recombinant human platelet factor 4, Taxol, tecogalan, thalidomide, thrombospondin, TNP-470, and Avastin. Other useful angiogenesis inhibitors for purposes of the combination therapies provided by the present methods and compositions described herein include Cox-2 inhibitors like celecoxib (Celebrex), diclofenac (Voltaren), etodolac (Lodine), fenoprofen (Nalfon), indomethacin (Indocin), ketoprofen (Orudis, Oruvail), ketoralac (Toradol), oxaprozin (Daypro), nabumetone (Relafen), sulindac (Clinoril), tolmetin (Tolectin), rofecoxib (Vioxx), ibuprofen (Advil), naproxen (Aleve, Naprosyn), aspirin, and acetaminophen (Tylenol).

In addition, because pyruvic acid plays an important role in angiogenesis, pyruvate mimics and glycolytic inhibitors like halopyruvates, including bromopyruvate, can be used in combination with an anti-angiogenic compound and a compound and/or a prodrug compound of the invention to treat cancer. In another version, a compound and/or a prodrug compound of the invention is administered with an anti-angiogenic agent and another anti-cancer agent, including but not limited to a cytotoxic agent selected from the group consisting of alkylators, Cisplatin, Carboplatin, and inhibitors of microtubule assembly, to treat cancer.

In addition to the combination of a compound and/or a prodrug compound of the invention with the Agents described above, the present methods and compositions described herein provides a variety of synergistic combinations of the compound and/or prodrug compound of the invention and other anti-cancer drugs. Those of skill in the art can readily determine the anti-cancer drugs that act “synergistically” with a compound and/or a prodrug compound of the invention as described herein. For example, the reference Vendetti, “Relevance of Transplantable Animal-Tumor Systems to the Selection of New Agents for Clinical Trial,” Pharmacological Basis of Cancer Chemotherapy, Williams and Wilkins, Baltimore, 1975, and Simpson Herren et al., 1985, “Evaluation of 1n Vivo Tumor Models for Predicting Clinical Activity for Anticancer Drugs,” Proc. Am. Assoc. Cancer Res. 26: 330, each of which is incorporated herein by reference, describe methods to aid in the determination of whether two drugs act synergistically.

While synergy is not required for therapeutic benefit in accordance with the methods of described herein, in one embodiment, the present invention provides a method of cancer treatment, wherein there is synergy between a compound and/or a prodrug compound of the invention and another anticancer agent. Two drugs can be said to possess therapeutic synergy if a combination dose regimen of the two drugs produces a significantly better tumor cell kill than the sum of the single Agents at optimal or maximum tolerated doses. The “degree of synergy” can be defined as net log of tumor cell kill by the optimum combination regimen minus net log of tumor cell kill by the optimal dose of the most active single Agent. Differences in cell kill of greater than ten-fold (one log) are considered conclusively indicative of therapeutic synergy.

When a compound and/or a prodrug compound of the invention is used with another anti-cancer agent, the compound and/or prodrug compound of the invention will, at least in some versions, be administered prior to the initiation of therapy with the other drug or drugs and administration will typically be continued throughout the course of treatment with the other drug or drugs. In some versions, the drug co-administered with a compound and/or a prodrug compound of the invention will be delivered at a lower dose, and optionally for longer periods, than would be the case in the absence of administering the compound and/or prodrug of the invention. Such “low dose” therapies can involve, for example, administering an anti-cancer drug, including but not limited to paclitaxel, docetaxel, doxorubicin, cisplatin, or carboplatin, at a lower than approved dose and for a longer period of time together with a compound and/or a prodrug compound of the invention administered in accordance with the methods described herein.

These methods can be used to improve patient outcomes over currently practiced therapies by more effectively killing cancer cells or stopping cancer cell growth as well as diminishing unwanted side effects of the other therapy. In other versions, the other anti-cancer agent or agents will be administered at the same dose levels used when a compound and/or a prodrug compound of the invention is not co-administered. When employed in combination with a compound and/or a prodrug compound of the invention, the additional anti-cancer agent(s) is dosed using either the standard dosages employed for those Agents when used without the compound and/or prodrug compound of the invention or are less than those standard dosages.

The administration of a compound and/or a prodrug compound of the invention in accordance with the methods described herein can therefore allow the physician to treat cancer with existing (or later approved) drugs at lower doses (than currently used), thus ameliorating some or all of the toxic side effects of such drugs. The exact dosage for a given patient varies from patient to patient, depending on a number of factors including the drug combination employed, the particular disease being treated, and the condition and prior history of the patient, but can be determined using only the skill of the ordinarily skilled artisan in view of the teachings herein.

Specific dose regimens for known and approved chemotherapeutic agents or antineoplastic agents (i.e., the recommended effective dose) are known to physicians and are given, for example, in the product descriptions found in the Physician's Desk Reference 2003, (Physicians' Desk Reference, 57th Ed) Medical Economics Company, Inc., Oradell, N.J. and/or are available from the Federal Drug Administration. Illustrative dosage regimens for certain anti-cancer drugs are also provided below.

Cancer drugs can be classified generally as alkylators, anthracyclines, antibiotics, aromatase inhibitors, bisphosphonates, cyclo-oxygenase inhibitors, estrogen receptor modulators, folate antagonists, inorganic aresenates, microtubule inhibitors, modifiers, nitrosoureas, nucleoside analogs, osteoclast inhibitors, platinum containing compounds, retinoids, topoisomerase 1 inhibitors, topoisomerase 2 inhibitors, and tyrosine kinase inhibitors. In accordance with the methods described herein, a compound and/or a prodrug compound of the invention can be co-administered with any anti-cancer drug from any of these classes or can be administered prior to or after treatment with any such drug or combination of such drugs. In addition, a compound and/or a prodrug compound of the invention can be administered in combination with a biologic therapy (e.g., treatment with interferons, interleukins, colony stimulating factors and monoclonal antibodies). Biologics used for treatment of cancer are known in the art and include, for example, trastuzumab (Herceptin), tositumomab and ¹³¹I Tositumomab (Bexxar), rituximab (Rituxan).

Alkylators useful in the practice of the methods described herein include but are not limited to busulfan (Myleran, Busulfex), chlorambucil (Leukeran), ifosfamide (with or without MES NA), cyclophosphamide (Cytoxan, Neosar), glufosfamide, melphalan, L-PAM (Alkeran), dacarbazine (DTIC-Dome), and temozolamide (Temodar). In accordance with the methods described herein a compound and/or a prodrug compound of the invention is co-administered with an alkylator to treat cancer. In one version, the cancer is chronic myelogenous leukemia, multiple myeloma, or anaplastic astrocytoma.

In one embodiment, the present invention provides a method of treating cancer treatable by administering a compound and/or a prodrug compound of the invention alone or in combination with at least another alkylator or a prodrug thereof. Alkylators, such as, for example, cyclophosphamide, ifosfamide, glufosfamide, mechlorethamine, melphalan, chlorambucil, dacarbazine, temozolomide, carmustine, streptozocin, bendamustin, busulfan, thiotepa, cisplatin, carboplatin, and oxaliplatin, and types of cancers treated using any one of such alkylators alone or in combination with other anti cancer or chemoprotective agents are described for example in the reference Hardman et al., (see Hardman et al., The Pharmacological Basis of Therapeutics, 2001, 1389-1399, McGraw-Hill, New York, USA).

In one embodiment, the present invention provides a method of treating cancer by administering a compound and/or a prodrug compound of the invention with a cancer treatment regimen using at least the alkylator Glufosfamide. Glufosfamide is in the clinic for the treatment of pancreatic cancer or Gemzar resistant pancreatic cancer. Glufosfamide can be used for treating breast cancer, Morbus Hodgkin, gastrointestinal tract cancer, or as part of the GCE (Glufosfamide, Carboplatin, and Etoposide) or RGCE (Rituxan and GCE) regimen, for treating lymphomas. (Tidmarsh et al., U.S. Pat. Appl. Nos. 60/638,995, 60/680,451 and 60/719,787). Additional examples of Agents include Terciva, Iressa, Cytarabine and Erbitux.

In one embodiment, the present invention provides a method of treating cancer by administering a compound and/or a prodrug compound of the invention with a cancer treatment regimen using at least a platinum coordination complex alkylator. In one embodiment, the platinum coordination complex alkylator is Cisplatin. Cisplatin can be used to treat cancer of bladder, head and neck, endometrium, small cell carcinoma of the lung, and some neoplasms of childhood. Cisplatin alone or with cyclophosphamide is used to treat advanced ovarian cancer. Combination chemotherapy of Cisplatin with Bleomycin, Etoposide, and Vinblastine is used to treat advanced testicular cancer; and with one of Paclitaxel, Cyclophosphamide, or Doxorubicin to treat ovarian carcinoma.

Anthracyclines useful in the practice of the methods described herein include but are not limited to, doxorubicin (Adriamycin, Doxil, Rubex), mitoxantrone (Novantrone), idarubicin (Idamycin), vairubicin (Valstar), and epirubicin (Ellence). In accordance with the methods described herein a compound and/or a prodrug compound of the invention is co-administered with an anthracycline to treat cancer. In one version, the cancer is acute nonlymphocytic leukemia, Kaposi's sarcoma, prostate cancer, bladder cancer, metastatic carcinoma of the ovary, and breast cancer.

As one example the compound (8S,10S)-10-[(3-Amino-2,3,6-trideoxy-alpha.-L-lyxo-hexopyranosyl)oxy]-8-glycoloyl-7,8,9,10-tetrahydro-6,8,11-trihydroxy-1-methoxy-5,12-naphthacenedione, more commonly known as doxorubicin, is a cytotoxic anthracycline antibiotic isolated from cultures of Streptomyces peucetius var. caesius. Doxorubicin has been used successfully to produce regression in disseminated neoplastic conditions such as acute lymphoblastic leukemia, acute myeloblastic leukemia, Wilm's tumor, neuroblastoma, soft tissue and bone sarcomas, breast carcinoma, ovarian carcinoma, transitional cell bladder carcinoma, thyroid carcinoma, lymphomas of both Hodgkin and non-Hodgkin types, bronchogenic carcinoma, and gastric carcinoma. Doxorubicin is typically administered in a dose in the range of 30-75 mg/m² as a single intravenous injection administered at 21-day intervals; weekly intravenous injection at doses of 20 mg/m²; or 30 mg/m² doses on each of three successive days repeated every four weeks. In accordance with the methods of the methods described herein, a compound and/or a prodrug compound of the invention is co-administered starting prior to and continuing after the administration of doxorubicin at such doses (or at lower doses). Cyclic Anthracycline cytotoxin prodrugs useful in the practice of the methods described herein are provided by the reference Matteuci et al., PCT Patent Aplication No. US05/08161.

Antibiotics useful in the practice of the methods described herein include but are not limited to dactinomycin, actinomycin D (Cosmegen), bleomycin (Blenoxane), daunorubicin, and daunomycin (Cerubidine, DanuoXome). In accordance with the methods described herein a compound and/or a prodrug compound of the invention is co-administered with an antibiotic to treat cancer. In one version, the cancer is a cancer selected from the group consisting of acute lymphocytic leukemia, other leukemias, and Kaposi's sarcoma.

Aromatase inhibitors useful in the practice of the methods described herein include but are not limited to anastrozole (Arimidex) and letroazole (Femara). In accordance with the methods described herein a compound and/or a prodrug compound of the invention is co-administered with an aromatase inhibitor to treat cancer. In one version, the cancer is breast cancer.

Bisphosphonate inhibitors useful in the practice of the methods described herein include but are not limited to zoledronate (Zometa). In accordance with the methods described herein a compound and/or a prodrug compound of the invention is co-administered with a biphosphonate inhibitor to treat cancer. In one version, the cancer is a cancer selected from the group consisting of multiple myeloma, bone metastases from solid tumors, or prostate cancer.

Cyclo-oxygenase inhibitors useful in the practice of the methods described herein include but are not limited to celecoxib (Celebrex). In accordance with the methods described herein a compound and/or a prodrug compound of the invention is co-administered with a cyclo-oxygenase inhibitor to treat cancer. In one version, the cancer is colon cancer or a pre-cancerous condition known as familial adenomatous polyposis.

Estrogen receptor modulators useful in the practice of the methods described herein include but are not limited to tamoxifen (Nolvadex) and fulvestrant (Faslodex). In accordance with the methods described herein a compound and/or a prodrug compound of the invention is co-administered with an estrogen receptor modulator to treat cancer. In one version, the cancer is breast cancer or the treatment is administered to prevent the occurrence or reoccurrence of breast cancer.

Folate antagonists useful in the practice of the methods described herein include but are not limited to methotrexate and tremetrexate. In accordance with the methods described herein a compound and/or a prodrug compound of the invention is co-administered with a folate antagonist to treat cancer. In one version, the cancer is osteosarcoma.

As one example, the compound N-[4-[[(2,4-diamino-6-pteridinyl)methyl methylamino]benzoyl]-L-glutamic acid, commonly known as methotrexate, is an antifolate drug that has been used in the treatment of gestational choriocarcinoma and in the treatment of patients with chorioadenoma destruens and hydatiform mole. It is also useful in the treatment of advanced stages of malignant lymphoma and in the treatment of advanced cases of mycosis fungoides. Methotrexate is administered as follows. For choriocarcinoma, intramuscular injections of doses of 15 to 30 mg are administered daily for a five-day course, such courses repeated as needed with rest period of one or more weeks interposed between courses of therapy. For leukemias, twice weekly intramuscular injections are administered in doses of 30 mg/m². For mycosis fungoides, weekly intramuscular injections of doses of 50 mg or, alternatively, of 25 mg are administered twice weekly. In accordance with the methods described herein, a compound and/or a prodrug compound of the invention is co-administered with methotrexate administered at such doses (or at lower doses). 5-Methyl-6-[[(3,4,5-trimethoxyphenyl)-amino]methyl]-2,4-quinazolined iamine (commonly known as trimetrexate) is another antifolate drug that can be co-administered with a compound and/or a prodrug compound of the invention.

Inorganic arsenates useful in the practice of the methods described herein include but are not limited to arsenic trioxide (Trisenox). In accordance with the methods described herein a compound and/or a prodrug compound of the invention is co-administered with an inorganic arsenate to treat cancer. In one version, the cancer is refractory acute promyelocytic leukemia (APL).

Microtubule inhibitors (as used herein, a “microtubule inhibitor” is any agent that interferes with the assembly or disassembly of microtubules) useful in the practice of the methods described herein include but are not limited to vincristine (Oncovin), vinblastine (Velban), paclitaxel (Taxol, Paxene), vinorelbine (Navelbine), docetaxel (Taxotere), epothilone B or D or a derivative of either, and discodermolide or its derivatives. In accordance with the methods described herein a compound and/or prodrug of the invention is co-administered with a microtubule inhibitor to treat cancer. In one version, the cancer is ovarian cancer, breast cancer, non-small cell lung cancer, Kaposi's sarcoma, and metastatic cancer of breast or ovary origin. As one example, the compound 22-oxo-vincaleukoblastine, also commonly known as vincristine, is an alkaloid obtained from the common periwinkle plant (Vinca rosea, Linn.) and is useful in the treatment of acute leukemia. It has also been shown to be useful in combination with other oncolytic agents in the treatment of Hodgkin's disease, lymphosarcoma, reticulum-cell sarcoma, rhabdomyosarcoma, neuroblastoma, and Wilm's tumor. Vincristine is administered in weekly intravenous doses of 2 mg/m² for children and 1.4 mg/m² for adults. In accordance with the methods described herein, a compound and/or prodrug compound of the invention is co-administered with vincristine administered at such doses. In one version, a compound and/or prodrug compound of the invention is not administered prior to treatment with a microtubule inhibitor, such as a taxane, but rather, administration of a compound and/or prodrug compound of the invention is administered simultaneously with or within a few days to a week after initiation of treatment with a microtubule inhibitor.

Modifiers useful in the practice of the methods described herein include but are not limited to Leucovorin (Wellcovorin), which is used with other drugs such as 5-fluorouracil to treat colorectal cancer. In accordance with the methods described herein a compound and/or prodrug compound of the invention is co-administered with a modifier and another anti-cancer agent to treat cancer. In one version, the cancer is colon cancer. In one version, the modifier is a compound that increases the ability of a cell to take up glucose, including but not limited to the compound N-hydroxyurea. N-hydroxyurea has been reported to enhance the ability of a cell to take up 2-deoxyglucose (see the reference Smith et al., 1999, Cancer Letters 141: 85, incorporated herein by reference), and administration of N-hydroxyurea at levels reported to increase 2-deoxyglucose uptake or to treat leukemia together with administration of 2-deoxyglucose and a compound of the invention is one version of the therapeutic methods provided herein. In another such version, a compound and/or prodrug compound of the invention is co-administered with nitric oxide or a nitric oxide precursor, such as an organic nitrite or a spermineNONOate, to treat cancer, as the lafter compounds stimulate the uptake of glucose.

Nitrosoureas useful in the practice of the methods described herein include but are not limited to procarbazine (Matulane), lomustine, CCNU (CeeBU), carmustine (BCNU, BICNU, Gliadel Wafer), and estramustine (Emcyt). In accordance with the methods described herein a compound and/or prodrug compound and/or prodrug compound of the invention is co-administered with a nitrosourea to treat cancer. In one version, the cancer is prostate cancer or glioblastoma, including recurrent glioblastoma multiforme.

Nucleoside analogs useful in the practice of the methods described herein include but are not limited to mercaptopurine, 6-MP (Purinethol), fluorouracil, 5-FU (Adrucil), thioguanine, 6-TG (Thioguanine), hydroxyurea (Hydrea), cytarabine (Cytosar-U, DepoCyt), floxuridine (FUDR), fludarabine (Fludara), azacytidine (Vidaza), pentostatin (Nipent), cladribine (Leustatin, 2-CdA), gemcitabine (Gemzar), and capecitabine (Xeloda). In accordance with the methods described herein a compound and/or prodrug compound of the invention is co-administered with a nucleoside analog to treat cancer. In one version, the cancer is B-cell lymphocytic leukemia (CLL), hairy cell leukemia, adenocarcinoma of the pancreas, metastatic breast cancer, non-small cell lung cancer, or metastatic colorectal carcinoma. As one example, the compound 5-fluoro-2,4(1H,3H)-pyrimidinedione, also commonly known as 5-fluorouracil, is an antimetabolite nucleoside analog effective in the palliative management of carcinoma of the colon, rectum, breast, stomach, and pancreas in patients who are considered incurable by surgical or other means. 5-Fluorouracil is administered in initial therapy in doses of 12 mg/m² given intravenously once daily for 4 successive days with the daily dose not exceeding 800 mg. If no toxicity is observed at any time during the course of the therapy, 6 mg/kg are given intravenously on the 6th, 8th, 10th, and 12th days. No therapy is given on the 5th, 7th, 9th, or 11th days. In poor risk patients or those who are not in an adequate nutritional state, a daily dose of 6 mg/kg is administered for three days, with the daily dose not exceeding 400 mg. If no toxicity is observed at any time during the treatment, 3 mg/kg can be given on the 5th, 7th, and 9th days. No therapy is given on the 4th, 6th, or 8th days. A sequence of injections on either schedule constitutes a course of therapy. In accordance with the methods described herein, a compound and/or prodrug compound of the invention is co-administered with 5-FU administered at such doses or with the prodrug form Xeloda with correspondingly adjusted doses. As another example, the compound 2-amino-1,7-dihydro-6H-purine-6-thione, also commonly known as 6-thioguanine, is a nucleoside analog effective in the therapy of acute non-pymphocytic leukemias. 6-Thioguanine is orally administered in doses of about 2 mg/kg of body weight per day. The total daily dose can be given at one time. If after four weeks of dosage at this level there is no improvement, the dosage can be cautiously increased to 3 mg/kg/day. In accordance with the methods described herein, a compound and/or prodrug compound of the invention is co-administered with 6-TG administered at such doses (or at lower doses).

Osteoclast inhibitors useful in the practice of the methods described herein include but are not limited to pamidronate (Aredia). In accordance with the methods described herein a compound and/or prodrug compound of the invention is co-administered with an osteoclast inhibitor to treat cancer. In one version, the cancer is osteolytic bone metastases of breast cancer, and one or more additional anti-cancer agents are also co-administered with a compound and/or prodrug compound of the invention.

Platinum compounds useful in the practice of the methods described herein include but are not limited to cisplatin (Platinol) and carboplatin (Paraplatin). In accordance with the methods described herein a compound and/or prodrug compound of the invention is co-administered with a platinum compound to treat cancer. In one version, the cancer is metastatic testicular cancer, metastatic ovarian cancer, ovarian carcinoma, and transitional cell bladder cancer. As one example, the compound cis-Diaminedichloroplatinum (II), commonly known as cisplatin, is useful in the palliative treatment of metastatic testicular and ovarian tumors, and for the treatment of transitional cell bladder cancer which is not amenable to surgery or radiotherapy. Cisplatin, when used for advanced bladder cancer, is administered in intravenous injections of doses of 50-70 mg/m² once every three to four weeks. In accordance with the methods described herein, a compound and/or prodrug compound of the invention is co-administered with cisplatin administered at these doses (or at lower doses). One or more additional anti-cancer agents can be co-administered with the platinum compound and a compound and/or prodrug compound of the invention. As one example, Platinol, Blenoxane, and Velbam can be co-administered with a compound and/or a prodrug compound of the invention. As another example, Platinol and Adriamycin can be co-administered with a compound and/or a prodrug compound of the invention.

Retinoids useful in the practice of the methods described herein include but are not limited to tretinoin, ATRA (Vesanoid), alitretinoin (Panretin), and bexarotene (Targretin). In accordance with the methods described herein a compound and/or a prodrug compound of the invention is co-administered with a retinoid to treat cancer. In one version, the cancer is a cancer selected from the group consisting of APL, Kaposi's sarcoma, and T-cell lymphoma.

Topoisomerase 1 inhibitors useful in the practice of the methods described herein include but are not limited to topotecan (Hycamtin) and irinotecan (Camptostar). In accordance with the methods described herein a compound and/or a prodrug compound of the invention is co-administered with a topoisomerase 1 inhibitor to treat cancer. Topoisomerase inhibitors and prodrugs thereof useful in the practice of the methods of the present invention are provided in the reference Matteucci et al., U.S. Patent Application No. 60/629,723. In one version, the cancer is metastatic carcinoma of the ovary, colon, or rectum, or small cell lung cancer. As noted above, however, in one version of the methods described herein, administration of a compound and/or a prodrug compound of the invention either precedes or follows, or both, administration of a topoisomerase 1 inhibitor but is not administered concurrently therewith.

Topoisomerase 2 inhibitors useful in the practice of the methods described herein include but are not limited to etoposide, VP-16 (Vepesid), teniposide, VM-26 (Vumon), and etoposide phosphate (Etopophos). In accordance with the methods described herein a compound and/or prodrug compound of the invention is co-administered with a topoisomerase 2 inhibitor to treat cancer. In one version, the cancer is a cancer selected from the group consisting of refractory testicular tumors, refractory acute lymphoblastic leukemia (ALL), and small cell lung cancer. As noted above, however, in one version of the methods described herein, administration of a compound and/or a prodrug of the invention either precedes or follows, or both, administration of a topoisomerase 2 inhibitor but is not administered concurrently therewith.

Tyrosine kinase inhibitors useful in the practice of the methods described herein include but are not limited to imatinib (Gleevec). In accordance with the methods described herein a compound and/or a prodrug compound of the invention is co-administered with a tyrosine kinase inhibitor to treat cancer. In one version, the cancer is CML or a metastatic or unresectable malignant gastrointestinal stromal tumor.

Lonidamine analogs useful in the practice of the present invention are provides in the reference PCT Pat. Appl. Nos. PCT/US2005/026929 and PCT/US2005/027092 and PCT/US2005/024434.

Thus, described herein are methods of treating cancer in which a compound and/or a prodrug compound of the invention or a pharmaceutically acceptable salt thereof and one or more additional anti-cancer agents are administered to a patient. Specific versions of such other anti-cancer agents include without limitation 5-methyl-6-[[(3,4,5-trimethoxyphenyl)amino]-methyl]-2,4-quinazolinediamine or a pharmaceutically acceptable salt thereof, (8S,10S)-10-(3-amino-2,3,6-trideoxy-alpha-L-lyxo-hexopyranosyl)oxy]-8-glycoloyl-7,8,9,10-tetrahydro-6,8,11-trihydroxy-1-methoxy-5,12-naphthacenedione or a pharmaceutically acceptable salt thereof; 5-fluoro-2,4(1H,3H)-pyrimidinedione or a pharmaceutically acceptable salt thereof; 2-amino-1,7-dihydro-6H-purine-6-thione or a pharmaceutically acceptable salt thereof; 22-oxo-vincaleukoblastine or a pharmaceutically acceptable salt thereof; 2-bis[(2-chloroethyl)amino]tetrahydro-2H-1,3,2-oxazaphosphorine, 2-oxide, or a pharmaceutically acceptable salt thereof; N-[4-[[(2,4-diamino-6-pteridinyl)methyl]-methylamino]benzoyl]-L-glutamic acid, or a pharmaceutically acceptable salt thereof; or cisdiamminedichloro-platinum (II).

Although the present invention has been described in detail with reference to specific embodiments, those of skill in the art will recognize that modifications and improvements are within the scope and spirit of the invention, as set forth in the claims which follow. All publications and patent documents (patents, published patent applications, and unpublished patent applications) cited herein are incorporated herein by reference as if each such publication or document was specifically and individually indicated to be incorporated herein by reference. Citation of publications and patent documents is not intended as an admission that any such document is pertinent prior art, nor does it constitute any admission as to the contents or date of the same. The invention having now been described by way of written description and example, those of skill in the art will recognize that the invention can be practiced in a variety of embodiments and that the foregoing description and illustrative methods are for purposes of exemplification and not limitation of the following claims.

### EXAMPLES

### Example 1

Synthesis of

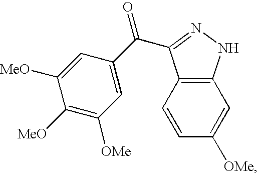

intermediates thereto, and derivatives thereof is provided below.

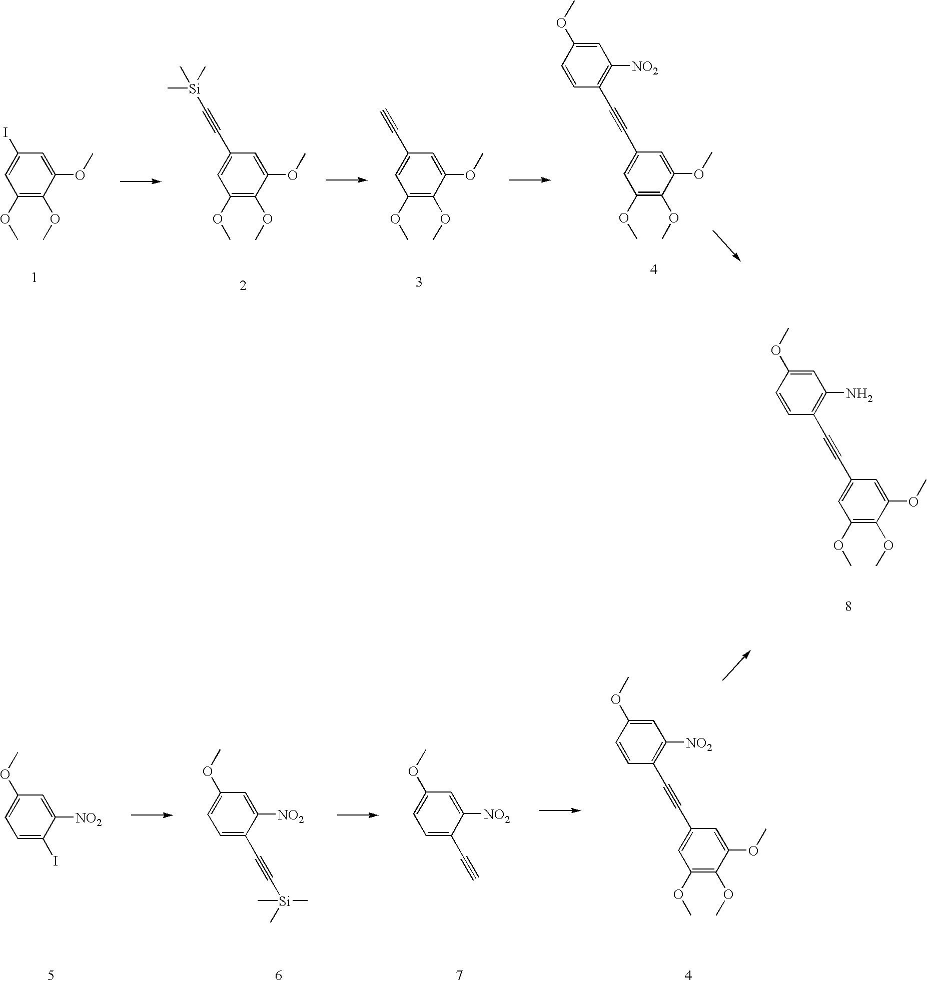

Compound 2 A 50-mL two-necked round-bottomed flask equipped with a septum, a stir-bar, and a water condenser topped with a nitrogen inlet was charged with a mixture of compound 1 (588 mg, 2.0 mmol), PdCl₂(PPh₃)₂ (70 mg, 5.0 mol %), CuI (19 mg, 5.0 mol %) of, and triethylamine (TEA, 15 mL). Trimethylsilylacetylene (0.47 mL, 3.4 mmol 1.7 eq.) was added to it at room temperature (rt). After 30 min the solution was heated to 50° C. under nitrogen. After complete consumption of starting material (monitored by thin layer chromatography (TLC)) the mixture was cooled to rt and gravity filtered, and the solid was washed with dichloromethane (DCM, 10 mL). The filtrate was concentrated under reduced pressure to give a crude product, which was separated by flash chromatography on silica gel (Hex:AcOEt=100:10 (v/v)) to give 470 mg of compound 2 (89%).

Compound 3 To a solution containing compound 2 (470 mg), water (1 mL), and THF (18 mL) was added 1 M tetrabutylammonium fluoride solution (5.3 mL) at 0° C. The mixture was stirred at rt overnight. After the solvent was removed under reduced pressure, DCM (20 mL) was added. The organic phase was washed with water and dried over Na₂SO₄ and concentrated under reduced pressure. The residue was chromatographed on silica gel (Hex:AcOEt=100:15 (v/v)) to give compound 3 (250 mg).

Compound 4 To a solution containing compound 3 (240 mg, 1.25 mmol) and 4-iodo-3-nitroanisole (345 mg, 1.24 mmol) were added PdCl₂(PPh₃)₂ (44 mg, 5.0 mol %), and CuI (12 mg, 5.0 mol %) in TEA (15 mL). The mixture was stirred at 55° C. for 3 h, cooled, and filtered. The filtrate was concentrated under reduced pressure. Chromatography of the residue on silica gel (Hex:AcOEt=100:40 (v/v)) gave 380 mg (88%) of compound 4.

### Example 2

Compound 6 Compound 6 was prepared from compound 5 (3.35 g, 12 mmol), trimethylsilylacetylene (3.3 mL, 24 mmol), PdCl₂(PPh₃)₂ (0.42 g, 5.0 mol %), and CuI (0.114 g, 5.0 mol %) in TEA (70 mL). The reaction mixture was diluted with EtOAC and filtered through a silica gel bed, the organic layer was washed with water, and dried over Na2SO4. The dried organic layer was concentrated and purfied by chromatographic separation using (Hex:AcOEt=100:10 (v/v)) gave 1.66 g (55%) of compound 6.

Compound 7 Compound 7 was prepared from 1.66 g (6.64 mmol) of compound 6 in 3 mL water, 70 mL THF, and 20.0 mL (20 mmol) of 1 M tetrabutylammonium fluoride solution The reaction mixture was diluted with EtOAC and filtered through a silica gel bed, the organic layer was washed with water, and dried over Na₂SO₄. The dried organic layer was concentrated and purified by and chromatographic separation using (Hex:AcOEt=100:15 (v/v)) gave 1.08 g (91%) of compound 7.

Compound 4 can also be prepared by a similar procedure from compound 7 (100 mg, 0.56 mmol), 157 mg 5-iodo-1,2,3-trimethoxybenzene (0.53 mmol), PdCl₂(PPh₃)₂ (19 mg, 5.0 mol %), and CuI (5.1 mg, 5.0 mol %) in TEA (8 mL). The reaction mixture was diluted with EtOAC and filtered through a silica gel bed, the organic layer was washed with water, and dried over Na₂SO₄. The dried organic layer was concentrated and purified by chromatographic separation using (Hex:AcOEt=100:40 (v/v)), 140 mg (76%) of compound 4 was obtained.

Compound 8 Compound 4 (140 mg, 0.41 mmol) was suspended in EtOH 95% (15 mL) and heated at 80° C. for 30 min. To this mixture concentrated HCl (0.017 mL) and iron powder (230 mg, 8.3 mmol) were added. The reaction mixture was refluxed for 2 h, cooled, and filtered. The filtrate was concentrated under reduced pressure. Chromatography of the residue on silica gel (Hex:AcOEt=100:40 (v/v)) gave 62 mg (49%) of compound 8.

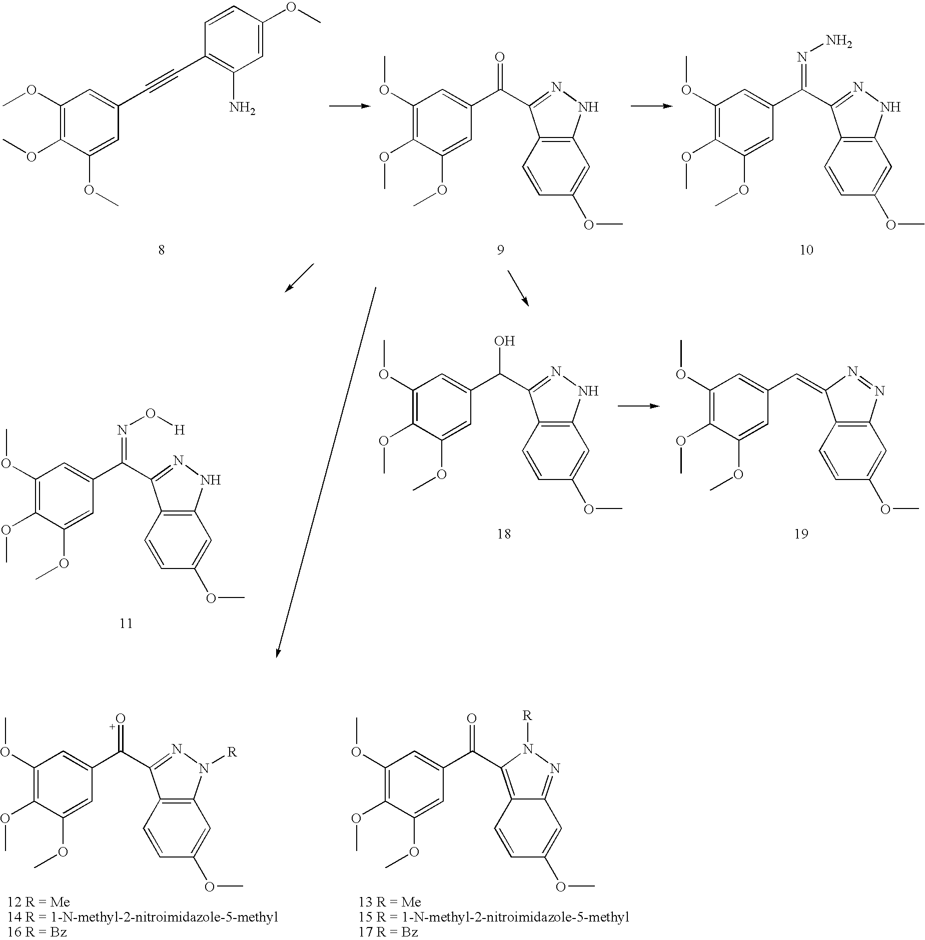

### Example 3

Compound 9 To a solution containing compound 8 (170 mg, 0.54 mmol) in 1:2 water/acetone (10 mL) of was added dropwise 10% HCl (3 mL). The resulting mixture was cooled down to −10° C. A solution of NaNO₂ (56 mg, 0.81 mmol) in water (1 mL) was added to the reaction mixture and stirred for 30 min at −10 to −5° C. Water (50 mL) was added; the reaction mixture was warmed to rt, stirred for 30 min at rt, and extracted with AcOEt (15 mL×2). The organic phase was washed with 10% NaHCO₃ and water, dried over Na₂SO₄, and concentrated under reduced pressure. Chromatography (Hex:AcOEt=100:40 (v/v)) of the residue on silica gel afforded 110 mg (60%) of compound 9.

### Example 4

Compound 10 A solution containing compound 9 (5 mg) and hydrazine (5 mg) in EtOH (1 mL) was refluxed until starting material (monitored by TLC) disappeared. The solvent was removed under reduced pressure. The residue was dissolved in DCM and purified by preparative TLC (Hex:AcOEt=1:1 (v/v)) to give compound 10.

### Example 5

Compound 11 A solution containing compound 9 (5 mg) and hydroxylamine hydrochloride (7 mg) in EtOH (3 mL) was refluxed overnight. The solvent was removed under reduced pressure. The residue was dissolved in DCM and purified by preparative TLC (Hex:AcOEt=1:2 (v/v)) to give compound 11.

### Example 6

Compounds 12 and 13 To a solution containing compound 9 (10 mg) and CH₃I (6 mg) in 3 mL dry acetone was added K₂CO₃ (30 mg). The mixture was refluxed for 2 h and filtered. The filtrate was concentrated under reduced pressure. The residue was dissolved in DCM and purified by preparative TLC (Hex:AcOEt=1:1 (v/v)) to give compounds 12 and 13.

### Example 7

Compounds 14 and 15 Novel prodrugs 14 and 15 of this invention can be synthesized following the procedure described for the synthesis of compounds 12 and 13 by reacting compound 9 with

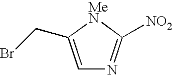

instead of CH₃I.

### Example 8

Compounds 16 and 17 To a solution containing compound 9 (10 mg) and benzyl bromide (10 mg) in 3 mL dry acetone was added K₂CO₃ (30 mg). The mixture was refluxed for 2 h and filtered. The filtrate was concentrated under reduced pressure. The residue was dissolved in DCM and purified by preparative TLC (Hex:AcOEt=1:1 (v/v)) to give compounds 16 and 17.

### Example 9

Compound 18 A solution containing compound 9 (5 mg) and NaBH₄ (1 mg) in ETOH (0.5 mL) was stirred for 3 h at rt. The solvent was removed under reduced pressure. The residue was dissolved in DCM, washing with water. The organic phase was dried over Na₂SO₄, concentrated, and purified by preparative TLC (Hex:AcOEt=1:2 (v/v)) to give compound 18.

### Example 10

Compound 19 To a solution containing compound 16 (5 mg) of in dry toluene (1 mL) p-TsOH (0.5 mg) was added. The reaction mixture was heated at 80° C. overnight. The solvent was removed under reduced pressure. The residue was dissolved in DCM (3 mL) and TEA (0.1 mL), washing with water. The organic phase was dried over Na₂SO₄, concentrated, and purified by preparative TLC (Hex:AcOEt=1:2 (v/v)) to give compound 19.

### Example 11

Example 11 provides methods for synthesizing Compound 22 starting from compound 9.

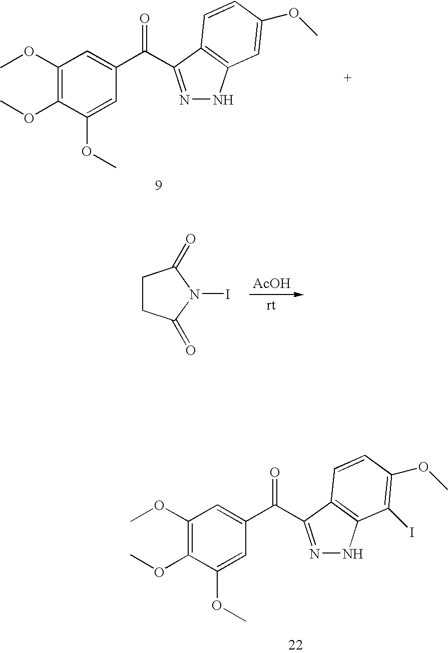

To a solution of N-iodosuccinimide (45 mg, 0.2 mmol, 1.0 eq.) in AcOH (1 mL) was added to a solution of 9 (68 mg, 0.2 mmol, 1.0 eq.) in AcOH (2 mL) at rt. The mixture was stirred at rt for 2 hr, diluted with 8 mL water and extracted with EtOAc (10 mL×3). Combined organic layers were washed until their pH was 7 using 10% NaHCO₃, and dried over Na₂SO₄ and concentrated under reduced pressure. The residue was crystallized from MeOH to yield 75 mg (80%) of compound 22.

### Example 12

Example 12 provides methods for synthesizing Compound 30 starting from compound 22.

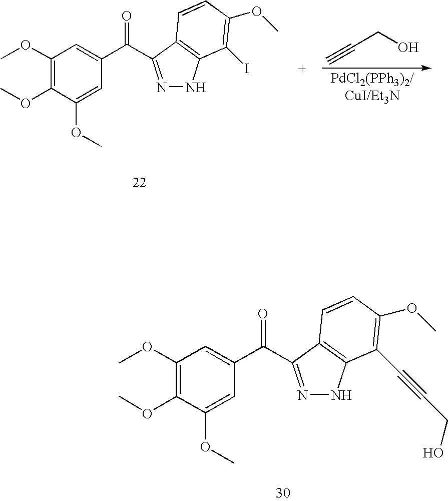

A mixture of compound 22 (24 mg), PdCl₂(PPh₃)₂ (3.5 mg) and CuI (1 mg) in Et₃N (2 mL) was thrice degassed and exchanged with Ar followed by addition of propargylalcohol (5.6 mg) at room temperature (rt) and stirred at 55° C. for 4 h and filtered. The filtrate was concentrated under reduced pressure and the residue was separated employing flash chromatography on silica gel using as eluent 2:1 (v/v) Hexanes/EtOAc to yield 8 mg (39%) of compound 30.

### Example 13

Example 13 provides a method of synthesizing compounds 54 and 55 starting from compound 30.

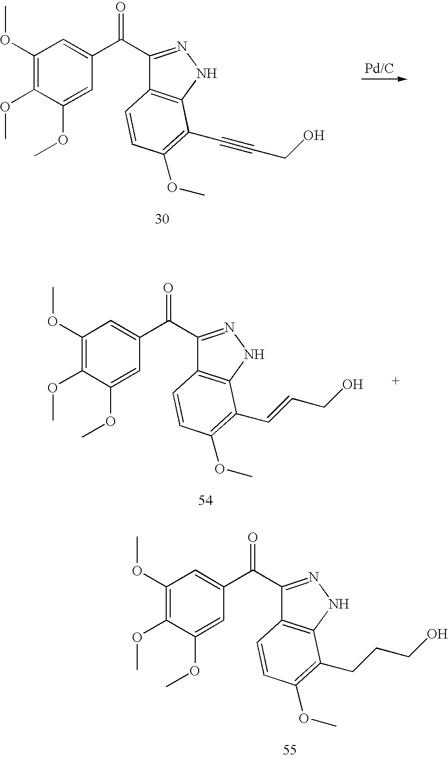

2 mg of 10% Pd/C was added to a solution of 18 mg of 30 in MeOH (20 mL) in an autoclave, purged with hydrogen thrice, stirred under 50 psi hydrogen at rt overnight and filtered. The filtrate was concentrated reduced pressure, the residue dissolved in DCM and purified by preparative TLC to yield compounds 54 and 55 (Hex:AcOEt=1:1 (v/v)).

### Example 14

Example 14 provides a method of synthesizing compound 53.

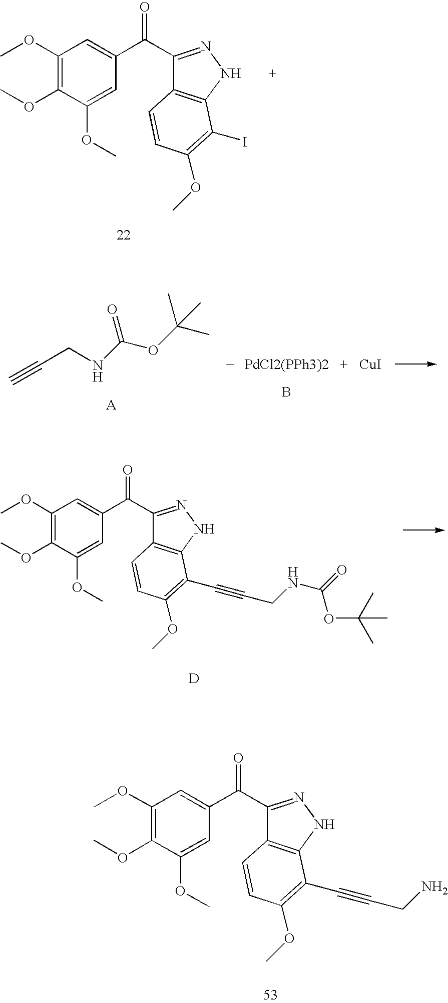

A mixture of compound 22 (93 mg), PdCl₂(PPh₃)₂ (14 mg) and CuI (3.8 mg) in Et₃N (2 mL) was thrice degassed and exchanged with Ar followed by addition of A (62 mg) at room temperature (rt) and stirred at 55° C. for 3 h and filtered. The filtrate was concentrated under reduced pressure and the residue was separated employing flash chromatography on silica gel using as eluent 1:1 (v/v) Hexanes/EtOAc to yield 30 mg of compound D.

A mixture of compound D (25 mg) in HCl (4M, 5 mL) in dioxane was stirred for 20 min (monitored by thin layer chromatography) at rt. After the solvent was removed under reduced pressure, AcOEt (10 mL) was added. The organic phase was washed with 10% NaHCO₃ and water, and dried over Na₂SO₄ and concentrated under reduced pressure. The residue was chromatographed on silica gel (Hex:AcOEt=100:50 (V/V) to give compound 53.

### Example 15

Example 15 provides a method of synthesizing compound 56.

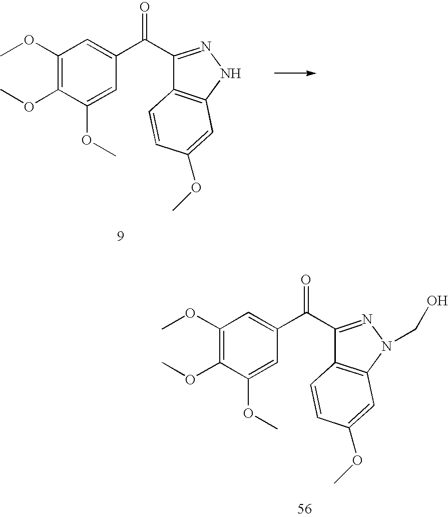

A solution of K₂CO₃ (20 mg) in 1 mL water was added to a solution of compound 9 (171 mg) in 10:1 EtOH/THF (10 mL), formaldehyde (0.1 mL, 37%). After the mixture was stirred at rt for 12 h, DCM (20 mL) was added. The organic phase was washed with water and dried over Na₂SO₄ and concentrated under reduced pressure. The residue was separated by column chromatography on silica gel using as eluent 100:50 (v/v) Hex:AcOEt to give compound 53 (55 mg).

### Example 16

Example 16 provides a method of synthesizing compound 57.

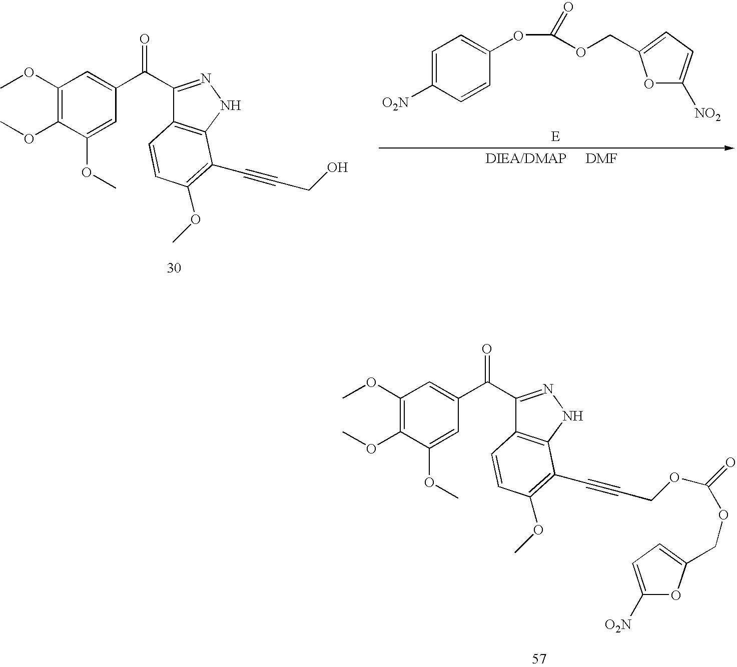

DMAP (1 mg) and DIEA (10 μL) were added to a solution of compound 30 (12 mg) and compound E (18 mg) in 3 mL dry DMF at rt. After the mixture was stirred overnight, water (10 mL) was added and extracted with AcOEt (10 mL×2). The organic phase was washed with 10% NaHCO₃ and water, dried over Na₂SO₄, and concentrated under reduced pressure. Chromatography (Hex:AcOEt=100:70 (V/V)) of the residue on silica gel afforded compound 57.

### Example 17

Example 17 provides a method of synthesizing compounds 58-60.

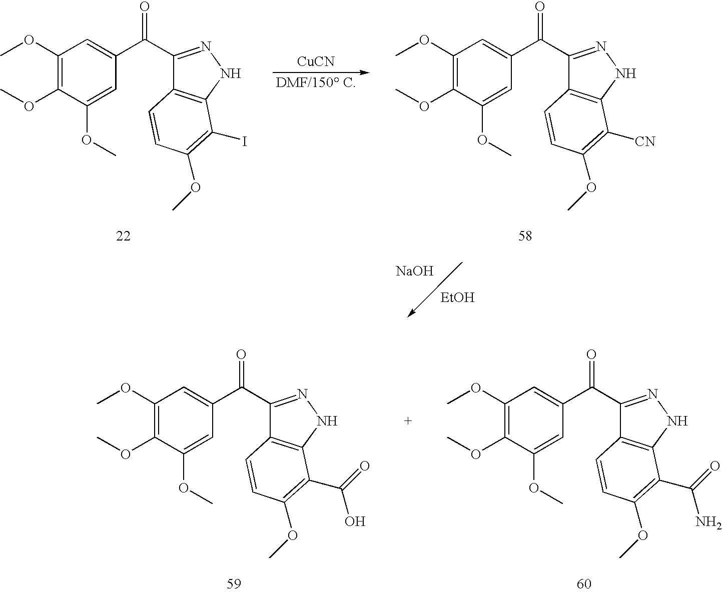

A mixture of compound 22 (47 mg), and CuCN (22.5 mg) in dry DMF (4 mL) was thrice degassed and exchanged with Ar. After the mixture was heated at 150° C. for 8 hrs and cooled to rt, water (10 mL) and DCM (20 mL) were added. The mixture was filtered, and the solid was washed with DCM (10 mL). The filtrate was concentrated under reduced pressure to give a crude product, which was separated by flash chromatography on silica gel (Hex:AcOEt=100:50 (V/V)) to give compound 58.

NaOH (3 mL, 1 M) was added to a solution of compound 58 (10 mg) in EtOH (10 mL). Then the mixture was refluxed for overnight. After the solvent was removed under reduced pressure, 1% HCl (2 mL) and AcOEt (10 mL) were added. The organic phase was washed with water, and dried over Na₂SO₄ and concentrated under reduced pressure. The residue was dissolved in DCM and purified by preparative TLC using AcOEt as eluent to give compound 59 and 60.

### Example 18

Example 18 provides a method of synthesizing compound 61.

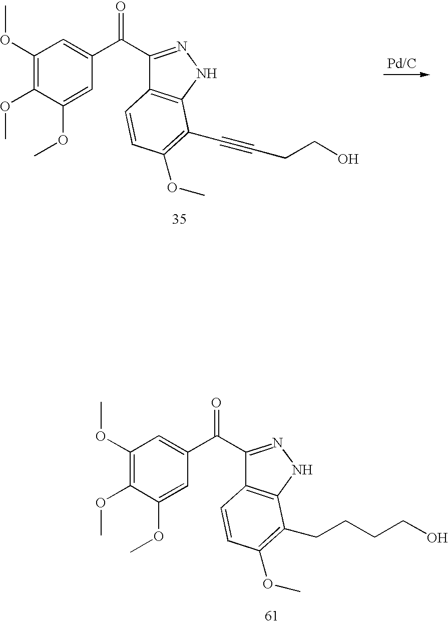

To a solution of 35 (10 mg) in MeOH (8 mL) was added 10% Pd/C (1 mg), the air purged with hydrogen thrice, and stirred under hydrogen at rt overnight and filtered. The filtrate was concentrated under reduced pressure, the residue dissolved in DCM and purified by preparative TLC employing 1:1 Hex:AcOEt to yield compounds 61.

### Example 19

Example 19 provides a method of synthesizing compound 61.

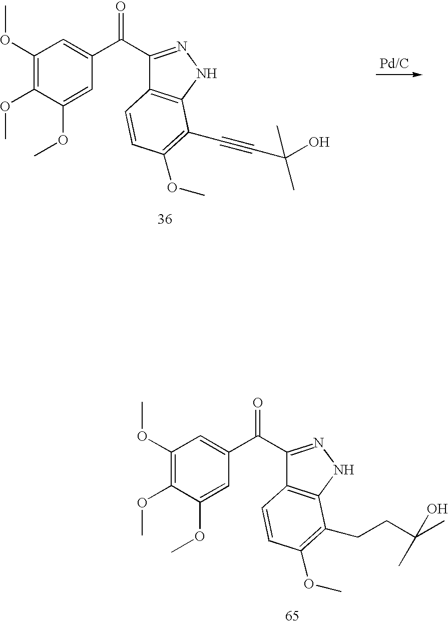

To a solution of 36 (15 mg) in MeOH (8 mL) was added 10% Pd/C (2 mg), the air purged with hydrogen thrice, and stirred under hydrogen at rt overnight and filtered. The filtrate was concentrated under reduced pressure, the residue dissolved in DCM and purified by preparative TLC employing 1:1 (v/v) Hex:AcOEt to yield compounds 61.

### Example 20

Example 20 provides a method of synthesizing compound 62-64.

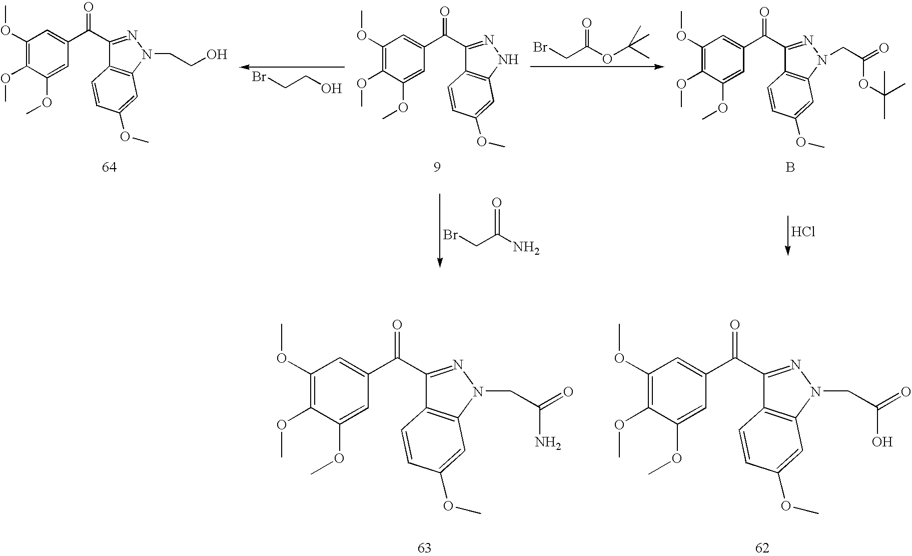

To a solution of compound 9 (68 mg) and tert-butyl bromoacetate (30 μL) in dry acetone (10 mL) was added K₂CO₃ (30 mg). The mixture was refluxed for 4 h and filtered. The filtrate was concentrated under reduced pressure and the residue separated employing flash chromatography on silica gel using as eluent 1:1 Hexanes/EtOAc to yield 44 mg of compound B.

To a solution of compound B (10 mg) in HCl in dioxane (2 mL, 4M) was stirred for 1 h (monitored by thin layer chromatography) at rt. After the solvent was removed under reduced pressure, the residue was chromatographed on silica gel (AcOEt:MeOH=100:10 (V/V) to yield compound 62 (6 mg).

To a solution of compound 9 (34 mg) and 2-bromoacetamide (14 mg) in 8 mL dry acetone was added K₂CO₃ (20 mg). The mixture was refluxed for 8 h and filtered. The filtrate was concentrated under reduced pressure and the residue washed with ether to yield compound 63 as a white solid.

To a solution containing compound 9 (51 mg) and 2-bromoethanol (12 μL) in 10 mL dry acetone was added K₂CO₃ (30 mg). The mixture was refluxed for 8 h and filtered. The filtrate was concentrated under reduced pressure and the residue separated employing flash chromatography on silica gel using as eluent 1:1 Hexanes/EtOAc to yield 21 mg of compound 64.

### Example 21

Example 21 provides a method of synthesizing compounds 66-71.

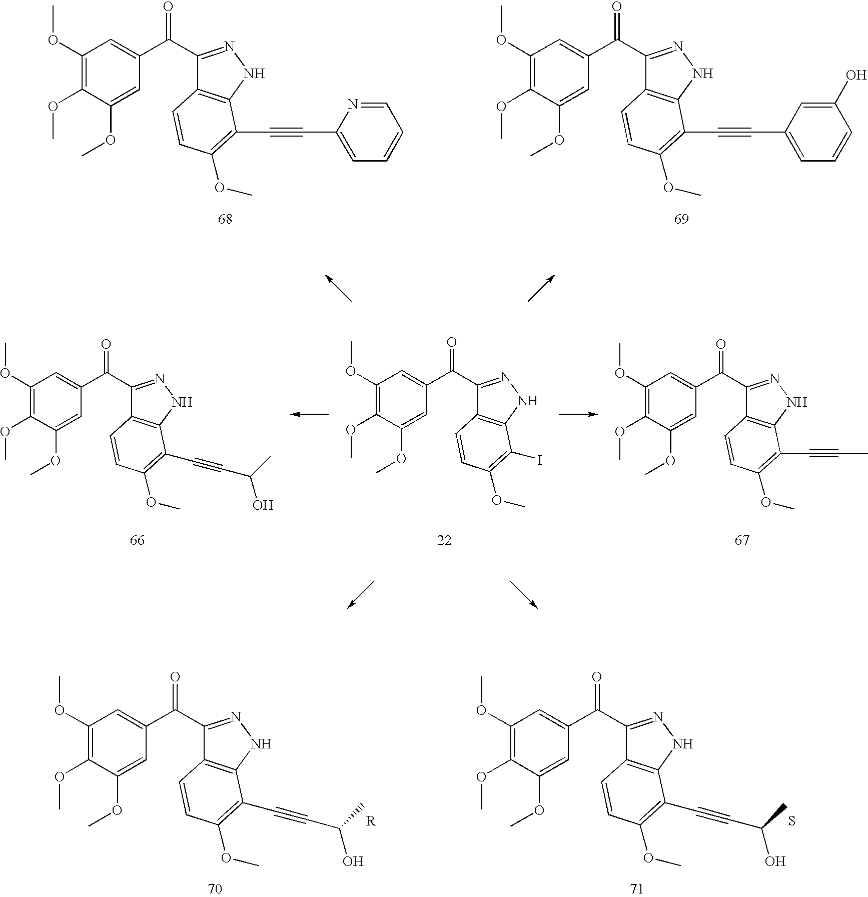

A mixture of compound 22 (46 mg), PdCl₂(PPh₃)₂ (7 mg), and CuI (2 mg) in Et₃N (6 mL) was thrice degassed and exchanged with Ar followed by addition of 3-butyn-2-ol (16 μL) at room temperature (rt) and stirred at 54° C. for 5 h and filtered. The filtrate was concentrated under reduced pressure and the residue separated employing flash chromatography on silica gel using as eluent 100:80 Hexanes/EtOAc to yield 12 mg of compound 66.

A mixture of compound 22 (46 mg), PdCl₂(PPh₃)₂ (7 mg), and CuI (2 mg) in Et₃N (6 mL) was thrice degassed and exchanged with propyne. Propyne contained in a balloon was kept in contact with the reaction mixture by using a long syringe, the system was stirred at 45° C. overnight and filtered. The filtrate was concentrated under reduced pressure and the residue was separated employing flash chromatography on silica gel using as eluent 100:40 Hexanes/EtOAc to yield 22 mg of compound 67.

A mixture of compound 22 (23 mg), PdCl₂(PPh₃)₂ (3.5 mg), and CuI (1 mg) in Et₃N (2 mL) was thrice degassed and exchanged with Ar followed by addition of 2-ethynyl pyridine (11 μL) at rt, stirred at 55° C. for 3 hr and filtered. The filtrate was concentrated under reduced pressure and the residue was separated employing flash chromatography on silica gel using as eluent 100:70 (v/v) Hexanes/EtOAc to yield 7 mg of compound 68.

A mixture of compound 22 (34.5 mg), PdCl₂(PPh₃)₂ (5.2 mg), and CuI (1.5 mg) in Et₃N (5 mL) was thrice degassed and exchanged with Ar followed by addition of 3-ethynyl phenol (16 mg) at rt and stirred at 55° C. for 4 hr and filtered. The filtrate was concentrated under reduced pressure and the residue was separated employing flash chromatography on silica gel using as eluent 100:60 (v/v) Hexanes/EtOAc to yield 20 mg of compound 69.

A mixture of compound 22 (46 mg), PdCl₂(PPh₃)₂ (7 mg), and CuI (2 mg) in Et₃N (3 mL) was thrice degassed and exchanged with Ar followed by addition of R(+)-3-butyn-2-ol (16 μL) at rt and stirred at 55° C. for 4 hr and filtered. The filtrate was concentrated under reduced pressure and the residue was separated employing flash chromatography on silica gel using as eluent 100:65 Hexanes/EtOAc to yield compound 70. Compound 71 was synthesized similarly as compound 70 after substituting R(+)-3-butyn-2-ol with S(−)-3-butyn-2-ol.

### Example 22

Example 22 provides a method of synthesizing compounds 73 and 74.

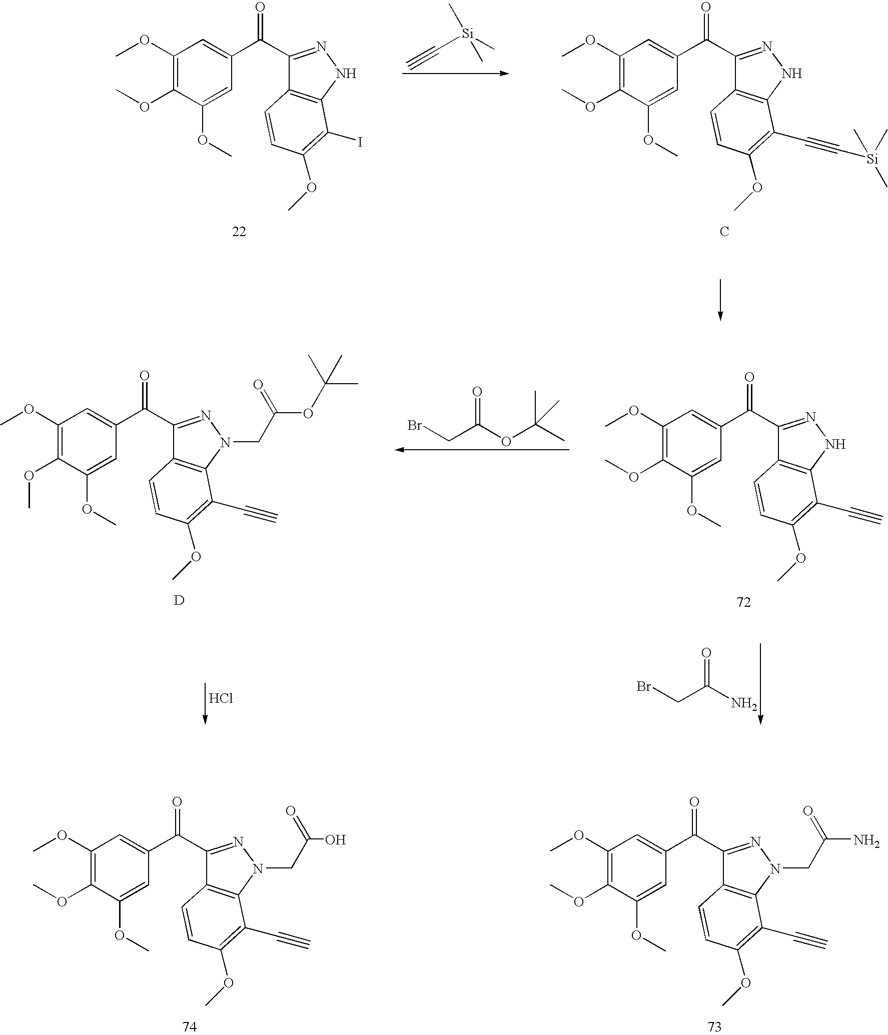

A mixture of compound 22 (468 mg), PdCl₂(PPh₃)₂ (35 mg) and CuI (10 mg) in Et₃N (70 mL) was thrice degassed and exchanged with Ar followed by addition of trimethylsilylacetylene (0.55 mL) at rt and stirred at 54° C. for 1.5 h and filtered. The filtrate was concentrated under reduced pressure to yield without further purification “crude” A (0.43 g). To a solution of compound A (430 mg), water (0.5 mL), and THF (9.5 mL) was added a solution of tetrabutylammonium fluoride in THF (1 M, 3.0 mL) at 0° C. The mixture was stirred and the temperature was allowed to rise from 0° C. to rt in 4 h. Volatiles were removed under reduced pressure followed by addition of DCM (10 mL), the organic phase washed with water, separated, dried over Na₂SO₄ and concentrated under reduced pressure. The residue was chromatographed on silica gel using as eluent 100:60 v/v Hex:AcOEt to yield compound 72 (200 mg).

To a solution of compound 72 (36 mg) and 2-bromoacetamide (14 mg) in 8 mL dry acetone was added K₂CO₃ (20 mg). The mixture was refluxed overnight and filtered. The filtrate was concentrated under reduced pressure and the residue was separated employing flash chromatography on silica gel using as eluent 100:90 Hex:AcOEt to give compound 73 (18 mg).

To a solution of compound 72 (37 mg) and tert-butyl bromoacetate (20 μL) in 8 mL dry acetone was added K₂CO₃ (20 mg). The mixture was refluxed for 2 hrs and filtered. The filtrate was concentrated under reduced pressure. The residue was separated employing flash chromatography on silica gel using as eluent 100:30 Hexanes/EtOAc to yield 32 mg white solid of compound D. A solution of compound D (15 mg) in HCl (4M, 3 mL) in dioxane was stirred at rt while the progress of the reaction was monitored by thin layer chromatography to check for the progress. After 2 h, the volatiles were removed under reduced pressure, and the residue was separated employing chromatography on silica gel using as eluent 100:10 (v/v) AcOEt:MeOH to yield compound 74.

### Example 23

Example 23 provides a method of synthesizing compound 75.

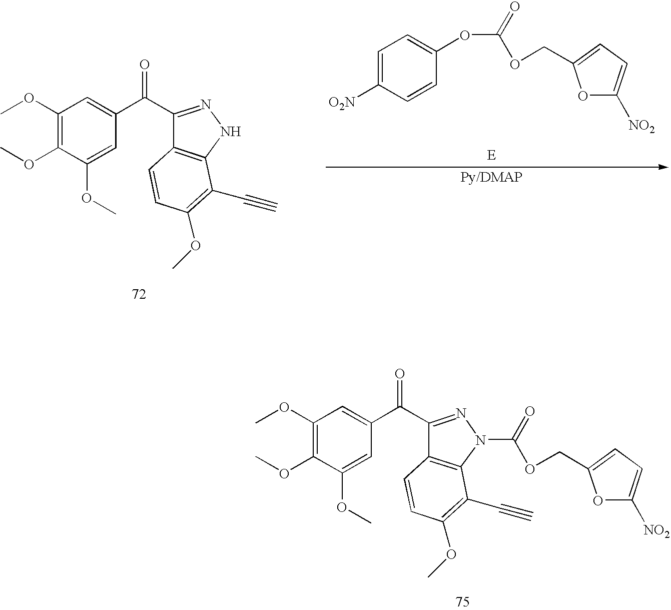

DMAP (1 mg) was added to a solution of compound 72 (18 mg) and compound E (29 mg) in 3 mL dry pyridine at rt. After the mixture was stirred overnight, water (10 mL) was added and the mixture extracted with DCM (10 mL×2). The organic phase was washed with 1% HCl, 10% NaHCO₃, water, dried over Na₂SO₄, and concentrated under reduced pressure. Chromatography (Hex:AcOEt=100:35 (v/v)) of the residue on silica gel afforded compound 75.

### Example 24

Example 24 provides a method of synthesizing compound 76.

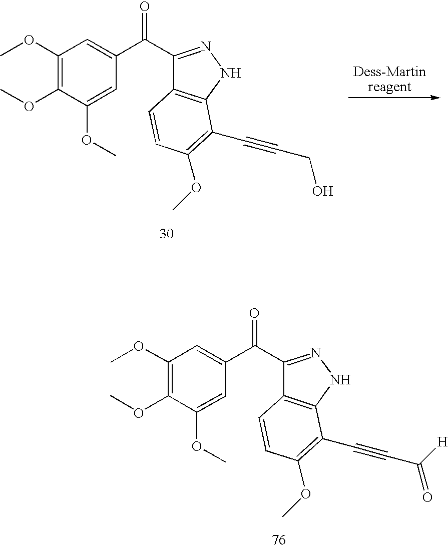

Dess-Martin reagent (0.3 M, 0.5 mL) was added to a solution of compound 30 (40 mg) in 4:1 DCM/THF (10 mL) at rt. After the solution was stirred for 1 hr, DCM (10 mL) and NaOH solution (1 M) was added until pH was equal to 7 and stirred for 5 min. The organic phase was washed with water, dried over Na₂SO₄, and concentrated under reduced pressure. Chromatography (Hex:AcOEt=100:25 (v/v)) of the residue on silica gel afforded compound 76.

### Example 25

Example 25 provides a method of synthesizing Compound 77.

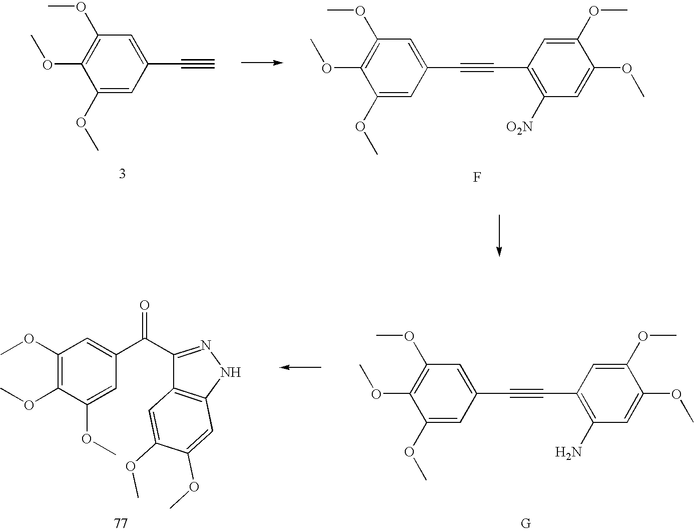

To a solution containing compound 3 (390 mg) and 4-bromo-5-nitroveratrole (500 mg) were added PdCl₂(PPh₃)₂ (77 mg, 5.0 mol %), and CuI (19 mg, 5.0 mol %) in TEA (80 mL). The mixture was stirred at 55° C. for 5 hrs, cooled, and filtered. The filtrate was concentrated under reduced pressure. Chromatography of the residue on silica gel (Hex:AcOEt=100:50 (v/v)) gave 530 mg of compound F. Compound F (530 mg) was suspended in EtOH 95% (40 mL) and heated at 88° C. for 30 min. To this mixture were added concentrated HCl (0.13 mL) and iron powder (810 mg). The reaction mixture was refluxed for 1 h, cooled, and filtered. The filtrate was concentrated under reduced pressure. Chromatography of the residue on silica gel (Hex:AcOEt=100:35 (v/v)) gave 36 mg of compound G. To a solution containing compound G (36 mg) in 1:2 water/acetone (25 mL) was added dropwise 10% HCl (0.5 mL). The resulting mixture was cooled down to −10° C. A solution of NaNO₂ (11 mg) in water (0.5 mL) was added to the reaction mixture and stirred for 30 min at −10 to −5° C. Water (50 mL) was added; the reaction mixture was warmed to rt, stirred for 30 min at rt, and extracted with AcOEt (15 mL×2). The organic phase was washed with 10% NaHCO₃ and water, dried over Na₂SO₄, and concentrated under reduced pressure. Chromatography (Hex:AcOEt=100:35 (v/v)) of the residue on silica gel afforded compound 77.

### Example 26

Example 26 provides a method of synthesizing Compound 78.

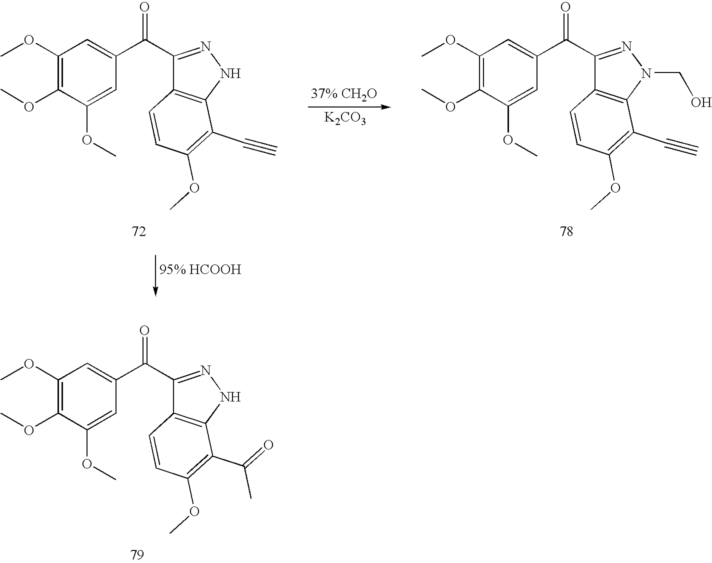

A solution of K₂CO₃ (20 mg) in 1 mL water was added to a solution of compound 72 (37 mg) in 10/1 v/v EtOH/THF (5 mL) and formaldehyde (0.05 mL 37%). After the mixture was stirred overnight at 40° C., DCM (10 mL) was added. The organic phase was washed with water and dried over Na₂SO₄ and concentrated under reduced pressure. The residue was chromatographed on silica gel (Hex:AcOEt=100:35 (V/V)) to give compound 78.

### Example 27

Example 27 provides a method of synthesizing Compound 79.

A solution of compound 72 (10 mg) in 95% formic acid was heated at 100° C. overnight. The solvent was moved under reduced pressure. The residue was chromatographed on silica gel (Hex:AcOEt=100:35 (V/V)) to give compound 79.

### Example 28

Example 28 provides a method of synthesizing Compound 80.

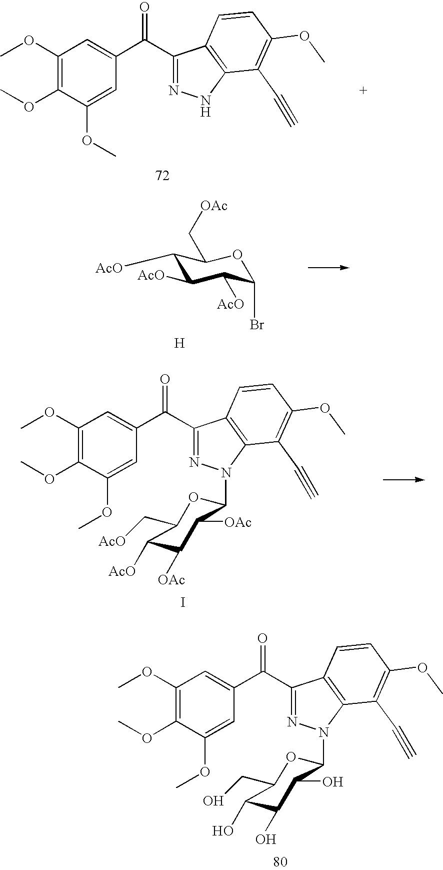

To a solution of 72 (10 mg, 0.027 mmol) in DMF (2 ml) was added NaH (1.6 mg, 0.041 mmol, 60% in oil) at room temperature. After stirring ten minutes, compound H (17 mg, 0.041 mmol) was added and the reaction was kept stirring for one hour. DMF was removed under vacuum and the residue was purified with flash silica gel chromatography column (ethyl acetate in hexane from 0 to 100%) to yield compound I (11 mg) which was characterized by MS and ¹HNMR. Compound I was dissolved in anhydrous MeOH (2 ml) and NaOMe (0.03 ml, 0.5 M in MeOH) was added at room temperature. After 0.5 hour, the reaction was passed through Amberite IR-120 (plus) resin and washed the resin with MeOH. Removal of MeOH solvent produced final compound 80 in quantitative yield.

### Example 29

Example 18 provides method for synthesis of novel a prodrug compound of the invention derived from a novel compound of the invention.

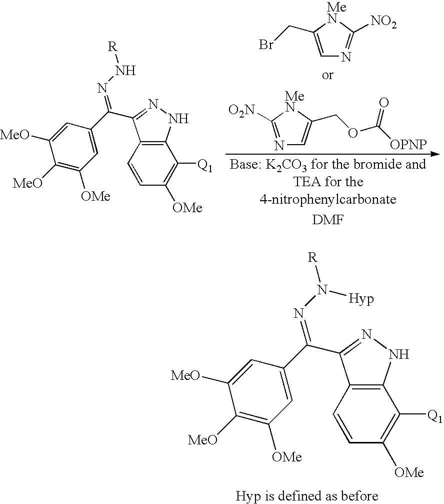

### Example 30

Example 30 provides method for synthesis of prodrug compounds of the invention employing as starting material a known tubulin binding compounds.

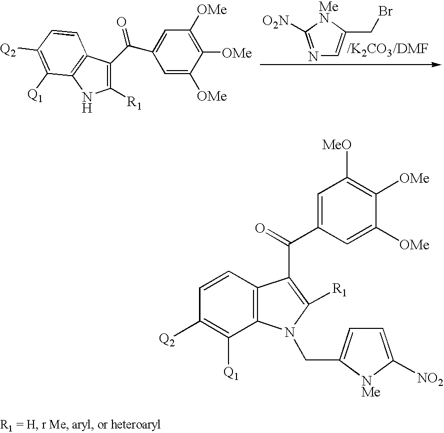

One of skill in the art can use this method can be used for the synthesis of the following prodrug compounds of the invention:

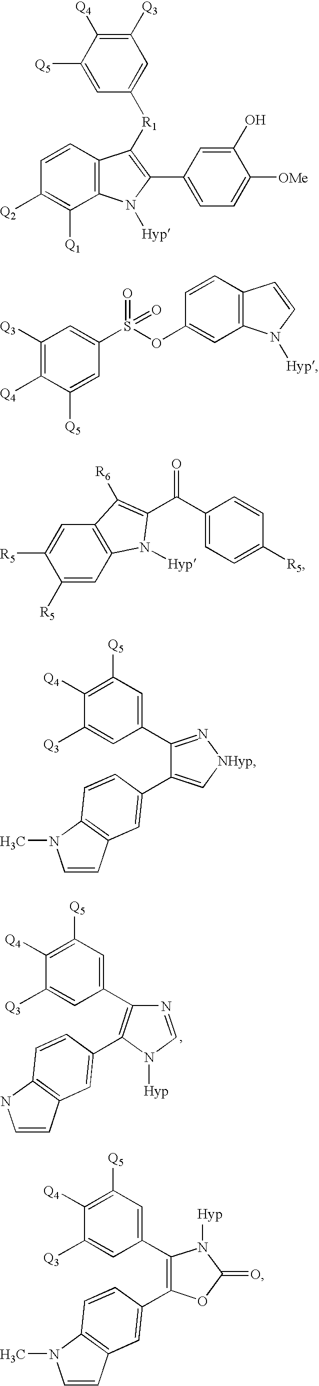

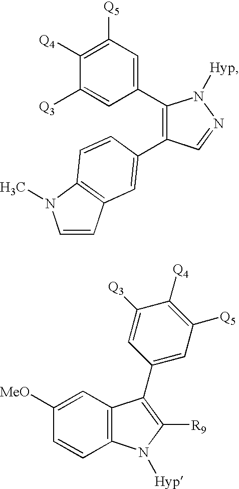

wherein Hyp′ is

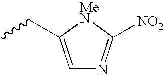

by alkylating with

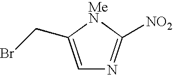

a starting material where the N-Hyp in the above formulas is replaced with —NH—.

### Example 31

Example 31 provides method for synthesis of prodrug compounds of the invention employing as starting material a known tubulin binding compounds.

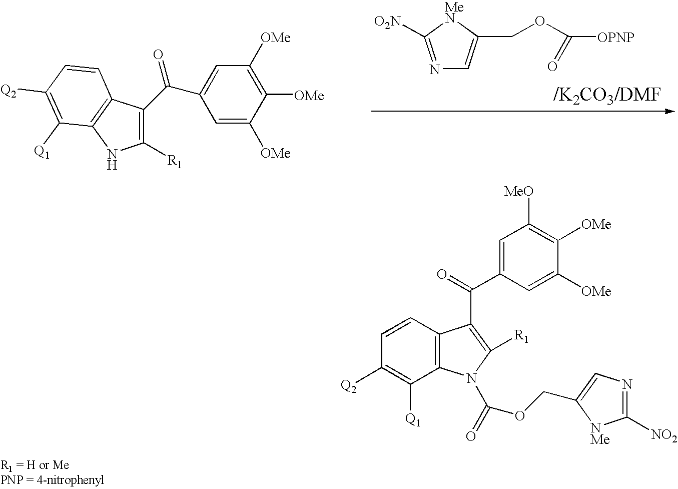

One of skill in the art can use this method can be used for the synthesis of the following prodrug compounds of the invention:

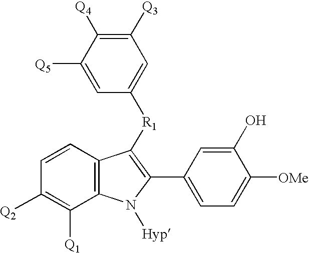

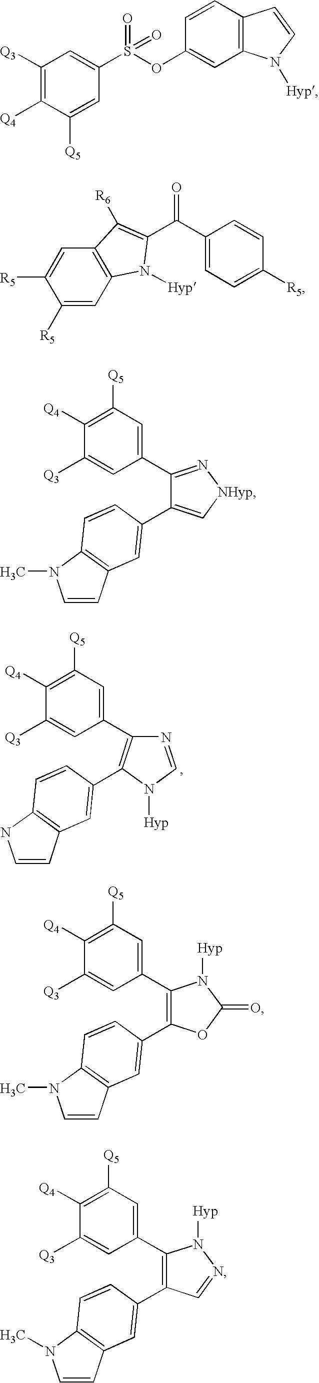

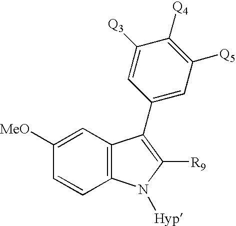

wherein Hyp″ is

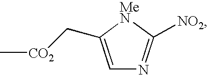

by acylating with

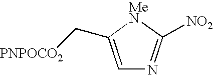

a starting material where the N-Hyp in the above formulas is replaced with —NH—. In these examples

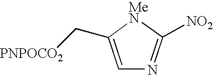

can be replaced with

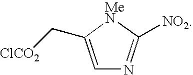

Compounds of the present invention are assayed as exemplified below:

### Example 32

To determine the effect of the compounds of the present invention on cell proliferation, the antiproliferative activity of these compounds was tested in a multi-well Alamar Blue based assay (at 2 h and 3 days). Cell growth in the presence and absence of the test compound as tabulated in Table 1 was compared, as measured by a fluorescence plate reader at excitation 550 nm and emission 590 nm (see Biosource International Inc., Tech Application Notes, Use of Alamar Blue in the measurement of Cell Viability and Toxicity, Determining IC₅₀). H460 cells (ATCC HTB-177 (NCI-H40), 4,000 cells/well/200 μl) and LNCap cells (ATCC CRL-1740, 6,000 cells/well/200 μl) were seeded in a 96 well plate in RPMI medium (Invitrogen Corporation, Carlsbad, Calif.). After 24 hours, these plates were divided into 3 groups—Control group, 2 h treatment group and 3 day treatment group.

A test compound was added to each plate in the treatment groups (2 h and 3 day) at a concentration as tabulated in Table 1 (in 50 μl of medium). In the 2 h treatment group, after 2 h the cells were rinsed to remove the test compound and incubated for 3 days, followed by staining with AlamarBlue. The cells in the 3-day treatment group were incubated for 3 days, followed by staining with AlamarBlue. In the Control group, AlamarBlue was added to the plate at (i) day 0 and (ii) day 3 and measured to establish the control reading. In all the groups, the capacity of the cells to proliferate was measured 6 hours after addition of AlamarBlue by a fluorescence plate reader at excitation 550 nm and emission 590 nm. The results of the assay are tabulated in Tables 1A and 1B.

TABLE 1A
(H460 cell line)

| | |
|---|---|
| GI₅₀ (nM) | GI₉₀ (nM) |

| Compound No. | 3 Days | 2 Hour | 3 Day | 2 Hour |
|---|---|---|---|---|
| 9 | 100 | >1000 | 316 | >1000 |
| 10 | 398 | >1000 | 1000 | >1000 |
| 11 | 630.9 | >1000 | >1000 | >1000 |
| 12 | >1000 | >1000 | >1000 | >1000 |
| 13 | >1000 | >1000 | >1000 | >1000 |
| 14 | >1000 | >1000 | >1000 | >1000 |
| 16 | >1000 | >1000 | >1000 | >1000 |
| 17 | >1000 | >1000 | >1000 | >1000 |
| 19 | >1000 | >10,000 | >1000 | >1000 |
| 20 | 100 | 630 | | |
| 21 | 40 | >10,000 | | |
| 22 | 40 | >10,000 | | |
| 23 | 100 | 630 | | |
| 24 | 501 | 10,000 | | |
| 25 | 630 | >10,000 | | |
| 26 | 630 | >10,000 | | |
| 27 | 251 | >10,000 | | |
| 28 (a and b) | >10,000 | >10,000 | | |
| 29 | 630 | >10,000 | | |
| 30 | 10 | 630 | | |
| 31 | >10,000 | >10000 | | |
| 32 | >10,000 | >10,000 | | |
| 33 | 1000 | >10,000 | | |
| 34 | 125.9 | >10,000 | | |
| 35 | 15.8 | >10,000 | | |
| 36 | 63 | >10,000 | | |
| 37 | 3.2 | 501 | | |
| 38 | 631 | >10,000 | | |
| 39 | 15.8 | | | |
| 42 | >1000 | >1000 | >1000 | >1000 |
| 43 | >10,000 | >10,000 | >1000 | >1000 |
| 44 | 1584 | >5000 | >5000 | >5000 |
| 45 | 158 | | | |
| 47 | 25 | | | |
| 48 | 630 | | | |
| 49 | 15.8 | | | |
| 50 | 20 | >10,000 | | |
| 51 (a and b) | 10 | 630 | | |
| 52 | 100 | >1000 | | |
| 53 | 158 | 630 | | |
| 54 | 2 | 501 | | |
| 55 | 7.9 | >10,000 | | |
| 56 | 50.1 | >10,000 | | |
| 57 | 10 | 100 | | |
| 58 | 15.8 | >10,000 | | |
| 59 | (794) | >1000 | | |
| 60 | (25.1) | >1000 | | |
| 61 | >1000 | >1000 | | |
| 62 | 630 | >1000 | | |
| 63 | 630 | >1000 | | |
| 64 | 630 | >1000 | | |
| 65 | >1000 | >1000 | | |
| 66 | 10 | | | |
| 67 | 639 | | | |
| 68 | 1.4 | | | |
| 69 | 1000 nM | | | |
| 70 | 10 | | | |
| 71 | 12.5 | | | |
| 72 | 1.3 | | | |
| 73 | 630 | | | |
| 74 | >1000 | | | |
| 75 | >1000 | | | |
| 77 | >1000 | | | |
| 76 | 79.4 | | | |
| 80 | >1000 | | | |
| 81 | >1000 | | | |

TABLE 1B

| Compound No. | Cell line employed | GI₅₀ (nM, 3 Day) |
|---|---|---|
| 30 | MES-SA | 0.3 |
| 30 | MES-SA/DX5 | 1.6 |
| 30 | HT29 | 1.9 |
| 30 | T47D | 2.5 |
| 35 | MES-SA | 3.2 |
| 35 | MES-SA/DX5 | 6.3 |
| 35 | HT29 | 5 |
| 35 | T47D | 10 |
| 37 | T47D | 8.9 |
| 37 | MES-SA | 3.2 |
| 37 | MES-SA/DX6 | 3.5 |
| 37 | HT29 | 5 |
| 37 | T47D | 25 |
| 39 | MES-SA | 2.5 |
| 39 | MES-SA/DX5 | 10 |
| 39 | HT29 | 12.6 |
| 39 | T47D | 200 |
| 54 | MES-SA | 3.2 |
| 54 | MES-SA/DX8 | 6.3 |
| 54 | HT29 | 5 |
| 54 | T47D | 20 |
| 55 | MES-SA | 12 |
| 55 | MES-SA/DX57 | 15.8 |
| 55 | HT29 | 10 |
| 55 | T47D | 25 |
| 66 | MESSA | 15.8 |
| 66 | MESSA/DX5 | 10 |
| 66 | HT29 | 12.6 |
| 66 | T47D | 6.3 |
| 68 | MESSA | 1.6 |
| 68 | MESSA/DX5 | 1.9 |
| 68 | HT29 | 1.6 |
| 68 | T47D | 1.6 |
| 70 | MESSA | 11.2 |
| 70 | MESSA/DX5 | 10 |
| 70 | HT29 | 12.6 |
| 70 | T47D | 6.3 |
| 71 | MESSA | 11.2 |
| 71 | MESSA/DX5 | 10 |
| 71 | HT29 | 12.6 |
| 71 | T47D | 15.8 |
| 72 | MESSA | 1.9 |
| 72 | MESSA/DX5 | 3.9 |
| 72 | HT29 | 1.6 |
| 72 | T47D | 6.3 |

### Example 33

### Cell Cycle Analysis

The effect of compounds 39 and 20 on the cell cycle was determined as follows. H460 cells (2×10⁵ cells/ml/well) were seeded in a 24 well plate. After 24 h, compound was added at various concentrations as tabulated in Table 3. The culture media were removed after 24 h, the cells were trypsinized and centrifuged. The cell pellets were resuspended in 100μl PBS buffer, after which 300 μl of ice-cold ethanol (96%) added dropwise, and the cells were incubated at 4° C. for at least 24 hr. The cells were centrifuged and the supernatant was discarded. The cell cycle staining reagent (Guava Technologies, Hayward, Calif., USA, 200 μl) was added to each well. The cells were shielded from light and incubated at room temperature for 30 min. The samples were analyzed (Guava PCA-96 instrument, Cytosoft software, Guava Technologies, 25801 Industrial Boulevard, Hayward Calif. 94545-2991, USA) to show M phase cell cycle arrest as tabulated below in Table 2.

TABLE 2
Compounds

| | 39 | | | 30 | | |
|---|---|---|---|---|---|---|
| Conc (nM) | % Gₒ/G₁ | % S | % G₂/M | % Gₒ/G₁ | % S | % G₂/M |
| 0 | 48 | 14 | 29 | 48 | 14 | 29 |
| 0.4 | 50 | 15 | 28 | 46 | 14 | 31 |
| 1.2 | 47 | 14 | 31 | 48 | 15 | 29 |
| 3.7 | 45 | 12 | 32 | 46 | 14 | 31 |
| 11.1 | 47 | 14 | 29 | 14 | 9 | 68 |
| 33.3 | 18 | 13 | 60 | 7 | 7 | 80 |

### Example 34

A sample of cell free tubulin polymerizes and the sample's fluorescence emission increases. Inhibition of tubulin polymerization by a tubulin binding compounds of the present invention was measured by the dose dependence of cell free tubulin fluorescence. The concentration of compound that reduced tubulin fluorescence by 50% compared to untreated tubulin (IC₅₀) are tabulated below in Table 3:

TABLE 3

| Comp. No. | IC₅₀ (μM) |
|---|---|
| 30 | 3.9 |
| 39 | 7.9 |

### Example 35

An in vitro assessment of metabolic stability of compounds was performed using commercially available mouse liver microsomes (MLM) containing cytochrome P450 enzymes (Cedra Corp, Austin, Tex.). A solutions (5 μM) of a compound and microsomes (1 mg/mL protein) was prepared. P450 enzymatic reactions were initiated by adding an NADPH solution. Enzymatic reactions were carried out in a thermostated shaking water bath kept at 37° C. Fifty μl of the reaction mixture was withdrawn immediately and 30 minutes after the addition of the NADPH solution and the proteins were precipitated with acetonitrile. The clear supernatant was analyzed by reversed phase LC-MS/MS (Applied Biosystems API-3000 with Hypersil-BDS C18 column and gradient elution), with internal standard area ratio quantification for the amount the compound remaining as shown in table 4.

### Example 36

For an assessment of plasma stability of compounds, commercially available mouse plasma (Bioreclamation, Hicksville, N.Y.) was added to a DMSO solution of a compound, to a concentration of 5 μM. The reaction mixture (50 μl) was withdrawn immediately and after 30 minutes at 37° C., proteins were precipitated with acetonitrile. The clear supernatant was analyzed by reversed phase LC-MS/MS and the amount the compound remaining quantified as shown in table 4.

TABLE 4

| Compound | Metabolic stability (MLM, | Plasma stability (% |
|---|---|---|
| No. | % remaining at 30 min) | remaining at 30 min) |
| 30 | 101 ± 16 | 104 |
| 35 | 72 ± 9 | 88 |
| 57 | 21 ± 4 | |
| 58 | 28 ± 6 | |
| 60 | 94 ± 4 | |
| 66 | 88 ± 11 | |
| 68 | 17 ± 4 | |
| 70 | 88 ± 12 | 100 |
| 71 | 55 ± 13 | 96 |
| 72 | 40 ± 9 | 105 |

### Example 37

Example 28 describes the usefulness of a compound of this invention in treating cancer as demonstrated employing a H460 xenograft mouse model.

Female CB17/SCID mice (purchased from Taconic, Oxnard, Calif.), 7-8 weeks of age, were allowed to acclimatize for at least three days, and handled under pathogen-free conditions. Human non-small cell lung cancer cell line NCI-H60 was obtained from the American Type Culture Collection. The cell lines were cultured in RPMI 1640 media supplemented with 10% fetal bovine serum. Cells were maintained in a 37° C. incubator with 5% CO₂. The H460 cells were harvested from culture and inoculated at 1×10⁶ cells/animal in the peritoneal subcutaneous space. When the tumors grew to an average volume of 100 mm³ (day 8), each group of mice (ten per group) was administered for five days, vehicle alone (the vehicle group), compound 30 alone at a daily dose of 5, 20, and 50 mg/kg (treatment group), and compound 30 alone at a daily dose of 5, and 20 mg/kg in combination with Taxol® at a daily dose of 10 mg/kg (combination group). Taxol was administered approximately 2-3 hours before that of compound 30.

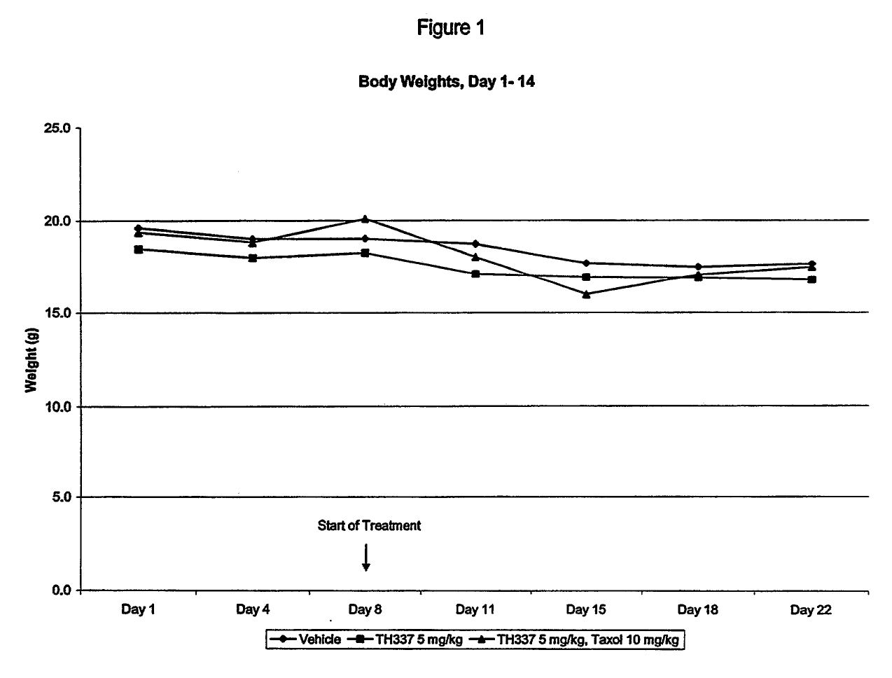

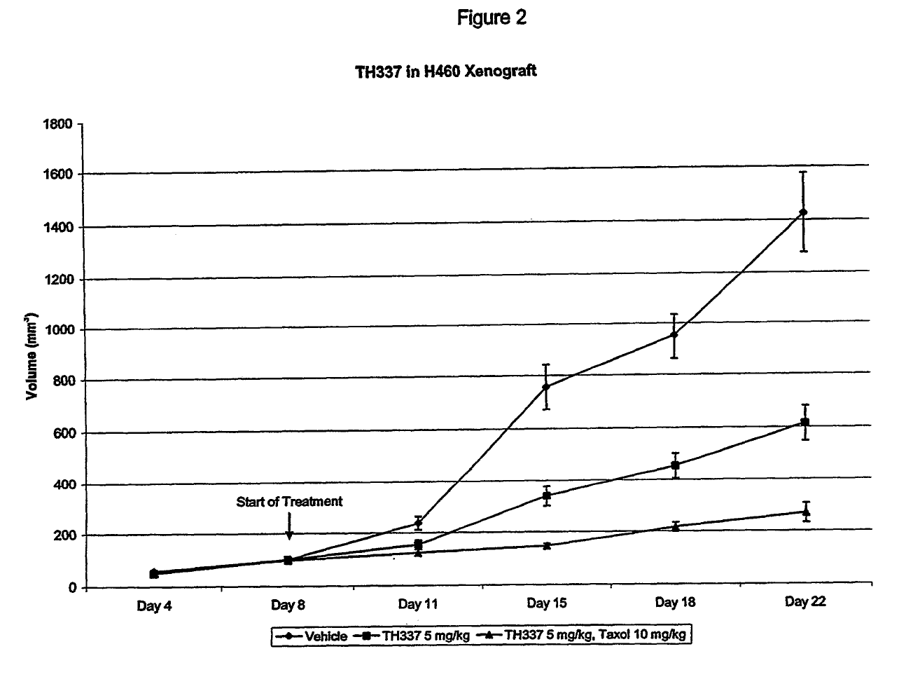

Compound 30, administered at doses greater than 5 mg/kg were toxic and caused lethality in both treatment and combination groups perhaps indicating that the maximum tolerated dose of compound 30 was between 5 and 20 mg/kg. The results from the experiment employing a daily dose compound 30 (5 mg/kg) are shown graphically in FIGS. 1 and 2 below.

The body weight of each mouse was recorded twice per week (FIG. 1). The treatment group administered a daily dose of 5 mg/kg exhibited a weight pattern similar to that of the vehicle group with a mean weight loss of 8% on day 22 from the start of treatment on day 8. Animals in the combination group displayed a weight loss of 13%. One animal in the treatment group was found dead on day 18, and two were found dead on day 22 in the combination group.

FIG. 2 graphically illustrates the mean tumor volume for each treatment group. Growth of each xenograft was monitored by externally measuring tumors in two dimensions using a digital caliper twice per week. Tumor volume (V) was determined by the following equation: V=(L×W²)/2, where L is the length and W is the width of a xenograft. Tumor volumes were measured twice weekly. On day 11, the Treated/Control (T/C) ratio was 65% and 52% in the treatment and combination groups, respectively. At the final measurement (day 22), the T/C ratio for the treatment and combination groups respectively were 43% and 19%.

Employing the same mouse model, when taxol was administered alone in the same dose and schedule as used for the combination group above, and when treatment began at 150 mm³ volume of xenograft tumors, the TIC for day 21 was 56%. The xenograft data for compound 30 demonstrates that compared to the known anticancer agent taxol, compound 30 can show in vivo anti tumor activity both as a single agent and in combination with taxol.

Although the present invention has been described in detail with reference to specific embodiments, those of skill in the art will recognize that modifications and improvements are within the scope and spirit of the invention, as set forth in the claims which follow. All publications and patent documents (patents, published patent applications, and unpublished patent applications) cited herein are incorporated herein by reference as if each such publication or document was specifically and individually indicated to be incorporated herein by reference. Citation of publications and patent documents is not intended as an admission that any such document is pertinent prior art, nor does it constitute any admission as to the contents or date of the same. The invention having now been described by way of written description and example, those of skill in the art will recognize that the invention can be practiced in a variety of embodiments and that the foregoing description and examples are for purposes of illustration and not limitation of the following claims.

We claim:

1. A compound selected from: 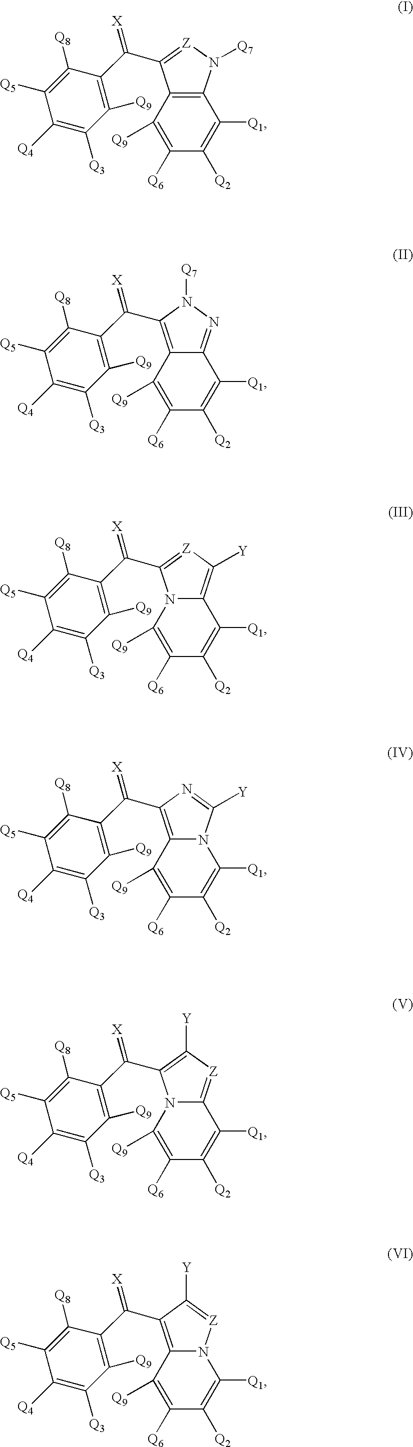 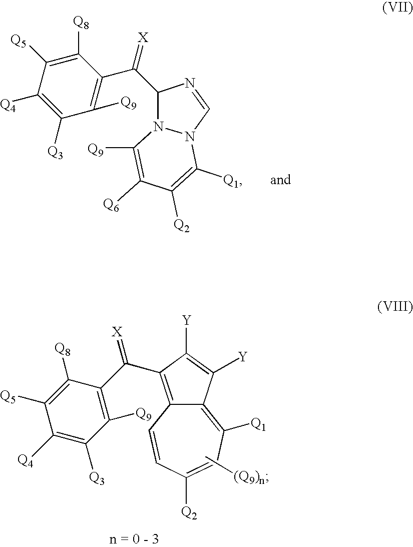
wherein each Q₁, Q₂, and Q₆ independently is hydrogen; halo; amino; C₁-C₆ alkylamino; di C₁-C₆ alkylamino; hydroxyl; C₁-C₆ alkoxy; nitro; cyano; C₁-C₆ alkyl; C₁-C₆ heteroalkyl; C₂-C₆ alkenyl; C₂-C₆ alkynyl; C₃-C₈ cycloalkyl; C₃-C₈ heterocyclyl; aryl; heteroaryl; COR₁₈; SO₂R₁₈; or PO₃R₁₈;
each Q₃-Q₅ is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈ or PO₃R₁₈; Q₃ and Q₄ together form C₃-C₈ heterocycle, an aryl, or a heteroaryl; or Q₄ and Q₅ together form a C₃-C₈ heterocycle, an aryl, or a heteroaryl; with the proviso that in any one compound, only one of Q₃-Q₅ is hydrogen;
Q₇ is hydrogen; amino; C₁-C₆ alkylamino; di C₁-C₆ alkylamino; hydroxyl; C₁-C₆ alkoxy; nitro; cyano; C₁-C₆ alkyl; C₁-C₆ heteroalkyl; C₂-C₆ alkenyl; C₂-C₆ alkynyl; C₃-C₈ cycloalkyl; C₃-C₈ heterocyclyl; aryl; heteroaryl; COR₁₅; SO₂R₁₈; PO₃R₁₈ or a monosaccharide; with the proviso that in formula (II) Q₇ excludes hydrogen;
Q₈ is hydrogen; halo; amino; C₁-C₆ alkylamino; di C₁-C₆ alkylamino; hydroxyl; C₁-C₆ alkoxy; nitro; cyano; aryl; heteroaryl; COR₁₅; SO₂R₁₅ or PO₃R₁₅;
each Q₉ independently is hydrogen; halo; amino; C₁-C₆ alkylamino; di C₁-C₆ alkylamino; hydroxyl; C₁-C₆ alkoxy; nitro; cyano; aryl; heteroaryl; COR₁₅; SO₂R₁₅ or PO₃R₁₅;
X is O, —NNHR₁₆, NR₁₆, or NOR₁₆;
Y is hydrogen, hydroxyl, or halogen;
Z is —CH— or —N—;
R₁₅ is hydrogen, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl;
R₁₆ is hydrogen, C₁-C₆ alkyl, aryl, C₁-C₆ alkylsulphonyl, arylsulfonyl, C₁-C₆alkoxycarbonyl, aminocarbonyl, C₁-C₆alkylaminocarbonyl, di C₁-C₆ alkylaminocarbonyl, C₁-C₆ acyl, aroyl, aminothiocarbonyl, C₁-C₆ alkylaminothiocarbonyl, di C₁-C₆ alkylaminothiocarbonyl, C₁-C₆ thioacyl, or thioaroyl; with the proviso that when X is NR₁₆, R₁₆ excludes hydrogen;
R₁₈ is hydrogen, hydroxyl, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl; or
a tautomer or an individual isomer or a racemic or non-racemic mixture of isomers, a polymorph, a hydrate or a pharmaceutically acceptable salt or solvate thereof.

2. The compound of claim 1, wherein
Q₁ is hydrogen; halo; cyano; nitro; COR₁₅; SO₂R₁₅; PO₃R₁₅; ≡R₁₃ or 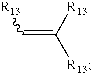
Q₂ is ≡R₁₃; 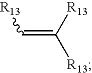
C₁-C₆ alkoxy; halo; amino; or hydroxy;
each Q₃, Q₄ and Q₅ independently is hydrogen, C₁-C₆ alkoxy, halo, amino, hydroxyl, Q₃ and Q₄ together is methylenedioxy, or Q₄ and Q₅ together is methylenedioxy, provided that in any compound only one of the Q₃, Q₄ and Q₅ is hydrogen;
Q₇ is C₁-C₆ alkyl optionally substituted independently with one or more aryl, heteroaryl, hydroxyl, amino, C₁-C₆alkylamino, di C₁-C₆alkylamino, CO₂H, or CONH₂; COR₁₅; SO₂R₁₈; or PO₃R₁₈; or a monosaccharide;
R₁₃ is hydrogen; C₁-C₆ alkyl, C₁-C₆ heteroalkyl, C₃-C₈ cycloalkyl, C₃-C₈ heterocyclyl, aryl, or heteroalkyl each optionally substituted with hydroxyl, C₁-C₆ alkoxy, amino, C₁-C₆ alkylamino, di C₁-C₆ alkylamino, NHCOR₁₅, or COR₁₈; and
R₁₈ is hydrogen, hydroxyl, C₁-C₆ alkoxy, amino, C₁-C₆alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl.

3. The compound of claim 2, wherein
Q₁ is hydrogen; halo; cyano; CO₂H; CONH₂; ≡R₁₃; or 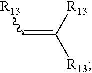
and
each Q₂-Q₆ independently is hydrogen, C₁-C₆, alkoxy; halo; amino; or hydroxy; with the proviso that in any compound only one of the Q₃, Q₄, and Q₅ is hydrogen.

4. The compound of claim 3, selected from formulas (I-i), (III-i), (IV-i), (V-i), (VI-i), (VII-i) and (VIII-i): 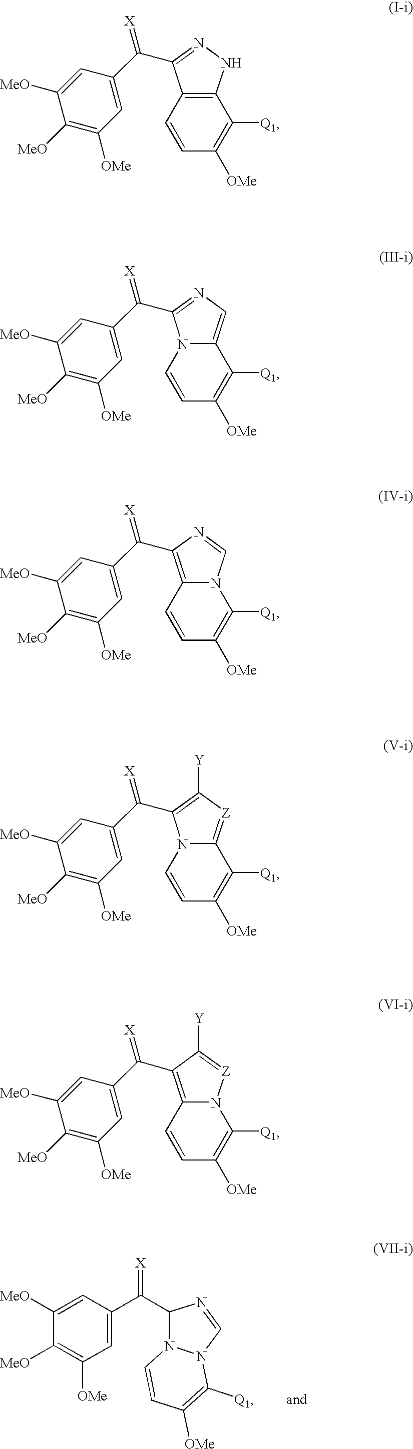 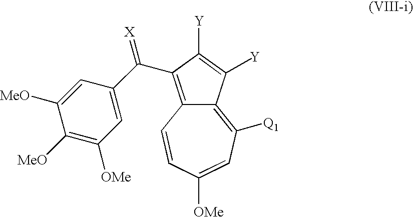
wherein Q₁ is 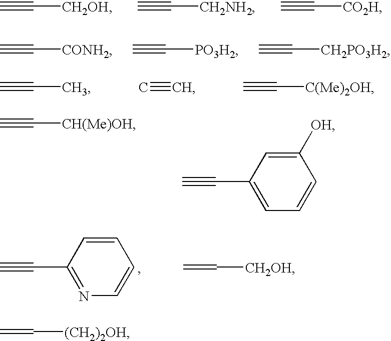
—CH₂—CH₂—OH, —CH₂—CH₂—CH₂—OH, —CONH₂, —CO₂H, —CN, or halo.

5. The compound of claim 4 wherein X is O.

6. The compound of claim 5 of formula:

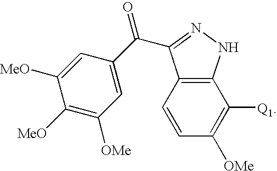

7. The compound of claim 6 wherein Q1 is

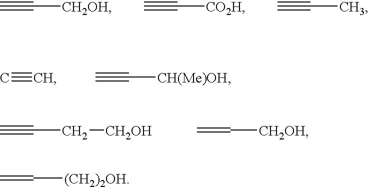

8. A compound of formula (XIV): 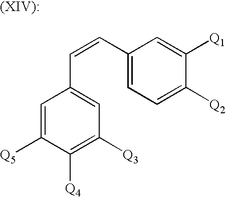
wherein
Q₁ is ≡R₁₃ or 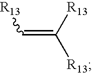
Q₂ is ≡R₁₃; 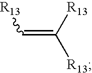
C₁-C₆ alkoxy; halo; amino; or hydroxy;
each Q₃-Q₅ is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈; or PO₃R₁₈; Q₃ and Q₄ together form C₃-C₈ heterocycle, an aryl, or a heteroaryl; or Q₄ and Q₅ together form a C₃-C₈ heterocycle, an aryl, or a heteroaryl; with the proviso that in any one compound, only one of Q₃-Q₅ is hydrogen;
R₁₃ is hydrogen; C₁-C₆ alkyl, C₁-C₆ heteroalkyl, C₃-C₈ cycloalkyl, C₃-C₈ heterocyclyl, aryl, or heteroalkyl each optionally substituted with hydroxyl, C₁-C₆ alkoxy, amino, C₁-C₆ alkylamino, di C₁-C₆ alkylamino; COR₁₈ or NHCOR₁₅;
R₁₅ is hydrogen, hydroxyl, C₁-C₆ alkoxy, amino, C₁-C₆alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl; or
a tautomer or an individual isomer or a racemic or non-racemic mixture of isomers, a polymorph, a hydrate or a pharmaceutically acceptable salt or solvate thereof.

9. The compound of claim 8, wherein
Q₁ is ≡R₁₃; or 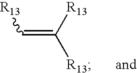
each Q₂-Q₅ independently is hydrogen, C₁-C₆ alkoxy; halo; amino; hydroxy; Q₃ and Q₄ together is methylenedioxy; or Q₄ and Q₅ together is methylenedioxy; with the proviso that in any compound only one of the Q₃, Q₄ and Q₅ is hydrogen.

10. The compound of claim 9 of formula:
the compound of formula: 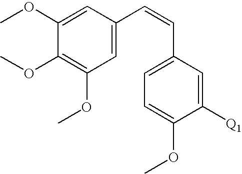
wherein Q₁ is

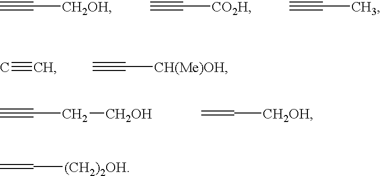

11. A compound of formula (XV): 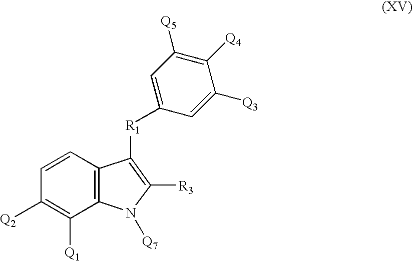
wherein
Q₁ is ≡R₁₃ or 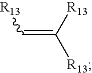
Q₂ is ≡R₁₃ 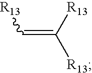
C₁-C₆ alkoxy; halo; amino; or hydroxy;
each Q₃, Q₄, and Q₅ independently is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈; or PO₃R₁₈; Q₃ and Q₄ together form C₃-C₈ heterocycle, an aryl, or a heteroaryl; or Q₄ and Q₅ together form a C₃-C₈ heterocycle, an aryl, or a heteroaryl; with the proviso that in any one compound, only one of Q₃-Q₅ is hydrogen;
Q₇ is hydrogen; amino; C₁-C₆ alkylamino; di C₁-C₆ alkylamino; hydroxyl; C₁-C₆ alkoxy; nitro; cyano; C₁-C₆ alkyl; C₁-C₆ heteroalkyl; C₁-C₆ alkenyl; C₁-C₆ alkynyl; C₃-C₈ cycloalkyl; C₃-C₈ heterocyclyl; aryl; heteroaryl; COR₁₅; SO₂R₁₈; or PO₃R₁₈ or a monosaccharide;
R₁ is CH₂ or CO;
R₃ is hydrogen, halo, C₁-C₆ alkyl, aryl or heteroaryl;
R₁₃ is hydrogen; C₁-C₆ alkyl, C₁-C₆ heteroalkyl, C₃-C₈ cycloalkyl, C₃-C₈ heterocyclyl, aryl, or heteroalkyl each optionally substituted with hydroxyl, C₁-C₆ alkoxy, amino, C₁-C₆ alkylamino, di C₁-C₆ alkylamino; NHCOR₁₅ or COR₁₈
R₁₅ is hydrogen, hydroxyl, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₁-C₆ alkenyl, C₁-C₆ alkynyl, C₃-C₆ cycloalkyl, C₃-C₆ heterocyclyl, aryl, or heteroaryl;
R₁₈ is R₁₈ is hydrogen, hydroxyl, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl; or
a tautomer or an individual isomer or a racemic or non-racemic mixture of isomers, a polymorph, a hydrate or a pharmaceutically acceptable salt or solvate thereof.

12. The compound of claim 11, wherein
each Q₂-Q₅ independently is hydrogen, C₁-C₆, alkoxy; halo; amino; or hydroxy; with the proviso that in any compound only one of the Q₃, Q₄ and Q₅ is hydrogen.

13. A compound of claim 11 of the formulas (XV-i), (XV-ii) and (XV-iii) 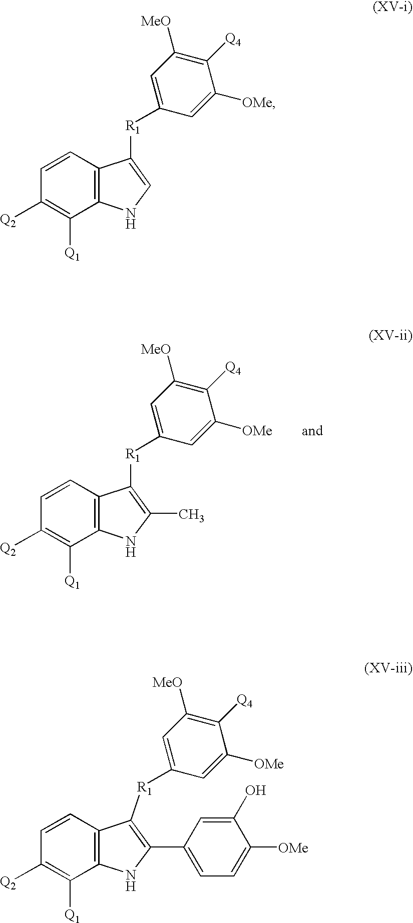
wherein Q₂ is C₁-C₆ alkoxy; and Q₄ is hydrogen or methoxy; or a tautomer or an individual isomer or a racemic or non-racemic mixture of isomers, a polymorph, a hydrate or a pharmaceutically acceptable salt or solvate thereof.

14. A compound selected from formulas (XVI)-(XX): 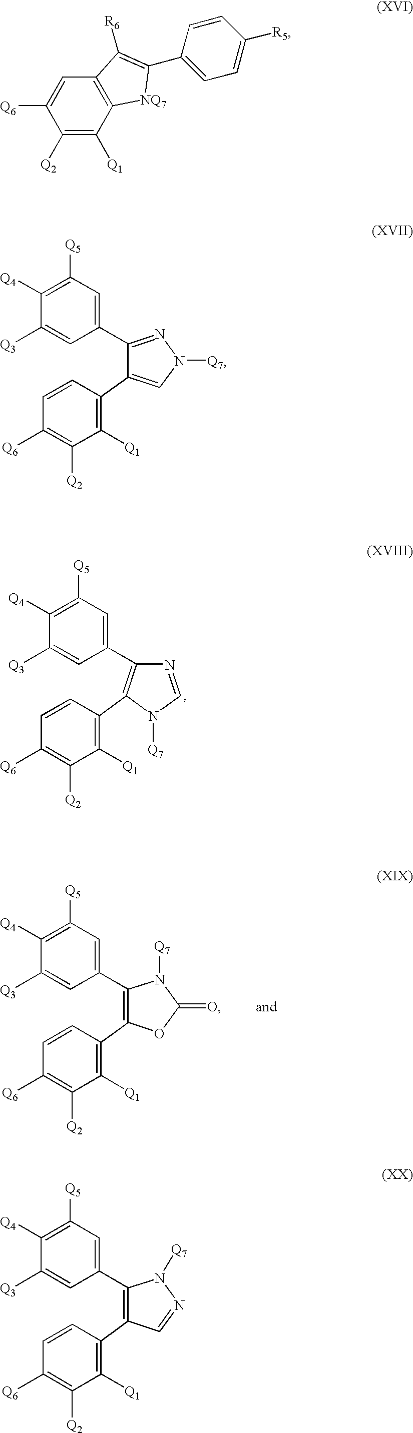
wherein
Q₁ is ≡R₁₃ or 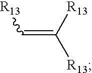
Q₂ is ≡R₁₃; 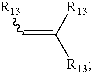
C₁-C₆ alkoxy; halo; amino; or hydroxy;
each Q₃-Q₅ is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈; or PO₃R₁₈; Q₃ and Q₄ together form C₃-C₈ heterocycle, an aryl, or a heteroaryl; or Q₄ and Q₅ together form a C₃-C₈ heterocycle, an aryl, or a heteroaryl; with the proviso that in any one compound, only one of Q₃-Q₅ is hydrogen;
Q₆ is hydrogen; halo; amino; C₁-C₆ alkylamino; di C₁-C₆alkylamino; hydroxyl; C₁-C₆ alkoxy; nitro; cyano; C₁-C₆ alkyl; C₁-C₆ heteroalkyl; C₁-C₆ alkenyl; C₁-C₆ alkynyl; C₃-C₈ cycloalkyl; C₃-C₈ heterocyclyl; aryl; heteroaryl; COR₁₈; SO₂R₁₈ or PO₃R₁₈;
Q₇ is hydrogen; amino; C₁-C₆ alkylamino; di C₁-C₆ alkylamino; hydroxyl; C₁-C₆ alkoxy; nitro; cyano; C₁-C₆ alkyl; C₁-C₆ heteroalkyl; C₁-C₆ alkenyl; C₁-C₆ alkynyl; C₃-C₈ cycloalkyl; C₃-C₈ heterocyclyl; aryl; heteroaryl; COR₁₅; SO₂R₁₈; or PO₃R₁₈; or a monosaccharide;
R₅ is hydrogen, halo, or C₁-C₆ alkoxy;
R₆ is formyl or a protected form thereof;
R₁₃ is hydrogen; C₁-C₆ alkyl, C₁-C₆ heteroalkyl, C₃-C₈ cycloalkyl, C₃-C₈ heterocyclyl, aryl, or heteroalkyl each optionally substituted with hydroxyl, C₁-C₆ alkoxy, amino, C₁-C₆ alkylamino, di C₁-C₆ alkylamino, NHCOR₁₅ or COR₁₈;
R₁₅ is hydrogen, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl;
R₁₈ is hydrogen, hydroxyl, C₁-C₆ alkoxy, amino, C₁-C₆alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl; or
a tautomer or an individual isomer or a racemic or non-racemic mixture of isomers, a polymorph, a hydrate or a pharmaceutically acceptable salt or solvate thereof.

15. The compound of claim 14, wherein
each Q₂ and Q₆ independently is hydrogen, hydroxy, C₁-C₆ alkoxy, halo, or amino; and each Q₃, Q₄, and Q₅ is OMe; Q₃ and Q₄ together is methylenedioxy, or Q₄ and Q₅ together is methylenedioxy.

16. The compound of claim 15 wherein Q₂ is hydrogen, hydroxyl, fluoro or methoxy; Q₆ is hydrogen, hydroxyl, fluoro, methoxy or amino.

17. A compound of formulas (XXI)-(XXVII): 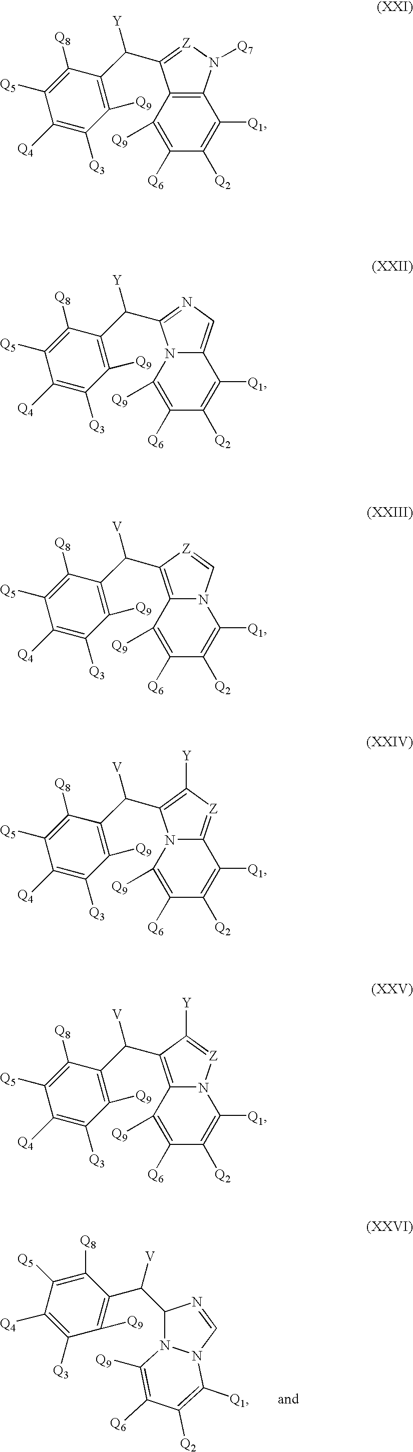 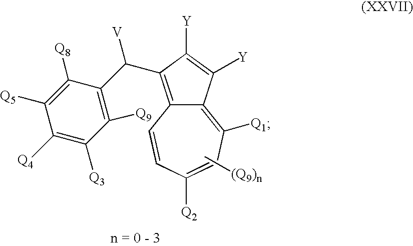
wherein each Q₁, Q₂, and Q₆ independently is hydrogen; halo; amino; C₁-C₆ alkylamino; di C₁-C₆ alkylamino; hydroxyl; C₁-C₆ alkoxy; nitro; cyano; C₁-C₆ alkyl; C₁-C₆ heteroalkyl; C₁-C₆ alkenyl; C₁-C₆ alkynyl; C₃-C₈ cycloalkyl; C₃-C₈ heterocyclyl; aryl; heteroaryl; COR₁₈; SO₂R₁₈ or PO₃R₁₈;
each Q₃-Q₅ is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈; or PO₃R₁₈; Q₃ and Q₄ together form C₃-C₈ heterocycle, an aryl, or a heteroaryl; or Q₄ and Q₅ together form a C₃-C₈ heterocycle, an aryl, or a heteroaryl; with the proviso that in any one compound, only one of Q₃-Q₅ is hydrogen;
Q₇ is hydrogen; halo; amino; C₁-C₆ alkylamino; di C₁-C₆ alkylamino; hydroxyl; C₁-C₆ alkoxy; nitro; cyano; C₁-C₆ alkyl; C₁-C₆ heteroalkyl; C₁-C₆ alkenyl; C₁-C₆ alkynyl; C₃-C₈ cycloalkyl; C₃-C₈ heterocyclyl; aryl; heteroaryl; COR₁₅; SO₂R₁₈; or PO₃R₁₈; or a monosaccharide; with the proviso that in formula (II) Q₇ excludes hydrogen;
Q₈ is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈ or PO₃R₁₈;
each Q₉ independently is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈ or PO₃R₁₈;
V is —NHR₁₆; —NHNHR₁₆; —NHN(R₁₆)₂; —NR₁₆NHR₁₆; or —OR₁₇;
Y is hydrogen, hydroxyl or halogen;
Z is —CH— or —N—;
R₁₅ is hydrogen, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₁-C₆ alkenyl, C₁-C₆ alkynyl, C₁-C₆ cycloalkyl, C₁-C₆ heterocyclyl, aryl, or heteroaryl;
R₁₆ is hydrogen, C₁-C₆ alkyl, aryl, C₁-C₆ alkylsulphonyl, arylsulfonyl, C₁-C₆ alkoxycarbonyl, aminocarbonyl, C₁-C₆ alkylaminocarbonyl, di C₁-C₆ alkylaminocarbonyl, C₁-C₆ acyl, aroyl, aminothiocarbonyl, C₁-C₆ alkylaminothiocarbonyl, di C₁-C₆ alkylaminothiocarbonyl, C₁-C₆ thioacyl, or thioaroyl; and R′ is C₁-C₆ alkyl or aryl; with the proviso that when V is NR₁₆, R₁₆ excludes hydrogen;
R₁₇ is C₁-C₆ alkyl; aryl; or di C₁-C₆ alkylamino;
R₁₈ is hydrogen, hydroxyl, C₁-C₆ alkoxy, amino, C₁-C₆ alkylamino, di C₁-C₆alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl; or
a tautomer or an individual isomer or a racemic or non-racemic mixture of isomers, a polymorph, a hydrate or a pharmaceutically acceptable salt or solvate thereof.

18. The compound of claim 17, wherein
Q₁ is hydrogen; halo; cyano; nitro; COR₁₈; SO₂R₁₈; PO₃R₁₈; ≡R₁₃ or 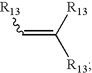
Q₂ is ≡R₁₃; 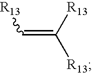
C₁-C₆ alkoxy; halo; amino; or hydroxy;
each Q₃, Q₄ and Q₅ independently is hydrogen, C₁-C₆ alkoxy, halo, amino, or hydroxyl provided that in any compound only one of the Q₃, Q₄ and Q₅ is hydrogen; Q₃ and Q₄ together is methylenedioxy; or Q₄ and Q₅ together is methylenedioxy;
Q₇ is C₁-C₆ alkyl optionally substituted independently with one or more aryl, heteroaryl, hydroxyl, amino, C₁-C₆alkylamino, di C₁-C₆alkylamino, CO₂H, or CONH₂; COR₁₅; SO₂R₁₈; or PO₃R₁₈; or a monosaccharide;
R₁₃ is hydrogen; C₁-C₆ alkyl, C₁-C₆ heteroalkyl, C₃-C₈ cycloalkyl, C₃-C₈ heterocyclyl, aryl, or heteroalkyl each optionally substituted with hydroxyl, C₁-C₆ alkoxy, amino, C₁-C₆ alkylamino, di C₁-C₆ alkylamino; NHCOR₁₅ or COR₁₈
R₁₅ is hydrogen, hydroxyl, C₁-C₆ alkoxy, amino, C₁-C₆ alkylamino, di C₁-C₆alkylamino, NHOH, NHNH₂, C₁-C₆ alkyl, C₁-C₆ heteroalkyl, C₁-C₆ alkenyl, C₁-C₆ alkynyl, C₃-C₆ cycloalkyl, C₃-C₆ heterocyclyl, aryl, or heteroaryl;
R₁₆ is hydrogen, C₁-C₆ alkoxy, amino, C₁-C₆ alkylamino, di C₁-C₆ alkylamino, NHOH, or NHNH₂, C₁-C₆ alkyl, C₁-C₆ heteroalkyl, C₃-C₈ cycloalkyl, C₃-C₈ heterocyclyl, aryl, or heteroalkyl, NHOH, NHNH₂, and
R₁₈ is hydrogen, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl.

19. The compound of claim 18 selected from of formulas (XXI-i), (XXII-i), (XXIII-i), (XXIV-i), (XXV-i) and (XXVII-i):

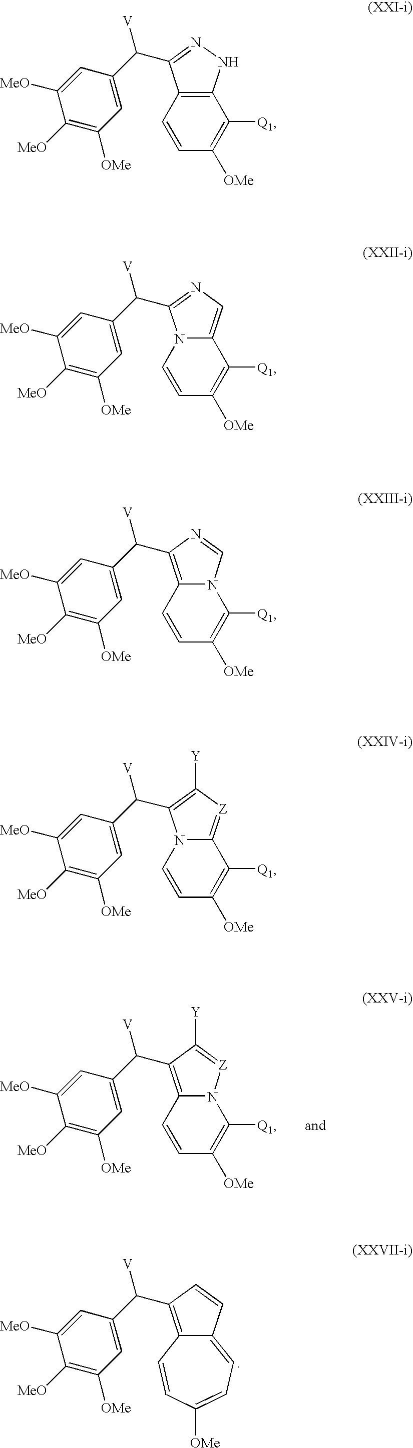

20. A compound of formulas (XXVIII)-(XXXII): 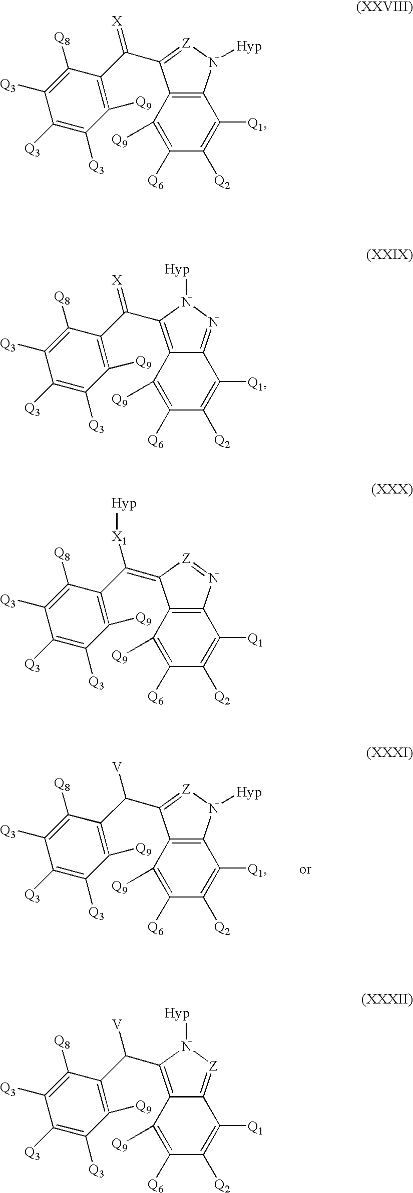
wherein each Q₁, Q₂, and Q₆ independently is hydrogen; halo; amino; C₁-C₆ alkylamino; di C₁-C₆ alkylamino; hydroxyl; C₁-C₆ alkoxy; nitro; cyano; C₁-C₆ alkyl; C₁-C₆ heteroalkyl; C₂-C₆ alkenyl; C₂-C₆ alkynyl; C₃-C₈ cycloalkyl; C₃-C₈ heterocyclyl; aryl; heteroaryl; COR₁₈; SO₂R₁₈; or PO₃R₁₈;
each Q₃-Q₅ is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈ or PO₃R₁₈; Q₃ and Q₄ together form C₃-C₈ heterocycle, an aryl, or a heteroaryl; or Q₄ and Q₅ together form a C₃-C₈ heterocycle, an aryl, or a heteroaryl; with the proviso that in any one compound, only one of Q₃-Q₅ is hydrogen;
Q₈ is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈; or PO₃R₁₅;
each Q₉ independently is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈; or PO₃R₁₈;
V is —NHNHR₁₆; —HNR₁₆; —N(Hyp)NHR₁₆; —NHN(Hyp)R₁₆; or —N(Hyp)N(Hyp)R; wherein Hyp is a hypoxic activator;
X is O, —NNHR₁₆, NR₁₆, —NN(Hyp)R₁₆, or NOR₁₆ wherein R₁₆ is C₁-C₆ alkyl, aryl, C₁-C₆alkylsulphonyl, arylsulfonyl, C₁-C₆alkoxycarbony, aminocarbonyl, C₁-C₆ alkylaminocarbonyl, di C₁-C₆ alkylaminocarbonyl, C₁-C₆ acyl, aroyl, aminothiocarbonyl, C₁-C₆ alkylaminothiocarbonyl, di C₁-C₆ alkylaminothiocarbonyl, C₁-C₆ thioacyl, or thioaroyl; with the proviso that when X is NR₁₆, R₁₆ excludes hydrogen;
X₁ is O;
Y is hydrogen, hydroxyl, or halogen;
Z is —CH— or —N—;
R₁₅ is hydrogen, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl;
R₁₆ is hydrogen, C₁-C₆ alkyl, aryl, C₁-C₆ alkylsulphonyl, arylsulfonyl, C₁-C₆ alkoxycarbonyl, aminocarbonyl, C₁-C₆ alkylaminocarbonyl, di C₁-C₆ alkylaminocarbonyl, C₁-C₆ acyl, aroyl, aminothiocarbonyl, C₁-C₆ alkylaminothiocarbonyl, di C₁-C₆ alkylaminothiocarbonyl, C₁-C₆ thioacyl, or thioaroyl; with the proviso that when X is NR₁₆, R₁₆ excludes hydrogen;
R₁₈ is hydrogen, hydroxyl, C₁-C₆ alkoxy, amino, C₁-C₆alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl; or
a tautomer or an individual isomer or a racemic or non-racemic mixture of isomers, a polymorph, a hydrate or a pharmaceutically acceptable salt or solvate thereof.

21. The compound of claim 20 wherein
Q₁ is hydrogen; halo; cyano; nitro; COR₁₈; SO₂R₁₈; PO₃R₁₈; ≡R₁₃ or 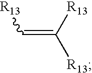
Q₂ is ≡R₁₃: 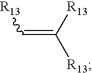
C₁-C₆ alkoxy; halo; amino; or hydroxy;
each Q₃, Q₄ and Q₅ independently is hydrogen, C₁-C₆ alkoxy, halo, amino, hydroxyl, Q₃ and Q₄ together is methylenedioxy, or Q₄ and Q₅ together is methylenedioxy, provided that in any compound only one of the Q₃, Q₄, and Q₅ is hydrogen;
R₁₃ is hydrogen; C₁-C₆ alkyl, C₁-C₆ heteroalkyl, C₃-C₈ cycloalkyl, C₃-C₈ heterocyclyl, aryl, or heteroalkyl each optionally substituted with hydroxyl, C₁-C₆ alkoxy, amino, C₁-C₆ alkylamino, di C₁-C₆ alkylamino; NHCOR₁₅ or COR₁₈

22. The compound of claim 21 having formula: 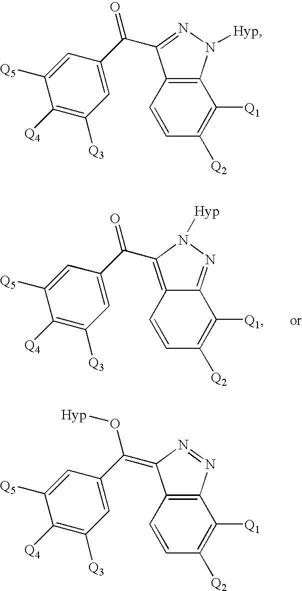
wherein Q₁ is hydrogen, 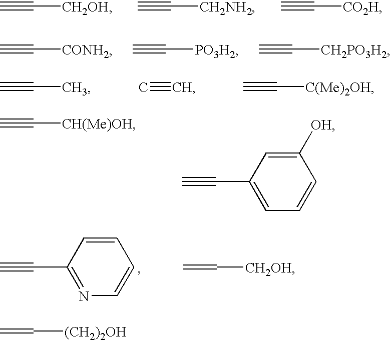
—CH₂—CH₂—OH, —CH₂—CH₂—CH₂—OH, —CONH₂, —CO₂H, —CN, or halo.

23. The compound of claim 22 selected from the group consisting of

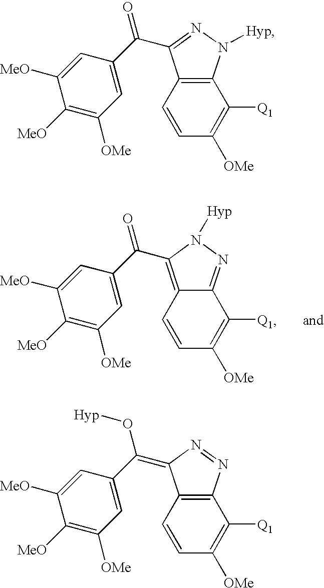

24. A compound of claim 23 of formula:

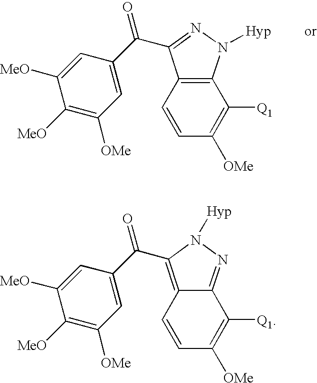

25. A compound of formula (XIV): 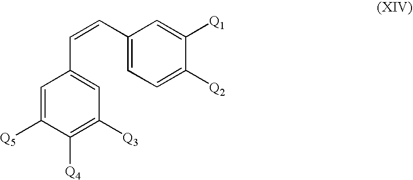
wherein
Q₁ is ≡R₁₃ or 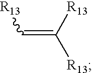
Q₂ is ≡R₁₃ 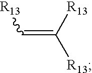
C₁-C₆ alkoxy; halo; amino; or hydroxy;
each Q₃-Q₅ is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈, or PO₃R₁₈; Q₃ and Q₄ together form C₃-C₈ heterocycle, an aryl, or a heteroaryl; or Q₄ and Q₅ together form a C₃-C₈ heterocycle, an aryl, or a heteroaryl; (-OHyp) or (-NHyp) with the proviso that in any one compound, at least one of Q₃-Q₅ is (-OHyp) or (-NHyp);
R₁₃ is hydrogen; C₁-C₆ alkyl, C₁-C₆ heteroalkyl, C₃-C₈ cycloalkyl, C₃-C₈ heterocyclyl, aryl, or heteroalkyl each optionally substituted with hydroxyl, C₁-C₆ alkoxy, amino, C₁-C₆ alkylamino, di C₁-C₆ alkylamino; COR₁₈ or NHCOR₁₅;
R₁₅ is hydrogen, hydroxyl, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl;
Hyp is hypoxic activator; or
a tautomer or an individual isomer or a racemic or non-racemic mixture of isomers, a polymorph, a hydrate or a pharmaceutically acceptable salt or solvate thereof.

26. The compound of claim 25 wherein Q₁ is ≡R₁₃; or 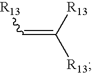
and each Q₂-Q₅ independently is hydrogen, C₁-C₆ alkoxy; halo; amino; or hydroxy; with the proviso that in any compound at least one of Q₃-Q₅ is (-OHyp) or (-NHyp); or a tautomer or an individual isomer or a racemic or non-racemic mixture of isomers, a polymorph, a hydrate or a pharmaceutically acceptable salt or solvate thereof.

27. A compound of formula (XXXIV): 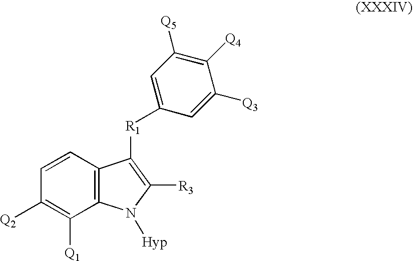
wherein
Q₁ is ≡R₁₃ or 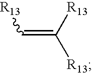
Q₂ is ≡R₁₃; 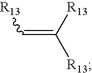
C₁-C₆ alkoxy; halo; amino; or hydroxy;
each Q₃, Q₄, and Q₅ independently is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈; or PO₃R₁₈; Q₃ and Q₄ together form C₃-C₈ heterocycle, an aryl, or a heteroaryl; or Q₄ and Q₅ together form a C₃-C₈ heterocycle, an aryl, or a heteroaryl;
with the proviso that in any one compound, only one of Q₃-Q₅ is hydrogen;
R₁ is CH₂ or CO;
R₃ is hydrogen, halo, C₁-C₆ alkyl, aryl, or heteroaryl;
R₁₃ is hydrogen; C₁-C₆ alkyl, C₁-C₆ heteroalkyl, C₃-C₈ cycloalkyl, C₃-C₈ heterocyclyl, aryl, or heteroalkyl each optionally substituted with hydroxyl, C₁-C₆ alkoxy, amino, C₁-C₆ alkylamino, di C₁-C₆ alkylamino, NHCOR₁₅, or COR₁₈;
R₁₅ is hydrogen, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₁-C₆ alkenyl, C₁-C₆ alkynyl, C₃-C₆ cycloalkyl, C₃-C₆ heterocyclyl, aryl, or heteroaryl;
R₁₈ is hydrogen, hydroxyl, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl;
Hyp is hypoxic activator; or
a tautomer or an individual isomer or a racemic or non-racemic mixture of isomers, a polymorph, a hydrate or a pharmaceutically acceptable salt or solvate thereof.

28. The compound of claim 27 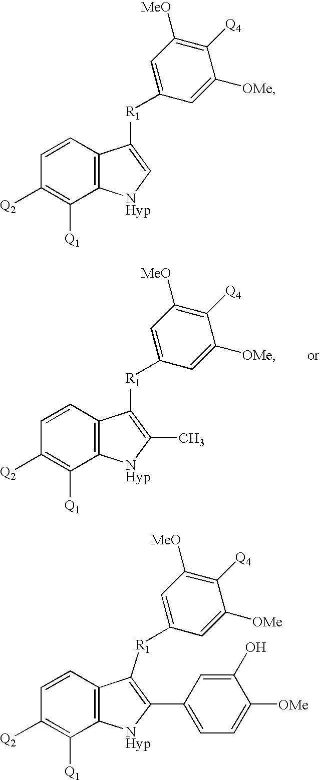
wherein Q₂ is C₁-C₆ alkoxy and Q₄ is hydrogen or methoxy.

29. A compound selected from formulas (XXXV)-(XXXIX): 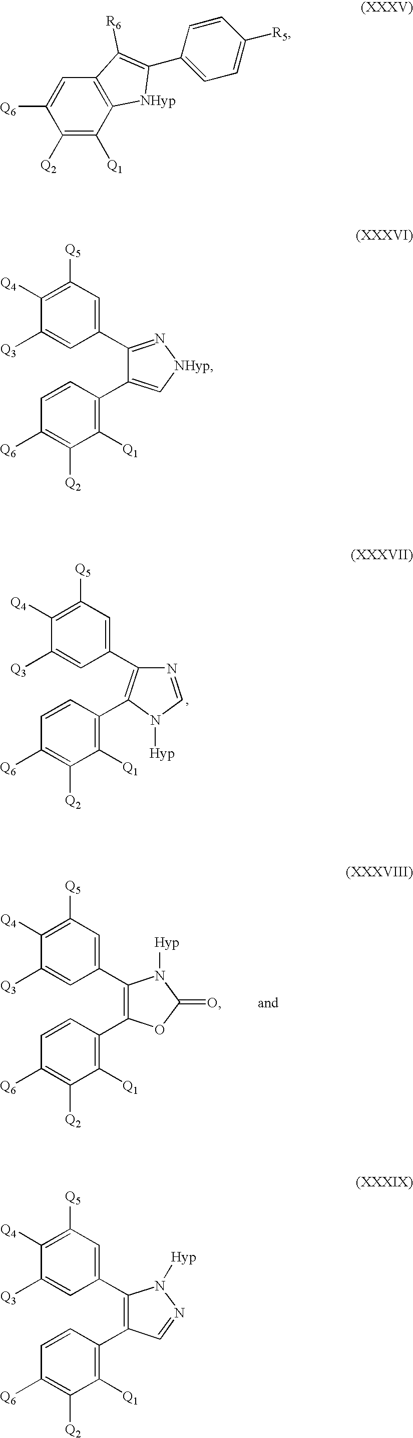
wherein
Q₁ is ≡R₁₃ or 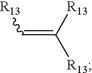
Q₂ is ≡R₁₃; 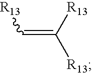
C₁-C₆ alkoxy; halo; amino; or hydroxy;
each Q₃-Q₅ is hydrogen; halo; amino; C₁-C₆ alkylamino; di C₁-C₆ alkylamino; hydroxyl; C₁-C₆ alkoxy; nitro; cyano; heteroaryl; COR₁₈; SO₂R₁₈; or PO₃R₁₈ with the proviso that in any one compound, only one of Q₃-Q₅ is hydrogen; Q₃ and Q₄ together form C₃-C₈ heterocycle, an aryl, or a heteroaryl; or Q₄ and Q₅ together form a C₃-C₈ heterocycle, an aryl, or a heteroaryl;
Q₆ is hydrogen; halo; amino; C₁-C₆ alkylamino; di C₁-C₆ alkylamino;
hydroxyl; C₁-C₆ alkoxy; nitro; cyano; C₁-C₆ alkyl; C₁-C₆ heteroalkyl; C₁-C₆ alkenyl; C₁-C₆ alkynyl; C₃-C₈ cycloalkyl; C₃-C₈ heterocyclyl; aryl; heteroaryl; COR₁₅; SO₂R₁₅ or PO₃R₁₅;
Q₇ is hydrogen; amino; C₁-C₆ alkylamino; di C₁-C₆ alkylamino; hydroxyl; C₁-C₆ alkoxy; nitro; cyano; C₁-C₆ alkyl; C₁-C₆ heteroalkyl; C₁-C₆ alkenyl; C₁-C₆ alkynyl; C₃-C₈ cycloalkyl; C₃-C₈ heterocyclyl; aryl; heteroaryl; COR₁₈; SO₂R₁₅; or PO₃R₁₅; or a monosaccharide;
R₅ is hydrogen, halo, or C₁-C₆ alkoxy;
R₆ is formyl or a protected form thereof;
R₁₃ is hydrogen; C₁-C₆ alkyl, C₁-C₆ heteroalkyl, C₃-C₈ cycloalkyl, C₃-C₈ heterocyclyl, aryl, or heteroalkyl each optionally substituted with hydroxyl, C₁-C₆ alkoxy, amino, C₁-C₆ alkylamino, di C₁-C₆ alkylamino, NHCOR₁₅ or COR₁₅;
R₁₅ is hydrogen, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl;
R₁₈ is hydrogen, hydroxyl, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl;
Hyp is hypoxic activator; or
a tautomer or an individual isomer or a racemic or non-racemic mixture of isomers, a polymorph, a hydrate or a pharmaceutically acceptable salt or solvate thereof.

30. The compound of claim 29, wherein
each Q₂ and Q₆ independently is hydrogen, hydroxy, C₁-C₆ alkoxy, halo, or amino; and each Q₃, Q₄, and Q₅ is OMe.

31. The compound of claim 29 wherein Q₂ is hydrogen, hydroxyl, fluoro or methoxy; Q₆ is hydrogen, hydroxyl, fluoro, methoxy or amino.

32. A compound selected from formulas (XL)-(XLIII) 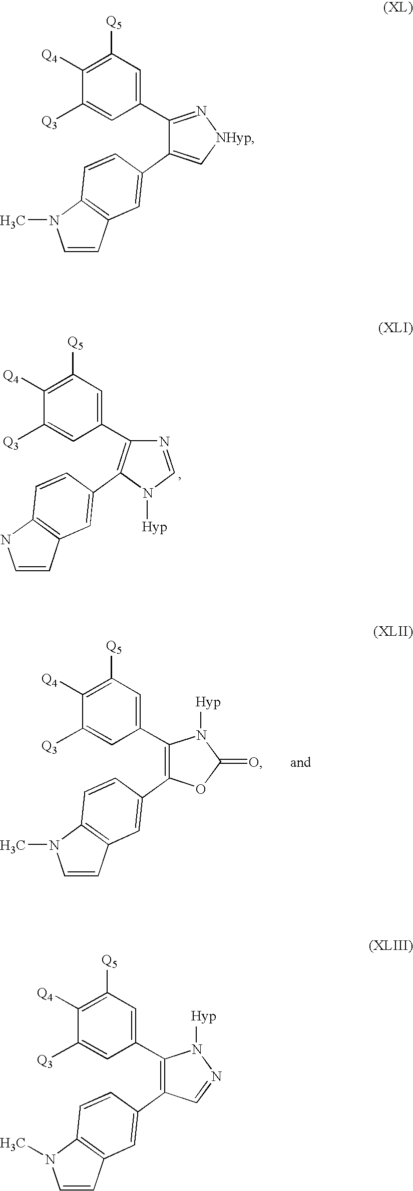
wherein
each Q₃-Q₅ is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈; or PO₃R₁₈ with the proviso that in any one compound, only one of Q₃-Q₅ is hydrogen;
R₁₅ is hydrogen, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl;
R₁₈ is hydrogen, hydroxyl, C₁-C₆ alkoxy, amino, C₁-C₆ alkylamino, di C₁-C₆alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl;
Hyp is hypoxic activator; or
a tautomer or an individual isomer or a racemic or non-racemic mixture of isomers, a polymorph, a hydrate or a pharmaceutically acceptable salt or solvate thereof.

33. A compound of formula (XLIV): 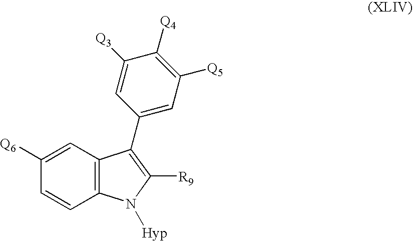
each Q₃-Q₅ is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈; or PO₃R₁₈ with the proviso that in any one compound, only one of Q₃-Q₅ is hydrogen;
R₉ is C₁-C₆ alkyl; aryl; or heteroaryl;
R₁₅ is hydrogen, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl;
R₁₈ is hydrogen, hydroxyl, C₁-C₆ alkoxy, amino, C₁-C₆ alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl; or
a tautomer or an individual isomer or a racemic or non-racemic mixture of isomers, a polymorph, a hydrate or a pharmaceutically acceptable salt or solvate thereof.

34. The compound of claim 33 wherein R₉ is:

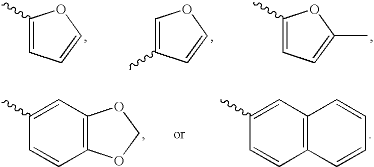

35. A compound of formula (XLV): 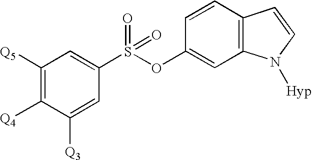
wherein each Q₃-Q₅ is hydrogen; halo; amino; C₁-C₆ alkylamino; di C₁-C₆ alkylamino; hydroxyl; C₁-C₆ alkoxy; nitro; cyano; aryl; heteroaryl; COR₁₈; SO₂R₁₈; or PO₃R₁₈ with the proviso that in any one compound, only one of Q₃-Q₅ is hydrogen;
R₁₅ is hydrogen, C₁-C₆ alkoxy, amino, C₁-C₆alkylamino, di C₁-C₆ alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl;
R₁₈ is hydrogen, hydroxyl, C₁-C₆ alkoxy, amino, C₁-C₆ alkylamino, di C₁-C₆alkylamino, NHOH, NHNH₂, C₁-C₆ alkyl, C₁-C₆ heteroalkyl, C₂-C₆ alkenyl, C₂-C₆ alkynyl, C₃-C₈ cycloalkyl, C₃-C₈ heterocyclyl, aryl, or heteroaryl;
Hyp is hypoxic activator; or
a tautomer or an individual isomer or a racemic or non-racemic mixture of isomers, a polymorph, a hydrate or a pharmaceutically acceptable salt or solvate thereof.

36. The compound of claim 35 wherein each Q₃-Q₅ is OMe.

37. A compound of formula (XLVI): 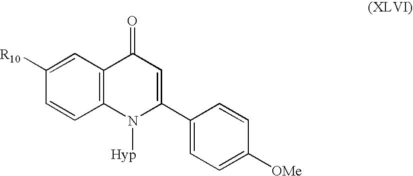
wherein R₁₀ is C₁-C₆ alkyl and Hyp is hypoxic activator and a tautomer or an individual isomer or a racemic or non-racemic mixture of isomers, a polymorph, a hydrate or a pharmaceutically acceptable salt or solvate thereof.

38. The compound of claim 37 where R₁₀ is methyl.

39. The compound of any one of claims 20-38 wherein Hyp is selected from: 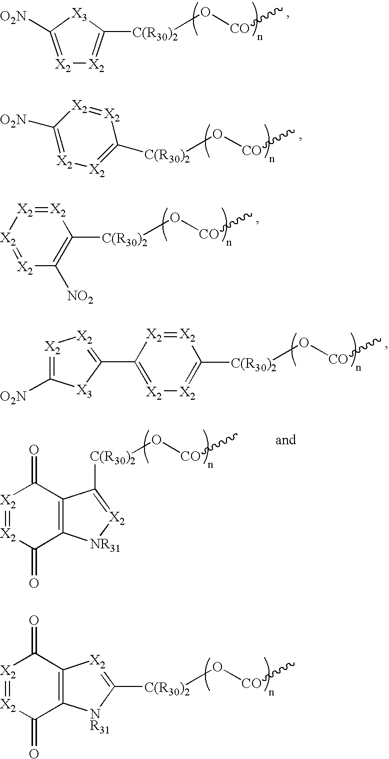
wherein each X₂ is N or CR₃₂;
X₃ is NR₃₁, S, or O;
each R₃₀ is independently hydrogen or alkyl;
R₃₁ is hydrogen, hydroxyl, C₁-C₆ alkyl or heteroalkyl, C₃-C₈ cycloalkyl, heterocyclyl, C₁-C₆ alkoxy, C₁-C₆ alkylamino, C₁-C₆ dialkylamino, aryl or heteroaryl, C₁-C₆ acyl or heteroacyl, aroyl, or heteroaroyl;
R₃₂ is hydrogen, halogen, nitro, cyano, CO₂H, C₁-C₆ alkyl or heteroalkyl, C₁-C₆ cycloalkyl, C₁-C₆ alkoxy, C₁-C₆ alkylamino, C₁-C₆ dialkylamino, aryl, CON(R₇)₂, C₁-C₆ acyl or heteroacyl, or aroyl or heteroaroyl; and
n=0, 1.

40. The compound of claim 39 wherein Hyp is selected from:

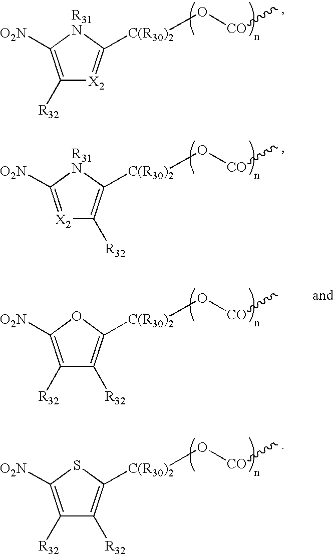

41. The compound of claim 40 wherein Hyp is 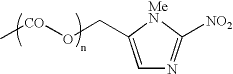
wherein n=0 or 1, provided that in -OHyp n=0.

42. A pharmaceutical composition comprising a pharmaceutically acceptable carrier and a compound according to any of claims 1-41.

43. A method of treating cancer comprising administering a therapeutically effective amount of a compound according to any of claims 1-42 alone or in combination with one or more anti-cancer agents to a subject in need of such treatment.

44. A method of treating a hyperproliferative disease comprising administering a therapeutically effective amount of a compound according to any of claims 1-42 to a subject in need of such treatment.
